(19) United States
(12) Patent Application Publication
(10) Pub. No.: US 2026/0014199 A1
(43) Pub. Date: Jan. 15, 2026

(54) MODIFIED IMMUNE CELLS

(71) Applicant: Gadeta B.V., Utrecht (NL)

(72) Inventors: Laurens Sand, Utrecht (NL); Haakan Norell, Vallentuna (SE)

(21) Appl. No.: 18/875,617

(22) PCT Filed: Jun. 19, 2023
(86) PCT No.: PCT/EP2023/066395

§ 371 Date: Dec. 16, 2024

Related U.S. Application Data
(60) Provisional application No. 63/353,434, filed on Jun. 17, 2022.

(51) Int. Cl. A61K 35/17; A61K 40/11; A61K 40/31; A61K 40/32; A61K 40/42; C07K 14/705; C07K 14/725; C07K 16/28; C07K 16/30; C12N 5/0783; C12N 15/86

(52) U.S. Cl. CPC A61K 35/17; A61K 40/11; A61K 40/31; A61K 40/32; A61K 40/4215; A61K 40/4266; C07K 14/7051; C07K 14/70517; C07K 14/70521; C07K 14/70578; C07K 16/2878; C07K 16/3007; C12N 5/0636; C12N 15/86; A61K 2239/28; C07K 2317/565; C07K 2319/03; C12N 2740/15043

(57) ABSTRACT

Modified immune cells expressing a heterodimeric gamma-delta T cell receptor (TCR) and a chimeric antigen receptor (CAR) are described. The disclosed modified immune cells enhance expression of CARs. Also disclosed are compositions and methods for making such cells, and use of such cells in treating disease.

### CROSS REFERENCE TO RELATED APPLICATION

This application claims the benefit of U.S. Provisional Application No. 63/353,434 filed on Jun. 17, 2023, the disclosure of which is hereby incorporated by reference in its entirety.

### SEQUENCE LISTING

The instant application contains a Sequence Listing which has been submitted electronically in XML format and is hereby incorporated by reference in its entirety for all purposes. The XML copy, created on Jun. 15, 2023, is referred to as 115436-764463_GDT-010-PCT.xml and is 194 kilobytes in size.

### FIELD OF THE INVENTION

The present disclosure pertains to modified immune cells, and more particularly to immune cells such as T cells modified to enhance expression of antigen receptors, methods of using such cells in treating disease, and methods and composition for making such cells.

### BACKGROUND OF THE INVENTION

Adoptive transfer of immune cells with engineered anti-tumor activity has gained traction as an effective means to fight cancer. For example, immune cells such as T cells are being modified in various ways to improve cancer targeting, with great therapeutic promise. However, numerous challenges remain including immune cell mediated toxicities, off-target effects, and tumor escape. A need exists for improved immune cell therapies with improved targeting of tumors.

### SUMMARY OF THE INVENTION

In an embodiment, the present disclosure provides a polynucleotide comprising nucleic acids encoding each of the monomers of a heterodimeric γδT-cell receptor or functional fragments thereof, and at least one nucleic acid encoding a chimeric antigen receptor (CAR), wherein said nucleic acids are operably linked to the same promoter sequence. In an embodiment, the at least one nucleic acid encoding said CAR may be inserted between the nucleic acids encoding each of said monomers. In an embodiment, the at least one nucleic acid encoding said CAR is inserted downstream, or upstream of the nucleic acids encoding both of said monomers.

In an embodiment, the promoter sequence is selected from the group consisting of EF1α, MSCV, EF1 alpha-HTLV-1 hybrid promoter, Moloney murine leukemia virus, Gibbon Ape Leukemia virus, murine mammary tumor virus, Rous sarcoma virus, MHC class II, clotting Factor IX, insulin promoter, PDX1 promoter, CD11, CD4, CD2, gp47 promoter, PGK, Beta-globin, UbC, and MND, preferably from MSCV, MMLV, EF1α, and MND.

In an embodiment, the polynucleotide further comprises at least one nucleic acid encoding at least one cis-acting regulatory element which facilitates co-expression of the CAR and the γδT-cell receptor. A cis-acting regulatory element may be selected from a 2A self-cleaving peptide, a 2A-like cis-acting hydrolase element and an IRES sequence. A 2A self-cleaving peptide may be selected from a T2A, a P2A, an E2A, and an F2A peptide.

In an embodiment, the polynucleotide comprises a first cis-acting regulatory element and a second cis-acting regulatory element. The first cis-acting regulatory element may be a T2A peptide and the second cis-acting regulatory element may be a P2A peptide. Alternatively, the first and second cis-acting regulatory elements are each independently selected from the group consisting of Picornavirus IRES, Apthovirus IRES, Hepatitis A IRES, Pestivirus IRES, Hepesvirus IRES, and combinations thereof.

In an embodiment, the polynucleotide is multicistronic. The polynucleotide may be, for example, tricistronic or tetracistronic.

In an embodiment, the CAR comprises: (i) a CAR antigen binding domain; (ii) a transmembrane domain; and (iii) an intracellular cytoplasmic binding domain; and optionally further comprises at least one of (iv) a stalk (spacer) domain; and (v) a co-stimulatory domain.

In an embodiment, the CAR antigen binding domain specifically binds to a tumor antigen, as described herein. The CAR antigen binding domain may comprise any one of an ScFV, Fab, a nanobody or any natural binding partner or fragment thereof of the tumor antigen. The tumor antigen may be selected, for example, from: BCMA, CD19, PSA, HER-2/neu, MUC1, Carcinoembryonic antigen (CEA), CA-125, Epithelial tumor antigen (ETA), Tyrosinase, Melanoma-associated antigen (MAGE). In an embodiment, the tumor antigen comprises an amino acid sequence having at least 60%, 70%, 75%, 80%, 85%, 90%, 95%, 96%, 97%, 98%, 99%, or 100% sequence identity or similarity to any one of SEQ ID NOs: 1-2 and 162. In an embodiment, the CAR antigen binding domain comprises an amino acid sequence having at least 60%, 70%, 75%, 80%, 85%, 90%, 95%, 96%, 97%, 98%, 99%, or 100% sequence identity or similarity to any one of SEQ ID NOs: 163-165.

In an embodiment, the transmembrane domain comprises a CD8 transmembrane domain, a CD137 (4-1BB transmembrane domain) or a CD28 transmembrane domain and, optionally, comprises an amino acid sequence having at least 60%, 70%, 75%, 80%, 85%, 90%, 95%, 96%, 97%, 98%, 99%, or 100% sequence identity or similarity to any one of SEQ ID NO: 5-7.

In an embodiment, the costimulatory domain of the AR comprises the intracellular domain of a costimulatory molecule selected from the group consisting of CD27, CD28, 4-1BB, OX40, CD2, ICOS, CD30, and any combination thereof. In an embodiment, the intracellular domain of a costimulatory molecule comprises an amino acid sequence having at least 60%, 70%, 75%, 80%, 85%, 90%, 95%, 96%, 97%, 98%, 99%, or 100% sequence identity or similarity to any one of SEQ ID NOS: 10 or 11.

In an embodiment, the intracellular cytoplasmic binding domain of the CAR comprises a domain selected from a group consisting of CD3e, CD3z, FCE1RG, and any domain comprising an ITAM or hemiTAM. In an embodiment, the intracellular cytoplasmic binding domain comprises an amino acid sequence having at least 60%, 70%, 75%, 80%, 85%, 90%, 95%, 96%, 97%, 98%, 99%, or 100% sequence identity or similarity to SEQ ID NO: 8 or 9.

In an embodiment, the stalk domain of the CAR comprises a CD8 stalk or a CD28 stalk. In an embodiment, the stalk domain comprises a sequence which has at least 60%, 70%, 75%, 80%, 85%, 90%, 95%, 96%, 97%, 98%, 99%, or 100% sequence identity or similarity to SEQ ID NO: 3 or 4.

In an embodiment, the CAR comprises an amino acid sequence having at least 60%, 70%, 75%, 80%, 85%, 90%, 95%, 96%, 97%, 98%, 99%, or 100% sequence identity or similarity to any one of SEQ ID NOs: 29, 30 and 31.

In an embodiment, the heterodimeric γδT-cell receptor comprises: a) a γ-chain selected from the group consisting of γ2, γ3, γ4, γ5, γ8, γ9, and γ11, and derivatives, variations or fragments thereof; (b) a δ-chain selected from the group consisting of δ1, δ2, δ3, and δ5, and derivatives, variations or fragments thereof; or (c) any combination of (a) and (b). In an embodiment, the heterodimeric γδT-cell receptor comprises a γ982 or a γ405 receptor.

In an embodiment, the heterodimeric γδT-cell receptor comprises a γ-chain having a complementary determining region 3 (CDR3) having at least 60%, 70%, 75%, 80%, 85%, 90%, 95%, 96%, 97%, 98%, 99%, or 100% sequence identity or similarity with an amino acid sequence of SEQ ID NO: 37 and/or a δ-chain having a complementary determining region 3 (CDR3) having at least 60%, 70%, 75%, 80%, 85%, 90%, 95%, 96%, 97%, 98%, 99%, or 100% sequence identity or similarity with an amino acid sequence of SEQ ID NO: 34. In an embodiment, (a) the γ-chain of the heterodimeric γδT-cell receptor further comprises a complementary determining region 1 (CDR1) having at least 60%, 70%, 80%, 90%, 95%, or 100% identity or similarity with an amino acid sequence of SEQ ID NO: 166, a complementary determining region 2 (CDR2) having at least 60%, 70%, 75%, 80%, 85%, 90%, 95%, 96%, 97%, 98%, 99%, or 100% sequence identity or similarity with an amino acid sequence of SEQ ID NO: 167, or a combination thereof; and/or (b) the δ-chain further comprises a complementary determining region 1 (CDR1) having at least 60%, 70%, 80%, 90%, 95%, or 100% identity or similarity with an amino acid sequence of SEQ ID NO: 168, a complementary determining region 2 (CDR2) having at least 60%, 70%, 75%, 80%, 85%, 90%, 95%, 96%, 97%, 98%, 99%, or 100% sequence identity or similarity with an amino acid sequence of EKD, or a combination thereof. In an embodiment, the γ-chain of the heterodimeric γδT-cell receptor comprises an amino acid sequence having at least 60%, 70%, 80%, 90%, 95%, or 100% identity or similarity with SEQ ID NO: 41; and/or the δ-chain of the heterodimeric γδT-cell receptor comprise an amino acid sequence having at least 60%, 70%, 75%, 80%, 85%, 90%, 95%, 96%, 97%, 98%, 99%, or 100% sequence identity or similarity with SEQ ID NO: 40.

In an embodiment, the heterodimeric γδT-cell receptor comprises a γ-chain having a complementary determining region 3 (CDR3) having at least 60%, 70%, 80%, 90%, 95%, or 100% identity or similarity with an amino acid sequence of SEQ ID NO: 51; and/or a δ-chain having a complementary determining region 3 (CDR3) having at least 60%, 70%, 75%, 80%, 85%, 90%, 95%, 96%, 97%, 98%, 99%, or 100% sequence identity or similarity with an amino acid sequence of SEQ ID NO: 52. In an embodiment, a) the γ-chain of the heterodimeric γδT-cell receptor further comprises a complementary determining region 1 (CDR1) having at least 60%, 70%, 80%, 90%, 95%, or 100% identity or similarity with an amino acid sequence of SEQ ID NO: 169, a complementary determining region 2 (CDR2) having at least 60%, 70%, 75%, 80%, 85%, 90%, 95%, 96%, 97%, 98%, 99%, or 100% sequence identity or similarity with an amino acid sequence of SEQ ID NO: 170, or a combination thereof; and/or (b) the δ-chain further comprises a complementary determining region 1 (CDR1) having at least 60%, 70%, 80%, 90%, 95%, or 100% identity or similarity with an amino acid sequence of SEQ ID NO: 171, a complementary determining region 2 (CDR2) having at least 60%, 70%, 75%, 80%, 85%, 90%, 95%, 96%, 97%, 98%, 99%, or 100% sequence identity or similarity with an amino acid sequence of SEQ ID NO: 172, or a combination thereof. In an embodiment, the γ-chain of the heterodimeric γδT-cell receptor comprises an amino acid sequence having at 60%, 70%, 75%, 80%, 85%, 90%, 95%, 96%, 97%, 98%, 99%, or 100% sequence identity or similarity with SEQ ID NO: 62; and/or the δ-chain of the heterodimeric γδT-cell receptor comprise an amino acid sequence having at least 60%, 70%, 75%, 80%, 85%, 90%, 95%, 96%, 97%, 98%, 99%, or 100% sequence identity or similarity with SEQ ID NO: 61.

In an embodiment, γδT-cell receptor binds to a first antigen, and the CAR binds to a second antigen. In an embodiment, the γδT-cell receptor binds to cluster of differentiation 277 (CD277), an endothelial protein c receptor (EPCR), an annexin A2 protein or any combination thereof; and the CAR binds to B cell maturation antigen (BCMA), cluster of differentiation 19 (CD19), carcinoembryonic antigen (CEA) or any combination thereof.

In an embodiment, one or both of the monomers of the heterodimeric γδT-cell receptor have a deletion of all or a portion of the variable domain.

In an embodiment, the polynucleotide of the present disclosure encodes a polypeptide having at least 60%, 70%, 75%, 80%, 85%, 90%, 95%, 96%, 97%, 98%, 99%, or 100% sequence identity or similarity with any one of SEQ ID NOs: 112, 152, 158, and 174.

In an embodiment, the present disclosure provides a vector comprising a polynucleotide as described herein.

In an embodiment, the vector comprises a plasmid, a retroviral vector, lentiviral vector, an adenoviral vector, or an adenovirus associated viral (AAV) vector.

In an embodiment, the vector comprises a nucleic acid sequence having at least 60%, 70%, 75%, 80%, 85%, 90%, 95%, 96%, 97%, 98%, 99%, or 100% sequence identity or similarity with any one of SEQ ID NO: 111, 151, 157 and 173.

In an embodiment, the present disclosure also provides an immune cell comprising any polynucleotide as described herein or a vector as described herein. In an embodiment the immune cell co-expresses a γδT-cell receptor and a CAR as described herein. The immune cell may be, in non-limiting example, a T cell. The immune cell may be in a preferred, but non-limiting example, an ap T-cell.

In an embodiment, in an immune cell as described herein, the level of CAR expression in the cell is increased relative to the level of CAR expression in a cell lacking co-expression of the γδT-cell receptor and the CAR.

In an embodiment, the present disclosure also provides a population of cells comprising a plurality of any immune cells as described herein.

In an embodiment of an immune cell or population of cells, with respect to a tumor cell having an antigen that binds to said CAR of said cells, said cells exhibit any one or more of increased cytotoxicity, antitumor activity, and/or tumor cell killing, and/or proliferation, cellular survival, or persistence, as compared to cells that do not co-express said γδT-cell receptor and said CAR.

In an embodiment, the present disclosure also provides a method of increasing expression and/or function of a CAR in an immune cell, the method comprising: introducing to the immune cell any polynucleotide as described herein, or a vector as described herein. The level of CAR expression in the cell is increased relative to the level of CAR expression in an immune cell lacking co-expression of the γδT-cell receptor and the CAR. In the method, the immune cell may be a T cell such as, in a non-limiting example, an αβ T cell.

In an embodiment, increasing function of the CAR in the immune cell comprises increasing any one or more of cytotoxicity, antitumor activity, and/or tumor cell killing with respect to a cancer cell having an antigen that binds to the CAR of the cells, and/or increasing proliferation, cellular survival, or persistence of the immune cell, as compared to an immune cell that does not co-express the γδT-cell receptor with the CAR.

In an embodiment, after introducing a polynucleotide as described herein, or a vector as described herein to the immune cell, the method may further comprise maintaining the immune cell for a time and under conditions sufficient for the immune cell to co-express the γδT-cell receptor and the CAR.

In an embodiment, increasing expression of the CAR in the immune cell comprises increasing the level of CAR expression in the immune cell relative to the level of CAR expression in an immune cell lacking co-expression of the γδT-cell receptor.

In an embodiment, the present disclosure provides a method for treating a cancer in a subject in need thereof, the method comprising: administering to the subject an effective amount of an immune cell as described herein, or a population of immune cells as described herein, thereby inducing anti-cancer immunity in the subject.

In an embodiment, the present disclosure provides a method for stimulating an immune cell-mediated immune response to a cell population in a subject in need thereof, the method comprising: administering to the subject an effective amount of an immune cell as described herein, or a population of immune cells as described herein, thereby stimulating in the subject an immune cell-mediated immune response to the cell population.

In an embodiment, the subject is a mammal, and in non-limiting example, the subject may be a human.

In an embodiment, an immune cell as described herein, or a population of immune cells as described herein, is used in the treatment of a cancer.

In an embodiment, one or more of the immune cells as described herein, or a population of immune cells as described herein, is used in stimulating an immune cell-mediated immune response to a cell population or tissue.

In an embodiment, the present disclosure provides a polynucleotide comprising A or B, wherein:
a. (A) is a nucleic acid represented by (i)-(ii)-(iii), wherein:
   (i) is a nucleic acid encoding a γ chain of a γδT-cell receptor or a functional fragment thereof,
   (ii) is at least one nucleic acid encoding a CAR or a functional fragment thereof, and;
   (iii) is a nucleic acid encoding a δ chain of a γδT-cell receptor or a functional fragment thereof, wherein (ii) is positioned between (i) and (iii), is positioned downstream of both (i) and (iii), or is positioned upstream of both (i) and (iii); and
b. (B) is a nucleic acid represented by (iv)-(v)-(vi), wherein:
   (iv) is a nucleic acid encoding a δ chain of a γδT-cell receptor or a functional fragment thereof,
   (v) is at least one nucleic acid encoding a CAR or a functional fragment thereof, and;
   (vi) is a nucleic acid encoding a γ chain of a γδT-cell receptor or a functional fragment thereof, wherein (v) is positioned between (iv) and (vi), is positioned downstream of both (iv) and (vi), or is positioned upstream of both (iv) and (vi).

In an embodiment, in a polynucleotide comprising A or B, (i) and (vi) are nucleic acids comprising a nucleotide sequence encoding a polypeptide having at least 60%, 70%, 80%, 90%, 95%, or 100% identity or similarity with an amino acid sequence selected from SEQ ID NOS: 35, 36, 37, 39, 41, 43, 44, 45, 46, 51, 54, 56, 58, 60, 62, 63, 65, 67, 69, 71, 73, 75, 77, 79, 80, 82, 86, 87, 90, 91, 94, 95, 98, 99, 101, 102, 125, 126, 129, 130, 133, 134, 137, 138, 141, 142, 145, 146, 149, and 150 preferably selected from SEQ ID NOs: 35, 36, 37, 44, 45, 46, 51, 63, 65, 67, 69, 77, 80, 86, 87, 90, 91, 94, 95, 98, 99, 101, 102, 125, 126, 129, 130, 133, 134, 137, 138, 141, 142, 145, 146, 149, and 150 and/or; and (iii) and (iv) are nucleic acids comprising a nucleotide sequence encoding a polypeptide having at least 60%, 70%, 80%, 90%, 95%, or 100% identity or similarity with an amino acid sequence selected from SEQ ID NOs: 32, 33, 34, 38, 40, 42, 47, 48, 49, 50, 52, 53, 55, 57, 59, 61, 64, 66, 68, 70, 72, 74, 76, 78, 81, 83, 84, 85, 88, 89, 92, 93, 96, 97, 100, 123, 124, 127, 128, 131, 132, 135, 136, 139, 140, 143, 144, 147, and 148 preferably selected from SEQ ID NOs: 32, 33, 34, 47, 48, 49, 50, 52, 64, 66, 68, 76, 81, 84, 85, 88, 89, 92, 93, 96, 97, 100, 123, 124, 127, 128, 131, 132, 135, 136, 139, 140, 143, 144, 147, and 148.

In an embodiment, in a polynucleotide comprising A or B, (i) and (vi) are nucleic acids comprising a nucleotide sequence encoding a polypeptide having at least 60%, 70%, 80%, 90%, 95%, or 100% identity or similarity with an amino acid sequence of SEQ ID NOs: 37 and, optionally, further comprising a nucleotide sequence encoding a polypeptide having at least 60%, 70%, 75%, 80%, 85%, 90%, 95%, 96%, 97%, 98%, 99%, or 100% sequence identity or similarity to SEQ ID NO: 166 and/or a nucleotide sequence encoding a polypeptide having at least 60%, 70%, 75%, 80%, 85%, 90%, 95%, 96%, 97%, 98%, 99%, or 100% sequence identity or similarity to SEQ ID NO: 167, and/or (iii) and (iv) are nucleic acids comprising a nucleotide sequence encoding a polypeptide having at least 60%, 70%, 75%, 80%, 85%, 90%, 95%, 96%, 97%, 98%, 99%, or 100% sequence identity or similarity with an amino acid sequence of SEQ ID NO: 34 and, optionally, further comprising a nucleotide sequence encoding a polypeptide having at least 60%, 70%, 75%, 80%, 85%, 90%, 95%, 96%, 97%, 98%, 99%, or 100% sequence identity or similarity to SEQ ID NO: 168 and/or a nucleotide sequence encoding a polypeptide having at least 60%, 70%, 75%, 80%, 85%, 90%, 95%, 96%, 97%, 98%, 99%, or 100% sequence identity or similarity to an amino acid sequence of EKD.

In an embodiment, in a polynucleotide comprising A or B, (i) and (vi) are nucleic acids comprising a nucleotide sequence encoding a polypeptide having at least 60%, 70%, 75%, 80%, 85%, 90%, 95%, 96%, 97%, 98%, 99%, or 100% sequence identity or similarity with an amino acid sequence of SEQ ID NOs: 41 and/or iii) and (iv) are nucleic acids comprising a nucleotide sequence encoding a polypeptide having at least 60%, 70%, 75%, 80%, 85%, 90%, 95%, 96%, 97%, 98%, 99%, or 100% sequence identity or similarity with an amino acid sequence of SEQ ID NO: 40.

In an embodiment, in a polynucleotide comprising A or B, (i) and (vi) are nucleic acids comprising a nucleotide sequence encoding a polypeptide having at least 60%, 70%, 80%, 90%, 95%, or 100% identity or similarity with an amino acid sequence of SEQ ID NOs: 51 and, optionally, further comprising a nucleotide sequence encoding a polypeptide having at least 60%, 70%, 75%, 80%, 85%, 90%, 95%, 96%, 97%, 98%, 99%, or 100% sequence identity or similarity to SEQ ID NO: 169 and/or a nucleotide sequence encoding a polypeptide having at least 60%, 70%, 75%, 80%, 85%, 90%, 95%, 96%, 97%, 98%, 99%, or 100% sequence identity or similarity to SEQ ID NO: 170, and/or (iii) and (iv) are nucleic acids comprising a nucleotide sequence encoding a polypeptide having at least 60%, 70%, 80%, 90%, 95%, or 100% identity or similarity with an amino acid sequence of SEQ ID NO: 52 and, optionally, further comprising a nucleotide sequence encoding a polypeptide having at least 60%, 70%, 75%, 80%, 85%, 90%, 95%, 96%, 97%, 98%, 99%, or 100% sequence identity or similarity to SEQ ID NO: 171 and/or a nucleotide sequence encoding a polypeptide having at least 60%, 70%, 75%, 80%, 85%, 90%, 95%, 96%, 97%, 98%, 99%, or 100% sequence identity or similarity to SEQ ID NO: 172.

In an embodiment, in a polynucleotide comprising A or B, (i) and (vi) are nucleic acids comprising a nucleotide sequence encoding a polypeptide having at least 60%, 70%, 75%, 80%, 85%, 90%, 95%, 96%, 97%, 98%, 99%, or 100% sequence identity or similarity with an amino acid sequence of SEQ ID NOs: 62 and/or iii) and (iv) are nucleic acids comprising a nucleotide sequence encoding a polypeptide having at least 60%, 70%, 75%, 80%, 85%, 90%, 95%, 96%, 97%, 98%, 99%, or 100% sequence identity or similarity with an amino acid sequence of SEQ ID NO: 61.

In an embodiment, a polynucleotide comprising A or B (ii) or (v) are nucleic acids comprising a nucleotide sequence encoding a polypeptide having at least 60%, 70%, 75%, 80%, 85%, 90%, 95%, 96%, 97%, 98%, 99%, or 100% sequence identity or similarity to any one of SEQ ID NOs: 3, 4, 5, 6, 7, 8, 9, 10, 11, 29, 30, 31, 154, 156, 160, 163, 164, and 165. In an embodiment, (ii) or (v) are nucleic acids comprising a nucleotide sequence encoding a polypeptide having at least 60%, 70%, 75%, 80%, 85%, 90%, 95%, 96%, 97%, 98%, 99%, or 100% sequence identity or similarity to any one of SEQ ID NOs: 29, 30 or 31.

In an embodiment, a polynucleotide of A or B may encode for a polypeptide having a polypeptide having at least 60%, 70%, 75%, 80%, 85%, 90%, 95%, 96%, 97%, 98%, 99%, or 100% sequence identity or similarity with any one of SEQ ID NOs: 112, 152, 158, and 174.

The present disclosure provides a vector comprising a polynucleotide comprising A or B. The vector may comprise a plasmid, a retroviral vector, lentiviral vector, an adenoviral vector, or an adenovirus associated viral (AAV) vector. In an embodiment, the vector comprises a nucleic acid sequence having at least 60%, 70%, 75%, 80%, 85%, 90%, 95%, 96%, 97%, 98%, 99%, or 100% sequence identity or similarity with any one of SEQ ID NO: 111, 151 157, and 173.

In an embodiment, an immune cell comprises a polynucleotide comprising A or B, or comprises a vector comprising a polynucleotide comprising A or B. The immune cell may be a T cell. In an embodiment, the immune cell may be an ap T-cell.

In an embodiment, the present disclosure provides a method of increasing expression and/or function of a CAR in an immune cell, the method comprising: introducing to the immune cell the polynucleotide comprising A or B, or introducing the vector comprising the polynucleotide comprising A or B. In non-limiting example, the immune cell may be a T cell. In an embodiment, the immune cell may be an αβ T-cell.

In an embodiment, the present disclosure provides one or more immune cells as described herein, for use in the treatment of a cancer.

In an embodiment, the present disclosure provides one or more immune cells as described herein, for use in stimulating an immune cell-mediated immune response to a cell population or tissue.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE FIGURES

A complete understanding of the features and advantages of the present disclosure will be obtained by reference to the following detailed description, and the accompanying drawings of which:

FIG. 1 is a schematic illustration of some exemplary polycistronic constructs used to express monomers of a heterodimeric γδT-cell receptor or functional fragments thereof, and at least one chimeric antigen receptor (CAR).

FIG. 2 is a schematic map of pLenti6.3_v3.

FIG. 3 provides flow cytometry data showing enhanced expression on T cell of γ-TCR-BCMA-BBz-CAR-δ-TCR, as compared to expression of BCMA-BBz-CAR alone.

FIG. 4 shows the cytotoxic effects of T cell expressing BCMA-BBz-CAR_eGFP or T-cells co-expressing BCMA-BBz-CAR with monomers of γδ-TCR. The T-cells were co-incubated with target Daudi tumor cells (not expressing BCMA) with addition of 10 UM pamidronate (γδ-TCR target) and without (not recognized by γδ-TCR) at a ratio 1:1. The left panel shows the data with addition of pamidronate (10 μm, PAM). (N=3, *P<0.01)

FIG. 5 shows the effect of co-expressing BCMA-BBz CAR with different γδ-TCR subunits on cytotoxicity of T cells in a single vector or double vector transduction. T cells expressing BCMA-BBz CAR alone or in combination with γδ-TCR on the same vector on a separate vector were co-incubated with BCMA-CAR target MM. 1S tumor cells at 1:1 E/T ratio. Every 3 days T cells were transferred to fresh target cells. Serial stimulation of T-cells was continued for 8 stimulations. Bars represent mean±SD of triplicates in a single experiment.

FIGS. 6A-6C show the effect of co-expressing BCMA-BBz CAR with different expression having a T2A- or P2A-intracellular scar after ribosomal skipping with or without co-expression of different γδ-TCR subunits in T cells. T cells were transduced with lentivirus encoding the indicated constructs and the mean fluorescent intensity (MFI) of BCMA-CAR expression was analyzed and compared for the different constructs. Data from three donors is shown in bars±SD from a single experiment (FIG. 6A). Paired MFI of BCMA-BBz CAR in having a P2A (FIG. 6B) or T2A (FIG. 6C) scar in the presence or absence of different γδ-TCR subunits per donor is shown. P-value is provided, and significant threshold was set at p=0.05. (ns=not significant).

FIGS. 7A-7B show lysis of RPMI-8226 LucTOM target cells by T cells expressing the BCMA-BBz CAR with or without a T2A, or P2A scar in combination with or without different γδ-TCR subunits. T cells expressing different γδ-TCR subunits alone and untransduced T cells were added as controls. RPMI-8226 LucTOM tumor cells, which do not express CD277, were co-cultured with indicated T cells with and effector to target cell ratio of 1:1. Every 3 to 4 days T cells were transferred to fresh target cells. Serial stimulation of T-cells was continued for 8 stimulations. Data is plotted as the mean relative luciferase light unit value for each stimulation±SD of triplicates in a single experiment (FIG. 7A) and normalized to percentage of lysis (FIG. 7B).

FIGS. 8A-8B shows lysis of RPMI-8226 LucTOM target cells, lacking expression of CD277, by T cells expressing the BCMA-BBz CAR with or without a T2A, or P2A scar in combination with or without different γδ-TCR subunits. T cells expressing different γδ-TCR subunits alone and untransduced T cells were added as controls. RPMI-8226 LucTOM tumor cells, which do not express CD277, were co-cultured with indicated T cells with an effector to target cell ratio of 1:1. Every 3 to 4 days T cells were transferred to fresh target cells. Serial stimulation of T-cells was continued for 8 stimulations. Data is plotted as the mean relative luciferase light unit value for each stimulation±SD of triplicates in a single experiment (FIG. 8A) and normalized to percentage of lysis (FIG. 8B).

FIGS. 9A-9B show lysis of HT-29 LucTOM target cells by T cells expressing the CEA-CAR having the P2A scar in combination with or without different γδ-TCR subunits. T cells expressing different γδ-TCR subunits alone and untransduced T cells were added as controls. HT-29 LucTOM tumor cells, were co-cultured with indicated T cells with an effector to target cell ratio of 1:1-1:8. Every 3 to 4 days T cells were transferred to fresh target cells. Serial stimulation of T-cells was continued for 4 stimulations. The mean percentage of lysis of target cells±SD of triplicates, in a single experiment when an E:T of 1:1 was used was plotted (FIG. 9A). The amount of IFN release produced by activated T cells was determined by ELISA after stimulation round 1 when a 1:1-1:8 E/T ratio was used (FIG. 9B).

### DETAILED DESCRIPTION

Engineered cells hold great potential both for research and therapeutic applications. For example, certain engineered immune cells have provided landmark advances in the treatment of some types of cancer for which no effective treatments were previously available. However, despite increased efforts to generate new and more advanced engineered cells, a number of challenges remain that limit success in the field. Examples of these challenges include difficulties in generating sufficient numbers of the desired engineered cells, limited proliferative ability or lifespan of the engineered cells, limited fitness of the engineered cells, limited induction of effector function upon antigen recognition, and exhaustion.

Accordingly, disclosed herein are engineered cells, compositions and methods based in part on the surprising discovery that a polycistronic construct used to induce co-expression of a chimeric antigen receptor (CAR) together with γδ-TCR monomers, greatly enhances per-cell expression of the CAR(s) and improves the functional efficacy of immune cells, such as T-cells expressing the CAR, particularly when the CAR is positioned between the γδ-TCR monomers. Improved surface expression of a heterodimeric receptor may be assessed by any standard technique available to the skilled person discussed earlier herein, such as flow cytometry, FACS, and the like. Further non-limiting examples are provided in the experimental section. Among the various aspects disclosed herein are polynucleotides which encode a heterodimeric receptor as described further below, and a CAR.

Encompassed by the disclosure are polynucleotides comprising nucleic acids encoding each of the monomers of a heterodimeric γδ T cell receptor, or functional fragments thereof, and at least one nucleic acid encoding a chimeric antigen receptor (CAR), the nucleic acid operably linked to the same promoter. The disclosure encompasses vectors comprising such polynucleotides, cells comprising such polynucleotides or such vectors, methods for making such cells, compositions comprising the cells, and methods using the cells to treat diseases such as cancer. The current disclosure also encompasses methods of enhancing expression and/or functionality of a CAR and use of such enhanced cells in the treatment of disease.

### I. Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements. Wherever the terms “comprising” or “including” are used, it should be understood the disclosure also expressly contemplates and encompasses additional embodiments “consisting of” the disclosed elements, in which additional elements other than the listed elements are not included.

The terms “nucleic acid”, “nucleic acid molecule”, and “polynucleotide” are used interchangeably herein. The terms “nucleic acid encoding . . . ”, or “nucleic acid molecule encoding . . . ” should be understood as referring to the sequence of nucleotides which encodes a polypeptide.

A polynucleotide described herein may comprise one or more nucleic acids each encoding a polypeptide, all operably linked to (i.e., in a functional relationship with) one or more regulatory sequences, such as a promoter. Such a polynucleotide may alternatively be referred to herein as a “nucleic acid construct” or “construct”.

As used herein, a regulatory sequence refers to any genetic element that is known to the skilled person to drive or otherwise regulate expression of nucleic acids in a cell. Such sequences include without limitation promoters, transcription terminators, enhancers, repressors, silencers, kozak sequences, polyA sequences, and the like. A regulatory sequence can, for example, be inducible, non-inducible, constitutive, cell-cycle regulated, metabolically regulated, and the like. A regulatory sequence may be a promoter. Non-limiting examples of suitable promoters include EF1α, MSCV, EF1 alpha-HTLV-1 hybrid promoter, Moloney murine leukemia virus (MoMuLV or MMLV), Gibbon Ape Leukemia virus (GALV), murine mammary tumor virus (MuMTV or MMTV), Rous sarcoma virus (RSV), MHC class II, clotting Factor IX, insulin promoter, PDX1 promoter, CD11, CD4, CD2, gp47 promoter, PGK, Beta-globin, UbC, MND, and derivatives (i.e. variants) thereof. Examples of these promoters are further described in Poletti and Mavilio (2021), Viruses 13:8; 1526, Kuroda et al. (2008), J Gene Med 10 (11): 1163-1175, Milone et al. (2009), Mol Ther 17:8; 1453-1464, and Klein et al. (2008), J Biomed Biotechnol 683505, all of which are incorporated herein by reference in their entireties.

A polynucleotide described herein may be multicistronic. “Multicistronic” (alternatively referred to herein as “polycistronic”) can refer to the transcription of the polynucleotide resulting in an mRNA from which at least two distinct polypeptides are translated. This, for example, may be achieved by a polynucleotide comprising at least two nucleic acids encoding distinct polypeptides, preferably operably linked to the same promoter. In some embodiments, at least two, at least three, at least four, at least five, or at least six, preferably at least three or at least four, polypeptides are expressed by a polynucleotide described herein. A polynucleotide described herein may be tricistronic (i.e., three distinct polypeptides may be expressed). A polynucleotide described herein may be tetracistronic (i.e., four distinct polypeptides may be expressed). A multicistronic polynucleotide may comprise additional nucleotide sequences facilitating the co-expression of the encoded polypeptides, such as cis-acting regulatory elements described later herein. A polynucleotide may be incorporated in a vector as described later herein.

A “wild type” protein amino acid sequence can refer to a sequence that is naturally occurring and encoded by a germline genome. A species can have one wild type sequence, or two or more wild type sequences (for example, with one canonical wild type sequence and one or more non-canonical wild type sequences). A wild type protein amino acid sequence can be a mature form of a protein that has been processed to remove N-terminal and/or C-terminal residues, for example, to remove a signal peptide.

An amino acid sequence that is “derived from” a wild type sequence or other amino acid sequence disclosed herein can refer to an amino acid sequence that differs by one or more amino acids compared to the reference amino acid sequence, for example, containing one or more amino acid insertions, deletions, or substitutions as disclosed herein. The terms “derivative,” “variant,” “variations” and “fragment,” when used herein with reference to a polypeptide, refers to a polypeptide related to a wild type polypeptide, for example either by amino acid sequence, structure (e.g., secondary and/or tertiary), activity (e.g., enzymatic activity) and/or function. Derivatives, variants, variations and fragments of a polypeptide can comprise one or more amino acid variations (e.g., mutations, insertions, and deletions), truncations, modifications, or combinations thereof compared to a wild type polypeptide. A part or fragment of a polypeptide may correspond to at least 1%, at least 2%, at least 3%, at least 4%, at least 5%, at least 10%, at least 20%, at least 30%, at least 40% of the length of a polypeptide, such as a polypeptide having an amino acid sequence identified by a specific SEQ ID NO., or having at least 50%, or at least 60%, or at least 70%, or at least 80%, or at least 90% of the length (in amino acids) of the polypeptide.

Within the context of the present application, a protein is represented by an amino acid sequence, and correspondingly a nucleic acid molecule or a polynucleotide is represented by a nucleic acid sequence. Identity and similarity between sequences: throughout this application, it should be understood that for each reference to a specific amino acid sequence using a unique sequence identifier (SEQ ID NO.), the sequence may be replaced by: a polypeptide represented by an amino acid sequence comprising a sequence that has at least 60% sequence identity or similarity with 5 fold, at least 10 fold, at least 20 fold, at least 50 fold, at least 100 fold, or at least 1000 fold he reference amino acid sequence. Another preferred level of sequence identity or similarity is 65%. Another preferred level of sequence identity or similarity is 70%. Another preferred level of sequence identity or similarity is 75%. Another preferred level of sequence identity or similarity is 80%. Another preferred level of sequence identity or similarity is 85%. Another preferred level of sequence identity or similarity is 90%. Another preferred level of sequence identity or similarity is 95%. Another preferred level of sequence identity or similarity is 98%. Another preferred level of sequence identity or similarity is 99%.

Each amino acid sequence described herein by virtue of its identity or similarity percentage with a given amino acid sequence respectively has in a further preferred embodiment an identity or a similarity of at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% with the given nucleotide or amino acid sequence, respectively. The terms “homology”, “sequence identity” and the like are used interchangeably herein. Sequence identity is described herein as a relationship between two or more amino acid (polypeptide or protein) sequences, or two or more nucleic acid (polynucleotide) sequences, as determined by comparing the sequences. In a preferred embodiment, sequence identity is calculated based on the full length (in amino acids or nucleotides) of two given SEQ ID NOs or based on a portion thereof, more preferably based on the full length. A portion of a full length sequence may be referred to as fragment, and preferably means at least 50%, 60%, 70%, 80%, 90%, or 100% of the length (in amino acids or nucleotides) of a reference sequence. “Identity” also refers to the degree of sequence relatedness between two amino acid or between two nucleic acid sequences, as the case may be, as determined by the match between strings of such sequences. The degree of sequence identity between two sequences can be determined, for example, by comparing the two sequences using computer programs commonly employed for this purpose, such as global or local alignment algorithms. Non-limiting examples include BLASTp, BLASTn, Clustal W, MAFFT, Clustal Omega, AlignMe, Praline, GAP, BESTFIT, or another suitable method or algorithm. A Needleman and Wunsch global alignment algorithm can be used to align two sequences over their entire length or part thereof (part thereof may mean at least 50%, 60%, 70%, 80%, 90% of the length of the sequence), maximizing the number of matches and minimizes the number of gaps. Default settings can be used and preferred program is Needle for pairwise alignment (in an embodiment, EMBOSS Needle 6.6.0.0, gap open penalty 10, gap extent penalty: 0.5, end gap penalty: false, end gap open penalty: 10, end gap extent penalty: 0.5 is used) and MAFFT for multiple sequence alignment (in an embodiment, MAFFT v7Default value is: BLOSUM62 [bl62], Gap Open: 1.53, Gap extension: 0.123, Order: aligned, Tree rebuilding number: 2, Guide tree output: ON [true], Max iterate: 2, Perform FFTS: none is used).

“Similarity” between two amino acid sequences is determined by comparing the amino acid sequence and its conserved amino acid substitutes of one polypeptide to the sequence of a second polypeptide. Similar algorithms used for determination of sequence identity may be used for determination of sequence similarity. Optionally, in determining the degree of amino acid similarity, the skilled person may also take into account so-called conservative amino acid substitutions. As used herein, “conservative” amino acid substitutions refer to the interchangeability of residues having similar side chains. Examples of classes of amino acid residues for conservative substitutions are given in the Tables below:

| | |
|---|---|
| Acidic Residues | Asp (D) and Glu (E) |
| Basic Residues | Lys (K), Arg (R), and His (H) |
| Hydrophilic Uncharged Residues | Ser (S), Thr (T), Asn (N), and |
| | Gln (Q) |
| Aliphatic Uncharged Residues | Gly (G), Ala (A), Val (V), Leu (L), |
| | and Ile (I) |
| Non-polar Uncharged Residues | Cys (C), Met (M), and Pro (P) |
| Aromatic Residues | Phe (F), Tyr (Y), and Trp (W) |

Alternative conservative amino acid residue substitution classes:

| | | | |
|---|---|---|---|
| 1 | A | S | T |
| 2 | D | E | |
| 3 | N | Q | |
| 4 | R | K | |
| 5 | I | L | M |
| 6 | F | Y | W |

Alternative physical and functional classifications of amino acid residues:

| | |
|---|---|
| Alcohol group-containing residues | S and T |
| Aliphatic residues | I, L, V, and M |
| Cycloalkenyl-associated residues | F, H, W, and Y |
| Hydrophobic residues | A, C, F, G, H, I, L, M, R, T, V, |
| | W, and Y |
| Negatively charged residues | D and E |
| Polar residues | C, D, E, H, K, N, Q, R, S, and T |
| Positively charged residues | H, K, and R |
| Small residues | A, C, D, G, N, P, S, T, and V |
| Very small residues | A, G, and S |
| Residues involved in turn formation | A, C, D, E, G, H, K, N, Q, R, S, |
| | P and T |
| Flexible residues | Q, T, K, S, G, P, D, E, and R |

For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to Ser; Arg to Lys; Asn to Gln or His; Asp to Glu; Cys to Ser or Ala; Gln to Asn; Glu to Asp; Gly to Pro; His to Asn or Gln; Ile to Leu or Val; Leu to Ile or Val; Lys to Arg; Gln or Glu; Met to Leu or Ile; Phe to Met, Leu or Tyr; Ser to Thr; Thr to Ser; Trp to Tyr; Tyr to Trp or Phe; and, Val to Ile or Leu.

As used herein, the term “chimeric antigen receptor” or “CAR” refers to an artificial exogenous antigen recognition receptor that can induce signaling in an engineered cell that expresses the CAR upon binding of the CAR to an antigen, for example, an antigen associated with a cancer or infectious disease. A CAR generally induces signaling in the engineered cell that expresses the CAR but not in a cell that expresses or presents the antigen bound by the CAR. A CAR comprises at least one extracellular targeting domain, at least one transmembrane domain, and at least one intracellular signaling domain. In some cases, a CAR comprises a hinge domain. A CAR extracellular targeting domain can be, comprise, or be derived from, for example, a monoclonal antibody, a recombinant antibody, a human antibody, a humanized antibody, or a functional derivative, variant or fragment thereof, including, but not limited to, a heavy chain variable domain (VH), a light chain variable domain (VL), a Fab, a Fab′, a F(ab′)2, an Fv, a single-chain FV (scFv), a minibody, a diabody, a single-domain antibody such as a VHH, and any combination thereof. A CAR extracellular targeting domain can be, comprise, or be derived from, for example, a DARPin, a non-antibody domain (e.g., from or derived from a receptor or a receptor ligand, for example, APRIL). The intracellular signaling domain of a CAR can induce or reduce activity of an engineered cell comprising the CAR. An intracellular signaling domain of a CAR can be or can comprise a truncated portion of a signaling domain of another molecule. In some cases, intracellular domain of the CAR can be involved in regulating primary activation of a TCR complex in either a stimulatory manner or an inhibitory manner. In some embodiments, the intracellular signaling domain of the CAR is involved in inducing T cell activation and/or a cytotoxic response against cells that express the antigen that is bound by the CAR. In some cases, CARs are also referred to as artificial T cell receptors, chimeric immunoreceptors, or chimeric T cell receptors.

The ability of a CAR to bind a target compound (such as BCMA) can be compared with the ability of a negative control CAR to bind the same target compound. A CAR that binds the same target compound using a suitable assay with 25%, 50%, 100%, 200%, 1000% or higher increased affinity relative to the negative control CAR, is said to “specifically bind to” or “specifically interact with” the target compound. Suitable assays include flow cytometry, Surface Plasmon Resonance (SPR) technology/BIACORE instrument or Kinetic Exclusion Assay (KinExA®).”

As used herein, the term “heterodimeric receptor” includes any receptor which is a macromolecular complex formed by two protein monomers which are different to each other. The term may further be understood to include functional heterodimeric fragments or parts of receptors. As non-limiting examples, the term includes a signal transduction moiety of a B-cell receptor (which is an Ig-α/Ig-β heterodimer (CD79)), B-cell receptor heavy and light chain, a Toll-like receptor 1 and 2 heterodimer, an integrin like αvβ5, a phagocytic receptor Mac-1, an MHC, a CD94, a T-cell receptor (TCR), an alpha beta (αβ) TCR, a gamma delta (γδ) TCR, and any other receptor or functional fragment or part thereof that may occur as a heterodimer.

An “antigen” is a molecule or molecular structure that an antigen receptor or an antigen-binding protein can recognize (for example, bind to). An antigen can be or can comprise, for example, a peptide, a polypeptide, a carbohydrate, a chemical, a moiety, a non-peptide antigen, a phosphoantigen, a tumor-associated antigen, a neoantigen, a tumor microenvironment antigen, a microbial antigen, a viral antigen, a bacterial antigen, an autoantigen, a glycan-based antigen, a peptide-based antigen, a lipid-based antigen, or any combination thereof. In some embodiments, an antigen is capable of inducing an immune response. In some examples, an antigen binds to an antigen receptor or antigen-binding protein, or induces an immune response, when present in a complex e.g., presented by MHC. In some cases, an antigen adopts a certain conformation in order to bind to an antigen receptor or antigen-binding protein, and/or to induce an immune response, e.g., adopts a conformation in response to the presence or absence of one or more metabolites. Antigen can refer to a whole target molecule, a whole complex, a or a fragment of a target molecule or complex that binds to an antigen receptor or an antigen-binding protein. Antigen receptors that recognize antigens include exogenous antigen-recognition receptors disclosed herein and other antigen-recognition receptors, such as endogenous T cell receptors.

A “TEG” is a T cell engineered, or genetically modified to express a defined γδ TCR as disclosed herein. In a non-limiting example, a TEG can be an alpha-beta T cell (e.g., αβ T-cell) that is engineered to express a defined γδ TCR.

A “genetically modified” or “modified cell” cell refers to a cell in which the nuclear, organellar or extrachromosomal nucleic acid sequences of a cell has been transformed, modified or transduced using recombinant DNA technology to comprise a heterologous nucleic acid molecule, and is used interchangeably with “engineered cell,” “transformed cell,” and “transduced cell.” In one aspect, a genetically modified cell as disclosed herein expresses a protein encoded by a nucleic acid molecule engineered in such manner to contain an insertion of at least one nucleotide, a deletion of at least one nucleotide, and/or a substitution of at least one nucleotide in a sequence encoding at least one heterologous protein.

The term “heterologous” refers to an entity that is not native to the cell or species of interest.

The terms “polypeptide” and “protein” are used interchangeably to refer to a polymer of amino acid residues.

As used herein, the terms “target site”, “target sequence”, or “nucleic acid locus” refer to a nucleic acid sequence that defines a portion of a nucleic acid sequence to be modified or edited and to which a homologous recombination composition is engineered to target.

The terms “upstream” and “downstream” refer to locations in a nucleic acid sequence relative to a fixed position. Upstream refers to a position in a sequence that is 5′ (i.e., nearer the 5′ end of the strand) relative to the fixed position, and downstream refers to the region that is 3′ (i.e., nearer the 3′ end of the strand) relative to the fixed position.

As used herein, the term “or” is generally employed in its sense including “and/or” unless the content clearly dictates otherwise.

As used herein, the disclosure of numerical ranges by numerical endpoints includes all numbers encompassed by that range (e.g., “1 to 5” includes but is not limited to 1, 1.25, 1.5, 1.75, 2, 2.3, 2.5, 2.8, 3, 3.1, 3.3, 3.8, 3.9, 4, 4.25, 4.5, 4.75 and 5). Unless otherwise indicated, all numbers used herein to express quantities, amounts, dimensions, measurements, and the like should be understood as encompassing the specific quantities, amounts, dimensions, measurements and so on, and also as encompassing such instances modified by the term “about.” Accordingly, unless indicated to the contrary, the numerical descriptions set forth herein may vary while remaining well within the teachings of the present disclosure. At the very least, each numerical value should be construed in view of the number of significant digits and by applying routine rounding techniques. As various changes could be made in the above-described cells and methods without departing from the scope of the invention, it is intended that all matter contained in the above description and in the examples given below, shall be interpreted as illustrative and not in a limiting sense.

### II. Compositions

The current disclosure encompasses various compositions which may comprise, in the alternative or in any combination: polynucleotides; vectors; polypeptides encoded by the polynucleotides and/or vectors; cells (or populations of cells) comprising the polynucleotides and/or vectors; cells expressing the polypeptides; and pharmaceutical compositions comprising any one or more polynucleotides, vectors, polypeptides, and/or cells as described herein, for use as a medicament, or in a method of treatment comprising administering such compositions to a subject in need thereof. In one aspect the compositions can be used for the treatment of a cancer as detailed further herein below.

### A. Polynucleotides

One aspect of the present disclosure encompasses polynucleotides comprising nucleic acids encoding each of the monomers of a heterodimeric γδT-cell receptor or functional fragments thereof, and at least one nucleic acid encoding a chimeric antigen receptor (CAR), wherein the nucleic acids are combined in an expression construct and operably linked to the same promoter. In some aspects, the polynucleotide further comprises one or more cis-acting regulatory sequences.

The present inventors have surprisingly found that the expression of a CAR by an immune cell may be significantly enhanced when the cell comprises a polynucleotide which comprises a nucleic acid encoding a CAR polypeptide, and nucleic acid sequences encoding each of the monomers of a heterodimeric γδT-cell receptor or functional fragments thereof, and when the nucleic acids are operably linked to the same promoter sequence.

Improved expression of a CAR may be assessed by any standard technique available to the skilled person such as flow cytometry, FACS, and the like. Further non-limiting examples are provided in the experimental section.

Improved expression of a CAR may result in an improvement of a biological parameter and/or function of a cell expressing the receptor, which can be or can comprise, for example, cellular proliferation, cellular survival, magnitude of immune effector function, duration of immune effector function, cytotoxic effects on a target cell (e.g., a cancer cell), production of inflammatory mediators, an anti-cancer immune response, cellular differentiation, cellular dedifferentiation, and the like, as discussed earlier herein. The improvement may be at least 5%, at least 10%, at least 20%, at least 30%, at least 40%, at least 50%, at least 60%, at least 70%, at least 80%, at least 90%, at least 2-fold, at least 3-fold, at least 5-fold, at least 10-fold, at least 20-fold, at least 50-fold, at least 100-fold, or at least 1000-fold compared to a cell which expresses the receptor without at least one nucleic acid encoding a polypeptide other than the monomers of the receptor having been inserted in the polynucleotide encoding the receptor, between the nucleic acids encoding the receptor monomers. Assays to measure these biological parameters and/or functions are described elsewhere herein and further non-limiting examples are provided in the experimental section.

In some aspects a polynucleotide encompassed by the current disclosure is multicistronic. In other aspects, a polynucleotide can be tricistronic or tetracistronic. In another exemplary aspect, the polynucleotide is a tricistronic polynucleotide comprising a first nucleic acid encoding a chimeric antigen receptor, a second nucleic acid encoding a γ monomer of γδT-cell receptor or functional fragments or variants thereof, and a third nucleic acid encoding a δ monomer of a γδT-cell receptor or functional fragments or variants thereof and wherein the first, second and third nucleic acids are operably linked to the same promoter. In another non-limiting example, the polynucleotide is a tetracistronic polynucleotide comprising a first nucleic acid encoding a chimeric antigen receptor, a second nucleic acid encoding a γ monomer of γδT-cell receptor or functional fragments or variant thereof, and a third nucleic acid encoding a δ monomer of a γδT-cell receptor or functional fragments thereof or a variant thereof and a fourth nucleic acid encoding additional functionally relevant polypeptides and wherein the first, second, third and fourth nucleic acids are operably linked to the same promoter. In some aspects the polypeptide is multicistronic and comprises at least one nucleic acid encoding a chimeric antigen receptor and a second nucleic acid encoding at least a monomer or functional fragment thereof of a heterodimeric γδT-cell receptor.

The nucleic acids encoding each of the monomers of the heterodimeric receptor may preferably be in a specific order with respect to their positions in the polynucleotide. The term “specific order” as used herein refers to the order in which the encoded polypeptides are translated from the multicistronic mRNA by the ribosome. In other words, it refers to the positions of the nucleic acids encoding the polypeptides in the multicistronic polynucleotide. Thus, a nucleic acid that is located at the “first position” is understood to be the first nucleic acid to be expressed, and a nucleic acid that is located at the “last position” is understood to be the last nucleic acid to be expressed. As a non-limiting example, in a polynucleotide encoding a γδT-cell receptor as described herein, an order of the nucleic acids may be: γ chain-encoding nucleic acid (first position), followed by a nucleic acid encoding a CAR, followed by a δ chain-encoding nucleic acid (last position). Alternatively, the order of nucleic acids may be: δ chain-encoding nucleic acid (first position), followed by a nucleic acid encoding a CAR, followed by a γ chain-encoding nucleic acid (last position). Preferably, the polynucleotides described above are tricistronic or tetracistronic.

In some embodiments a polynucleotide encoding a γδT-cell receptor is tricistronic, and comprises a nucleic acid encoding a γ-chain at the first position and a nucleic acid encoding a δ-chain at the third position.

In some embodiments a polynucleotide encoding a γδT-cell receptor is tricistronic, and comprises a nucleic acid encoding a δ-chain at the first position and a nucleic acid encoding a γ-chain at the third position.

In some embodiments a polynucleotide encoding a γδT-cell receptor is tetracistronic, and comprises a nucleic acid encoding a γ-chain at the first position and a nucleic acid encoding a δ-chain at the fourth position.

In some embodiments a polynucleotide encoding a γδT-cell receptor is tetracistronic, and comprises a nucleic acid encoding a δ-chain at the first position and a nucleic acid encoding a γ-chain at the fourth position.

In various different aspects, within a polycistronic (multicistronic) construct, the nucleic acids (cistrons) encoding each of the γ monomer of γδT-cell receptor, the δ monomer of γδT-cell receptor, and the CAR can be variously positioned relative to one another. Put differently, for example, the nucleic acid encoding the γ monomer of γδT-cell receptor, can be placed upstream or downstream in the polynucleotide with respect to the nucleic acid(s) encoding the δ monomer of γδT-cell receptor, and with respect to the nucleic acid encoding the CAR, or with respect to other nucleic acids in the construct.

In a preferred non-limiting example, the order of the nucleic acids in the construct can be, starting from the first position and following in successive positions, in order: (i) a nucleic acid encoding γ-chain/s and derivatives, variations or fragments thereof; (ii) a nucleic acid encoding a CAR; and (iii) a nucleic acid encoding a δ-chain/s and derivatives, variations or fragments thereof. In another non-limiting example, the order of the nucleic acids in the construct can be: (i) a nucleic acid encoding a γ-chain/s and derivatives, variations or fragments thereof; (ii) a nucleic acid encoding a δ-chain/s and derivatives, variations or fragments thereof; and (iii) a nucleic acid encoding a CAR. In another non-limiting example, the order of the nucleic acids in the construct can be: (i) a nucleic acid encoding a δ-chain/s and derivatives, variations or fragments thereof; (ii) a nucleic acid encoding a γ-chain/s and derivatives, variations or fragments thereof; and a nucleic acid encoding a CAR. In another non-limiting example, the order of the nucleic acids in the construct can be: (i) a nucleic acid encoding a CAR; (ii) a nucleic acid encoding a γ-chain/s and derivatives, variations or fragments thereof; and (iii) a nucleic acid encoding a δ-chain/s and derivatives, variations or fragments thereof. In yet another non-limiting example, the order of the nucleic acids in the construct can be: (i) a nucleic acid encoding a CAR; (ii) a nucleic acid encoding a δ-chain/s and derivatives, variations or fragments thereof; and (iii) a nucleic acid encoding a γ-chain/s and derivatives, variations or fragments thereof. Any of the constructs optionally further comprise cis-acting regulatory components present at one or more locations. It should be understood that the current disclosure encompasses all variations in the order of cistrons in a multicistronic construct, such that the expression of the CAR is maximized, and/or the desired biological function or effect as described herein of the cell or cell population is enhanced.

Further, in various aspects, the present disclosure encompasses a polynucleotide comprising A or B, wherein: (A) is a nucleic acid represented by (i)-(ii)-(iii), wherein: (i) is a nucleic acid encoding a γ chain of a γδT-cell receptor or a functional fragment thereof, (ii) is at least one nucleic acid encoding a CAR or a functional fragment thereof, and; (iii) is a nucleic acid encoding a δ chain of a γδT-cell receptor or a functional fragment thereof, preferably wherein (ii) is positioned between (i) and (iii), is positioned downstream of both (i) and (iii), or is positioned upstream of both (i) and (iii); and wherein (B) is a nucleic acid represented by (iv)-(v)-(vi), wherein: (iv) is a nucleic acid encoding a δ chain of a γδT-cell receptor or a functional fragment thereof, (v) is at least one nucleic acid encoding a CAR or a functional fragment thereof, and; (vi) is a nucleic acid encoding a γ chain of a γδT-cell receptor or a functional fragment thereof, wherein (v) is positioned between (iv) and (vi), is positioned downstream of both (iv) and (vi), or is positioned upstream of both (iv) and (vi).

In a polynucleotide comprising A or B, (i) and (vi) can be nucleic acids comprising a nucleotide sequence encoding a polypeptide having at least 60%, 70%, 80%, 90%, 95%, or 100% identity or similarity with an amino acid sequence selected from SEQ ID NOs: 35, 36, 37, 39, 41, 43, 44, 45, 46, 51, 54, 56, 58, 60, 62, 63, 65, 67, 69, 71, 73, 75, 77, 79, 80, 82, 86, 87, 90, 91, 94, 95, 98, 99, 101, 102, 125, 126, 129, 130, 133, 134, 137, 138, 141, 142, 145, 146, 149, and 150 preferably selected from SEQ ID NOs: 35, 36, 37, 44, 45, 46, 51, 63, 65, 67, 69, 77, 80, 86, 87, 90, 91, 94, 95, 98, 99, 101, 102, 125, 126, 129, 130, 133, 134, 137, 138, 141, 142, 145, 146, 149, and 150 and more preferably selected from SEQ ID Nos: 35, 36, 37, 44, 45, 46, 51, 63, 65, 67, 69, 77, 80, 86, 90, 94, 98, 125, 129, 133, 137, 141, 145, and 149 and/or (iii) and (iv) are nucleic acids comprising a nucleotide sequence encoding a polypeptide having at least 60%, 70%, 80%, 90%, 95%, or 100% identity or similarity with an amino acid sequence selected from SEQ ID NOs: 32, 33, 34, 38, 40, 42, 47, 48, 49, 50, 52, 53, 55, 57, 59, 61, 64, 66, 68, 70, 72, 74, 76, 78, 81, 83, 84, 85, 88, 89, 92, 93, 96, 97, 100, 123, 124, 127, 128, 131, 132, 135, 136, 139, 140, 143, 144, 147, and 148 preferably selected from SEQ ID NOs: 32, 33, 34, 40, 47, 48, 49, 50, 52, 61, 64, 66, 68, 76, 81, 84, 85, 88, 89, 92, 93, 96, 97, 100, 123, 124, 127, 128, 131, 132, 135, 136, 139, 140, 143, 144, 147, and 148, more preferably selected from SEQ ID NOs: 33, 34, 47, 48, 49, 50, 52, 55, 57, 59, 61, 64, 66, 68, 76, 81, 85, 88, 92, 96, 123, 127, 131, 135, 139, 143, and 147.

In further aspects, in a polynucleotide comprising A or B, (i) and (vi) can be nucleic acids comprising a nucleotide sequence encoding a polypeptide having at least 60%, 70%, 80%, 90%, 95%, or 100% identity or similarity with an amino acid sequence of SEQ ID NO: 37. In further aspects, (i) and (vi) can be nucleic acids comprising a nucleotide sequence encoding a polypeptide comprising an amino acid sequence of SEQ ID NO: 37. In various aspects, (i) and (vi) can be nucleic acids comprising a nucleotide sequence encoding a polypeptide having at least 60%, 70%, 80%, 90%, 95%, or 100% identity or similarity with GITISATS (SEQ ID NO: 166). In various aspects, (i) and (vi) can be nucleic acids comprising a nucleotide sequence encoding a polypeptide comprising an amino acid sequence of GITISATS (SEQ ID NO: 166). In various aspects, (i) and (vi) can be nucleic acids comprising a nucleotide sequence encoding a polypeptide having at least 60%, 70%, 80%, 90%, 95%, or 100% identity or similarity with ISYDGTV (SEQ ID NO: 167). In various aspects, (i) and (vi) can be nucleic acids comprising a nucleotide sequence encoding a polypeptide comprising an amino acid sequence of ISYDGTV (SEQ ID NO: 167). In various aspects, (i) and (vi) can be nucleic acids comprising a nucleotide sequence encoding a polypeptide comprising SEQ ID NO: 37 and a nucleotide sequence encoding a polypeptide comprising GITISATS (SEQ ID NO: 166) or ISYDGTV (SEQ ID NO: 167). In various aspects, (i) and (vi) can be nucleic acids comprising a nucleotide sequence encoding a polypeptide comprising SEQ ID NO: 37, a nucleotide sequence encoding a polypeptide comprising GITISATS (SEQ ID NO: 166) and a nucleotide sequence encoding a polypeptide comprising ISYDGTV (SEQ ID NO: 167). In various aspects, (i) and (vi) can be nucleic acids comprising a nucleotide sequence encoding a polypeptide having at least 60%, 70%, 80%, 90%, 95%, or 100% identity or similarity with SEQ ID NO: 41. In various aspects, (i) and (vi) can be nucleic acids comprising a nucleotide sequence encoding a polypeptide comprising SEQ ID NO: 41.

In further aspects, in a polynucleotide comprising A or B, (iii) and (iv) are nucleic acids comprising a nucleotide sequence encoding a polypeptide having at least 60%, 70%, 80%, 90%, 95%, or 100% identity or similarity with an amino acid sequence of SEQ ID NO: 34. In further aspects, (iii) and (iv) are nucleic acids comprising a nucleotide sequence encoding a polypeptide comprising an amino acid sequence of SEQ ID NO: 34. In various aspects, (iii) and (iv) can be nucleic acids comprising a nucleotide sequence encoding a polypeptide having at least 60%, 70%, 80%, 90%, 95%, or 100% identity or similarity with GEAIGNYY SEQ ID NO: 168). In various aspects, (iii) and (iv) can be nucleic acids comprising a nucleotide sequence encoding a polypeptide comprising an amino acid sequence of GEAIGNYY SEQ ID NO: 168). In various aspects, (iii) and (iv) can be nucleic acids comprising a nucleotide sequence encoding a polypeptide having at least 60%, 70%, 80%, 90%, 95%, or 100% identity or similarity with an amino acid sequence of EKD. In various aspects, (iii) and (iv) can be nucleic acids comprising a nucleotide sequence encoding a polypeptide comprising an amino acid sequence of EKD. In various aspects, (iii) and (iv) can be nucleic acids comprising a nucleotide sequence encoding a polypeptide comprising SEQ ID NO: 34 and a nucleotide sequence encoding a polypeptide comprising GEAIGNYY SEQ ID NO: 168) or EKD. In various aspects (iii) and (iv) can be nucleic acids comprising a nucleotide sequence encoding a polypeptide comprising SEQ ID NO: 34, a nucleotide sequence encoding a polypeptide comprising GEAIGNYY SEQ ID NO: 168) and a nucleotide sequence encoding a polypeptide comprising EKD. In various aspects, (iii) and (iv) can be nucleic acids comprising a nucleotide sequence encoding a polypeptide having at least 60%, 70%, 80%, 90%, 95%, or 100% identity or similarity with SEQ ID NO: 40. In various aspects (iii) and (iv) can be nucleic acids comprising a nucleotide sequence encoding a polypeptide comprising SEQ ID NO: 40.

In further aspects, in a polynucleotide comprising A or B, (i) and (vi) can be nucleic acids comprising a nucleotide sequence encoding a polypeptide comprising an amino acid sequence selected from SEQ ID NO: 37, 166 (GITISATS), 167 (ISYDGTV), and combinations thereof; and (iii) and (iv) are nucleic acids comprising a nucleotide sequence encoding a polypeptide comprising an amino acid sequence selected from SEQ ID NO: 34, 168 (GEAIGNYY), EKD and combinations thereof. In various aspects, (i) and (vi) can be nucleic acids comprising a nucleotide sequence encoding a polypeptide comprising SEQ ID NO: 41 and iii) and (iv) can be nucleic acids comprising a nucleotide sequence encoding a polypeptide comprising SEQ ID NO: 40.

In further aspects, in a polynucleotide comprising A or B, (i) and (vi) can be nucleic acids comprising a nucleotide sequence encoding a polypeptide having at least 60%, 70%, 80%, 90%, 95%, or 100% identity or similarity with an amino acid sequence of SEQ ID NO: 51. In further aspects, (i) and (vi) can be nucleic acids comprising a nucleotide sequence encoding a polypeptide comprising an amino acid sequence of SEQ ID NO: 51. In various aspects, (i) and (vi) can be nucleic acids comprising a nucleotide sequence encoding a polypeptide having at least 60%, 70%, 80%, 90%, 95%, or 100% identity or similarity with EGSTGY (SEQ ID NO: 169). In various aspects, (i) and (vi) can be nucleic acids comprising a nucleotide sequence encoding a polypeptide comprising an amino acid sequence of EGSTGY (SEQ ID NO: 169). In various aspects, (i) and (vi) can be nucleic acids comprising a nucleotide sequence encoding a polypeptide having at least 60%, 70%, 80%, 90%, 95%, or 100% identity or similarity with YDSYTSSV (SEQ ID NO: 170). In various aspects, (i) and (vi) can be nucleic acids comprising a nucleotide sequence encoding a polypeptide comprising an amino acid sequence of YDSYTSSV (SEQ ID NO: 170). In various aspects, (i) and (vi) can be nucleic acids comprising a nucleotide sequence encoding a polypeptide comprising SEQ ID NO: 51 and a nucleotide sequence encoding a polypeptide comprising EGSTGY (SEQ ID NO: 169) or YDSYTSSV (SEQ ID NO: 170). In various aspects, (i) and (vi) can be nucleic acids comprising a nucleotide sequence encoding a polypeptide comprising SEQ ID NO: 51, a nucleotide sequence encoding a polypeptide comprising EGSTGY (SEQ ID NO: 169) and a nucleotide sequence encoding a polypeptide comprising YDSYTSSV (SEQ ID NO: 170). In various aspects, (i) and (vi) can be nucleic acids comprising a nucleotide sequence encoding a polypeptide having at least 60%, 70%, 80%, 90%, 95%, or 100% identity or similarity with SEQ ID NO: 62. In various aspects, (i) and (vi) can be nucleic acids comprising a nucleotide sequence encoding a polypeptide comprising SEQ ID NO: 62.

In further aspects, in a polynucleotide comprising A or B, (iii) and (iv) are nucleic acids comprising a nucleotide sequence encoding a polypeptide having at least 60%, 70%, 80%, 90%, 95%, or 100% identity or similarity with an amino acid sequence of SEQ ID NO: 52. In further aspects, (iii) and (iv) are nucleic acids comprising a nucleotide sequence encoding a polypeptide comprising an amino acid sequence of SEQ ID NO: 52. In various aspects, (iii) and (iv) can be nucleic acids comprising a nucleotide sequence encoding a polypeptide having at least 60%, 70%, 80%, 90%, 95%, or 100% identity or similarity with NSMFDY (SEQ ID NO: 171). In various aspects, (iii) and (iv) can be nucleic acids comprising a nucleotide sequence encoding a polypeptide comprising an amino acid sequence of NSMFDY SEQ ID NO: 171). In various aspects, (iii) and (iv) can be nucleic acids comprising a nucleotide sequence encoding a polypeptide having at least 60%, 70%, 80%, 90%, 95%, or 100% identity or similarity with an amino acid sequence of ISSIKDK (SEQ ID NO: 172). In various aspects, (iii) and (iv) can be nucleic acids comprising a nucleotide sequence encoding a polypeptide comprising an amino acid sequence of ISSIKDK (SEQ ID NO: 172). In various aspects, (iii) and (iv) can be nucleic acids comprising a nucleotide sequence encoding a polypeptide comprising SEQ ID NO: 52 and a nucleotide sequence encoding a polypeptide comprising NSMFDY (SEQ ID NO: 171) or ISSIKDK (SEQ ID NO: 172). In various aspects (iii) and (iv) can be nucleic acids comprising a nucleotide sequence encoding a polypeptide comprising SEQ ID NO: 52, a nucleotide sequence encoding a polypeptide comprising NSMFDY SEQ ID NO: 171) and a nucleotide sequence encoding a polypeptide comprising ISSIKDK (SEQ ID NO: 172). In various aspects, (iii) and (iv) can be nucleic acids comprising a nucleotide sequence encoding a polypeptide having at least 60%, 70%, 80%, 90%, 95%, or 100% identity or similarity with SEQ ID NO: 61. In various aspects (iii) and (iv) can be nucleic acids comprising a nucleotide sequence encoding a polypeptide comprising SEQ ID NO: 61.

In further aspects, in a polynucleotide comprising A or B, (i) and (vi) can be nucleic acids comprising a nucleotide sequence encoding a polypeptide comprising an amino acid sequence selected from SEQ ID NOs: 51, 169, 170, and combinations thereof; and (iii) and (iv) are nucleic acids comprising a nucleotide sequence encoding a polypeptide comprising an amino acid sequence selected from SEQ ID NO: 52, 171, 172 and combinations thereof. In various aspects, (i) and (vi) can be nucleic acids comprising a nucleotide sequence encoding a polypeptide comprising SEQ ID NO: 62 and iii) and (iv) can be nucleic acids comprising a nucleotide sequence encoding a polypeptide comprising SEQ ID NO: 61.

i. Chimeric Antigen Receptors

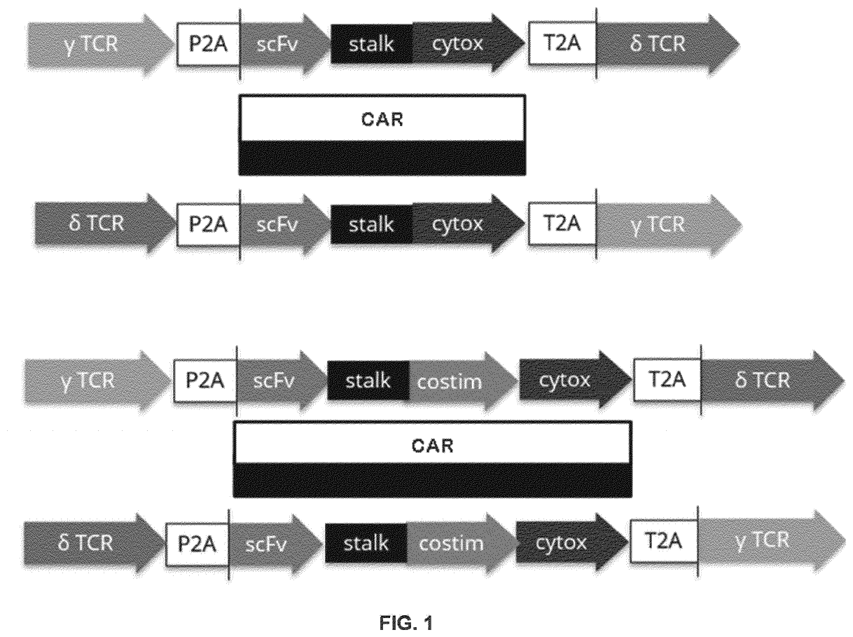

The term “chimeric antigen receptor” or “CAR” or “chimeric immunoreceptors”, “chimeric T cell receptors” or “artificial T cell receptors” are recombinant receptors comprising one or more target antigen binding domains and an intracellular signaling domain. In some aspects the current disclosure encompasses use of any CAR sequence known in the art. In some aspects the CAR may comprise an antigen recognition domain (extracellular target recognition domain), an extracellular hinge or stalk region, a transmembrane domain, one or more co-stimulatory domains and a cytotoxicity induction domain. FIG. 1 is a schematic illustration of exemplary polycistronic constructs used to express monomers of a heterodimeric γδ T-cell receptor or functional fragments thereof, and a chimeric antigen receptor (CAR).

### Antigen Binding/Recognition Domain

As used herein the terms “antigen binding domain,” “antigen recognition domain” and “target recognition domain” may be used interchangeably. Non-limiting examples of a CAR targeting domain may be derived from a monoclonal antibody, a polyclonal antibody, a recombinant antibody, a human antibody, a humanized antibody, or a functional derivative, variant or fragment thereof, including, but not limited to, a Fab, a Fab′, a F(ab′)2, an Fv, a single-chain Fv (scFv), minibody, a diabody, and a single-domain antibody such as a heavy chain variable domain (VH), a light chain variable domain (VL), a DARPin, a monobody, a nanobody, an affibody, a non-antibody domain, and any combination thereof. A non-antibody CAR targeting domain can be from or derived from a receptor or a receptor ligand, for example, APRIL can be used to target BCMA.

In some aspects the antigen binding domain may selectively bind any tumor antigens well known in the art. Example include but are not limited to glioma associated antigen, carcinoembryonic antigen (CEA), EGFRvIII, Interleukin-11 receptor alpha (IL-11Ra), Interleukin-13 receptor subunit alpha-2 (IL-13Ra or CD213A2), epidermal growth factor receptor (EGFR), B7H3 (CD276), Kit (CD117), carbonic anhydrase (CA-IX), CS-1 (also referred to as CD2 subset 1), Mucin 1, cell surface associated (MUC1), B cell maturation antigen (BCMA), oncogene fusion protein consisting of breakpoint cluster region (BCR) and Abelson murine leukemia viral oncogene homolog 1 (Abl) bcr-abl, Receptor tyrosine-protein kinase ERBB2 (HER2/neu), β-human chorionic gonadotropin, alphafetoprotein (AFP), anaplastic lymphoma kinase (ALK), CD19, CD123, cyclin B1, lectin-reactive AFP, Fos-related antigen 1, adrenoceptor beta 3 (ADRB3), thyroglobulin, tyrosinase; ephrin type-A receptor 2 (EphA2), Receptor for Advanced Glycation Endproducts (RAGE-1), renal ubiquitous 1 (RU1), renal ubiquitous 2 (RU2), synovial sarcoma, X breakpoint 2 (SSX2), A kinase anchor protein 4 (AKAP-4), lymphocyte-specific protein tyrosine kinase (LCK), proacrosin binding protein sp32 (OY-TES1), Paired box protein Pax-5 (PAX5), Squamous Cell Carcinoma Antigen Recognized By T Cells 3 (SART3), C-type lectin-like molecule-1 (CLL-1 or CLECL1), fucosyl GM1, hexasaccharide portion of globoH glycoceramide (GloboH), MN-CA IX, Epithelial cell adhesion molecule (EPCAM), EVT6-AML, transglutaminase 5 (TGS5), human telomerase reverse transcriptase (hTERT), polysialic acid, placenta-specific 1 (PLAC1), intestinal carboxyl esterase, LewisY antigen, sialyl Lewis adhesion molecule (sLe), lymphocyte antigen 6 complex, locus K 9 (LY6K), heat shock protein 70-2 mutated (mut hsp70-2), M-CSF, v-myc avian myelocytomatosis viral oncogene neuroblastoma derived homolog (MYCN), Ras Homolog Family Member C (RhoC), Tyrosinaserelated protein 2 (TRP-2), Cytochrome P450 1B1 (CYP1B1), CCCTC-Binding Factor (Zinc Finger Protein)-Like (BORIS or Brother of the Regulator of Imprinted Sites), prostase, prostate-specific antigen (PSA), paired box protein Pax-3 (PAX3), prostatic acid phosphatase (PAP), Cancer/testis antigen 1 (NY-ESO-1), Cancer/testis antigen 2 (LAGE-1a), LMP2, neural cell adhesion molecule (NCAM), tumor protein p53 (p53), p53 mutant, Rat sarcoma (Ras) mutant, glycoprotein 100 (gp100), prostein, OR51E2, pannexin 3 (PANX3), prostate-specific membrane antigen (PSMA), prostate stem cell antigen (PSCA), high molecular weight-melanoma-associated antigen (HMWMAA), Hepatitis A virus cellular receptor 1 (HAVCR1), vascular endothelial growth factor receptor 2 (VEGFR2), Platelet-derived growth factor receptor beta (PDGFR-beta), legumain, human papilloma virus E6 (HPV E6), human papilloma virus E7 (HPV E7), survivin, telomerase, sperm protein 17 (SPA17), Stage-specific embryonic antigen-4 (SSEA-4), tyrosinase, TCR Gamma Alternate Reading Frame Protein (TARP), Wilms tumor protein (WT1), prostate-carcinoma tumor antigen-1 (PCTA-1), melanoma inhibitor of apoptosis (ML-IAP), MAGE, Melanoma-associated antigen 1 (MAGE-A1), melanoma cancer testis antigen-1 (MAD-CT-1), melanoma cancer testis antigen-2 (MAD-CT-2), melanoma antigen recognized by T cells 1 (MelanA/MART1), X Antigen Family, Member 1A (XAGE1), elongation factor 2 mutated (ELF2M), ERG (TMPRSS2 ETS fusion gene), N-Acetyl glucosaminyl-transferase V (NA17), neutrophil elastase, sarcoma translocation breakpoints, mammary gland differentiation antigen (NY-BR-1), ephrinB2, CD20, CD22, CD24, CD30, CD33, CD38, CD44v6, CD97, CD171, CD179a, androgen receptor, insulin growth factor (IGF)-I, IGF-II, IGF-I receptor, ganglioside GD2 (GD2), o-acetyl-GD2 ganglioside (OAcGD2), ganglioside GD3 (aNeu5Ac (2-8) aNeu5Ac (2-3) bDGalp (1-4) bDGlcp (1-1) Cer), ganglioside GM3 (aNeu5Ac (2-3) bDGalp (1-4) bDGlcp (1-1) Cer), G protein-coupled receptor class C group 5, member D (GPRC5D), G protein-coupled receptor 20 (GPR20), chromosome X open reading frame 61 (CXORF61), folate receptor (FRa), folate receptor beta, Receptor tyrosine kinase-like orphan receptor 1 (ROR1), Fms-Like Tyrosine Kinase 3 (Flt3), Tumor-associated glycoprotein 72 (TAG72), Tn antigen (TN Ag or (GalNAca-Ser/Thr)), angiopoietin-binding cell surface receptor 2 (Tie 2), tumor endothelial marker 1 (TEM1 or CD248), tumor endothelial marker 7-related (TEM7R), claudin 6 (CLDN6), thyroid stimulating hormone receptor (TSHR), uroplakin 2 (UPK2), mesothelin, Protease Serine 21 (Testisin or PRSS21), epidermal growth factor receptor (EGFR), fibroblast activation protein alpha (FAP), Olfactory receptor 51E2 (OR51E2), ETS translocation-variant gene 6, located on chromosome 12p (ETV6-AML), CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1). In some aspects the antigen binding domain is selective for BCMA, CD19, PSA, HER-2/neu, MUC1, Carcinoembryonic antigen (CEA), CA-125, Epithelial tumor antigen (ETA), Tyrosinase, Melanoma-associated antigen (MAGE).

Some exemplary antigen binding domain target sequences are provided in Table A1. In some aspects the antigen binding domain target sequence may comprise a sequence which has at least 60 to 100%, or 60, 65, 70, 75, 80, 85, 90, 95, 96, 97, 98, or 99% similarity or identity to any one of SEQ ID NOS: 1-2 and 162. Some exemplary antigen binding domains are provided in Table A2. In some aspects the antigen binding domain may comprise a sequence which has at least 60 to 100%, or 60, 65, 70, 75, 80, 85, 90, 95, 96, 97, 98, or 99% similarity or identity to any one of SEQ ID NOs: 163-164.

TABLE A1
Antigen binding domain Target Sequences:

| SEQ ID | Tumor | Exemplary mono- | |
|---|---|---|---|
| NO | Antigen | clonal antibody | SEQUENCE |
| 1 | BCMA | 139103 (human) | MLQMAGQCSQNEYFDSLLHACIPCQLRCSSNTPPLTCQRYCNASV |
| | | | TNSVKGTNAILWTCLGLSLIISLAVFVLMFLLRKINSEPLKDEFKNTG |
| | | | SGLLGMANIDLEKSRTGDEIILPRGLEYTVEECTCEDCIKSKPKVDS |
| | | | DHCFPLPAMEEGATILVTTKTNDYCKSLPAALSATEIEKSISAR |
| 2 | CD19 | FMC-63 | MPPPRLLFFLLFLTPMEVRPEEPLVVKVEEGDNAVLQCLKGTSDGP |
| | | | TQQLTWSRESPLKPFLKLSLGLPGLGIHMRPLAIWLFIFNVSQQMG |
| | | | GFYLCQPGPPSEKAWQPGWTVNVEGSGELFRWNVSDLGGLGCG |
| | | | LKNRSSEGPSSPSGKLMSPKLYVWAKDRPEIWEGEPPCLPPRDSL |
| | | | NQSLSQDLTMAPGSTLWLSCGVPPDSVSRGPLSWTHVHPKGPKS |
| | | | LLSLELKDDRPARDMWVMETGLLLPRATAQDAGKYYCHRGNLTM |
| | | | SFHLEITARPVLWHWLLRTGGWKVSAVTLAYLIFCLCSLVGILHLQR |
| | | | ALVLRRKRKRMTDPTRRFFKVTPPPGSGPQNQYGNVLSLPTPTSG |
| | | | LGRAQRWAAGLGGTAPSYGNPSSDVQADGALGSRSPPGVGPEE |
| | | | EEGEGYEEPDSEEDSEFYENDSNLGQDQLSQDGSGYENPEDEPL |
| | | | GPEDEDSFSNAESYENEDEELTQPVARTMDFLSPHGSAWDPSRE |
| | | | ATSLGSQSYEDMRGILYAAPQLRSIRGQPGPNHEEDADSYENMDN |
| | | | PDGPDPAWGGGGRMGTWSTR |
| 162 | CEA | CEAM5 | MESPSAPPHRWCIPWQRLLLTASLLTFWNPPTTAKLTIESTPFNVA |
| | | | EGKEVLLLVHNLPQHLFGYSWYKGERVDGNRQIIGYVIGTQQATP |
| | | | GPAYSGREIIYPNASLLIQNIIQNDTGFYTLHVIKSDLVNEEATGQFR |
| | | | VYPELPKPSISSNNSKPVEDKDAVAFTCEPETQDATYLWWVNNQS |
| | | | LPVSPRLQLSNGNRTLTLFNVTRNDTASYKCETQNPVSARRSDSVI |
| | | | LNVLYGPDAPTISPLNTSYRSGENLNLSCHAASNPPAQYSWFVNG |
| | | | TFQQSTQELFIPNITVNNSGSYTCQAHNSDTGLNRTTVTTITVYAEP |
| | | | PKPFITSNNSNPVEDEDAVALTCEPEIQNTTYLWWVNNQSLPVSPR |
| | | | LQLSNDNRTLTLLSVTRNDVGPYECGIQNELSVDHSDPVILNVLYG |
| | | | PDDPTISPSYTYYRPGVNLSLSCHAASNPPAQYSWLIDGNIQQHTQ |
| | | | ELFISNITEKNSGLYTCQANNSASGHSRTTVKTITVSAELPKPSISSN |
| | | | NSKPVEDKDAVAFTCEPEAQNTTYLWWVNGQSLPVSPRLQLSNG |
| | | | NRTLTLFNVTRNDARAYVCGIQNSVSANRSDPVTLDVLYGPDTPIIS |
| | | | PPDSSYLSGANLNLSCHSASNPSPQYSWRINGIPQQHTQVLFIAKIT |
| | | | PNNNGTYACFVSNLATGRNNSIVKSITVSASGTSPGLSAGATVGIMI |
| | | | GVLVGVALI |

TABLE A2
Antigen binding domains

| SEQ ID NO | Tumor Antigen | SEQUENCE |
|---|---|---|
| 163 | BCMA | MALPVTALLLPLALLLHAARPDIVLTQSPPSLAMSLGKRATISCRASESVTILGS |
| | | HLIHWYQQKPGQPPTLLIQLASNVQTGVPARFSGSGSRTDFTLTIDPVEEDDV |
| | | AVYYCLQSRTIPRTFGGGTKLEIKGSTSGSGKPGSGEGSTKGQIQLVQSGPEL |
| | | KKPGETVKISCKASGYTFTDYSINWVKRAPGKGLKWMGWINTETREPAYAYD |
| | | FRGRFAFSLETSASTAYLQINNLKYEDTATYFCALDYSYAMDYWGQGTSVTV |
| | | SS |
| 164 | CD19 | MALPVTALLLPLALLLHAARPDIQMTQTTSSLSASLGDRVTISCRASQDISKYLN |
| | | WYQQKPDGTVKLLIYHTSRLHSCVPSRFSGSGSGTDYSLTISNLEQEDIATYF |
| | | CQQGNTLPYTFGGGTKLEITGGGGSGGGGSGGGGSEVKLQESGPGLVAPS |
| | | QSLSVTCTVSGVSLPDYGVSWIRQPPRKCLEWLGVIWGSETTYYNSALKSRL |
| | | TIIKDNSKSQVFLKMNSLQTDDTAIYYCAKHYYYGGSYAMDYWGQGTSVTVS |
| | | S |
| 165 | CEA | MDMRVPAQLLGLLLLWLRGARCDIQLTQSPSSLSASVGDRVTITCKASQDVG |
| | | TSVAWYQQKPGKAPKLLIYWTSTRHTGVPSRFSGSGSGTDFTFTISSLQPEDI |
| | | ATYYCQQYSLYRSFGQGTKVEIKRGGSGSGGSGSGGSGSEVQLVESGGGV |
| | | VQPGRSLRLSCSASGFDFTTYWMSWVRQAPGKGLEWIGEIHPDSSTINYAPS |
| | | LKDRFTISRDNAKNTLFLQMDSLRPEDTGVYFCASLYFGFPWFAYWGQGTPV |
| | | TVSSAKP |

### Hinge/Stalk Domain

In some aspects the encoded CAR may comprise a stalk domain. The “stalk domain” or “the hinge domain” or “spacer” as used herein comprises the region between the antigen binding domain and the transmembrane domain. The spacer region provides stability for efficient CAR expression and activity and flexibility to access target antigens and helps minimize steric hindrance. The optimal length and sequence of the spacer varies widely based on the particular CAR and target antigen. In some aspects the stalk domain may be an immunoglobulin-based hinge. In some aspects the stalk domain may not be based on an immunoglobulin hinge. In some aspects the stalk sequence may be a sequence from a human protein, a fragment thereof, or a short oligo- or polypeptide linker. In some aspects the stalk sequence may be derived from a non-human protein. In some aspects stalk domain may be an artificially designed sequence. In some aspects the encoded stalk domain may be 10-300 amino acids in length, or about 10 to about 300 amino acids in length. In some aspects the length of the stalk domain may range from between 10 to 25, or 25 to 50, or 50 to 75, or 75 to 100, or 100 to 125, or 125 to 150, 150 to 175, 175 to 200, 200 to 225, or 225 to 250, or 250 to 275, or 275-300 and intermediate number of amino acids. As described herein, the hinge may extend less than 20, 15, or 10 nanometers from the surface of the cytotoxic cell. Thus, suitability for a stalk can be influenced by both linear lengths, the number of amino acid residues and flexibility of the hinge.

Non-limiting examples of stalk domains include a CD8 and CD28 stalk as provided in Table B. In some aspects of the current disclosure the encoded stalk domain may comprise a sequence which has at least 70, 75, 80, 85, 90, 95, 96, 97, 98, or 99% similarity or identity to SEQ ID NOS: 3-4.

TABLE B
Stalk sequences

| SEQ ID NO | Name | SEQUENCE |
|---|---|---|
| 3 | CD8 stalk | TTTPAPRPPTPAPTIASQPLSLRPEACRPAAGGAVHTRGLDFACD |
| 4 | CD28 stalk | KIEVMYPPPYLDNEKSNGTIIHVKGKHLCPSPLFPGPSKP |

### Transmembrane Domain

In some aspects the encoded CAR may comprise a transmembrane domain that is attached to the extracellular domain. The transmembrane domain sequence may be derived from a natural or recombinant source. Where the source is natural, the domain may be derived from any membrane-bound or transmembrane protein. In one aspect the transmembrane domain is capable of signaling to the intracellular domain(s) whenever the CAR has bound to a target. Non-limiting examples of transmembrane domains include the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, CD154 and fragments thereof. As described herein, exemplary transmembrane domain may include at least the transmembrane region(s) and fragments thereof of, e.g., KIRDS2, OX40, CD2, CD27, LFA-1 (CD11a, CD18), ICOS (CD278), 4-1BB (CD137), GITR, CD40, BAFFR, HVEM (LIGHTR), SLAMF7, CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD 162), LTBR, PAG/Cbp, and CD19. Alternatively, the transmembrane domain may be synthetic, in which case it will comprise predominantly hydrophobic residues. In some aspects the transmembrane domain may further include one or more additional amino acid sequences including but not limited to one or more amino acids associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). Optionally, a short oligo- or polypeptide linker, preferably between 2 and 10 amino acids in length may form the linkage between the transmembrane domain and the intracellular T cell signaling domain and/or T cell costimulatory domain of the CAR. An exemplary linker sequence includes one or more glycine-serine doublets.

In some exemplary aspects the transmembrane domain may be selected from the transmembrane regions of CD8, CD137, CD28 as provided in Table C. In some aspects of the current disclosure the encoded transmembrane domain may comprise a sequence which has at least 70, 75, 80, 85, 90, 95, 96, 97, 98, or 99% identity or similarity to SEQ ID NOS: 5-7.

TABLE C
Transmembrane sequences

| SEQ | | |
|---|---|---|
| ID | | |
| NO | Name | SEQUENCE |
| 5 | CD8 trans- | IYIWAPLAGTCGVLLLSLVITLYC |
| | membrane | |
| 6 | CD137 (4-1BB) | IISFFLALTSTALLFLLFFLTLRFSVV |
| | transmembrane | |
| 7 | CD28 trans- | FWVLVVVGGVLACYSLLVTVAFIIFWV |
| | membrane | |

### Intracellular Region

In some aspects the CAR may include one or more intracellular T cell signaling domains for activation of at least one of the normal T-cell effector functions. Exemplary T cell signaling domains are provided herein, and are known in the art. In some aspects an entire intracellular T cell signaling domain can be employed in a CAR. In some aspects it may not be necessary to use the entire signaling domain. In some aspects the signaling domain may be synthetically designed to comprise multiple and chimeric signaling domains.

Examples of intracellular T cell signaling domains for use in the CAR include the cytoplasmic sequences of the T cell receptor (TCR) and co-stimulatory molecules that act in concert to initiate signal transduction following antigen receptor engagement, as well as any derivative or variant of these sequences and any synthetic sequence that has the same functional capability.

T cell receptor signaling domains regulate primary activation of the T cell receptor complex either in a stimulatory way, or in an inhibitory way. As described herein, a primary intracellular signaling domain/sequence produces an intracellular signal when an extracellular domain, e.g., an antigen binding domain, to which it is fused binds cognate antigen. In some aspects it may be derived from a primary stimulatory molecule, e.g., it comprises intracellular sequence of a primary stimulatory molecule. It comprises sufficient primary stimulatory molecule sequence to produce an intracellular signal, e.g., when an antigen binding domain to which it is fused binds cognate antigen. The disclosed CARs can include primary cytoplasmic signaling sequences that act in a stimulatory manner, which may contain signaling motifs that are known as immunoreceptor tyrosine-based activation motifs or ITAMs. In some aspects the signaling domain comprises a modified ITAM domain, e.g., a mutated ITAM domain which has altered (e.g., increased or decreased) activity as compared to the native ITAM domain, a chimeric ITAM or partial ITAM. Examples of ITAM containing primary cytoplasmic signaling sequences that can be included in a disclosed CAR include those from TCR zeta (CD3 zeta), FcRγ (FCER1G), FcεRIβ (MS4A2), CD3 gamma, CD3 delta, CD3 epsilon, CD5, CD22, CD79a, CD79b, FcεRI, DAP10, DAP12, CEACAM4 and CEACAM3 proteins. In several aspects, the cytoplasmic signaling molecule in the CAR includes an intracellular T cell signaling domain from CD3 zeta.

By way of non-limiting example, the intracellular signaling domain of a CAR comprises an immunoreceptor tyrosine-based inhibition motif or ITIM. A signaling domain comprising an ITIM can comprise a conserved sequence of amino acids (S/I/V/LxYxxI/V/L) that is found in the cytoplasmic tails of some inhibitory receptors of the immune system. A primary CAR signaling domain comprising an ITIM can be modified, for example phosphorylated, by enzymes such as a Src kinase family member (e.g., Lck). Following phosphorylation, other proteins, including enzymes, can be recruited to the ITIM. These other proteins include, but are not limited to, enzymes such as the phosphotyrosine phosphatases SHP-1 and SHP-2, the inositol-phosphatase called SHIP, and proteins having one or more SH2 domains (e.g., ZAP70). A CAR intracellular signaling domain can comprise a signaling domain (e.g., ITIM) of BTLA, CD5, CD31, CD66a, CD72, CMRF35H, DCIR, EPO-R, FcγRIIB (CD32), Fc receptor-like protein 2 (FCRL2), Fc receptor-like protein 3 (FCRL3), Fc receptor-like protein 4 (FCRL4), Fc receptor-like protein 5 (FCRL5), Fc receptor-like protein 6 (FCRL6), protein G6b (G6B), interleukin 4 receptor (IL4R), immunoglobulin superfamily receptor translocation-associated 1 (IRTA1), immunoglobulin superfamily receptor translocation-associated 2 (IRTA2), killer cell immunoglobulin-like receptor 2DL1 (KIR2DL1), killer cell immunoglobulin-like receptor 2DL2 (KIR2DL2), killer cell immunoglobulin-like receptor 2DL3 (KIR2DL3), killer cell immunoglobulin-like receptor 2DL4 (KIR2DL4), killer cell immunoglobulin-like receptor 2DL5 (KIR2DL5), killer cell immunoglobulin-like receptor 3DL1 (KIR3DL1), killer cell immunoglobulin-like receptor 3DL2 (KIR3DL2), leukocyte immunoglobulin-like receptor subfamily B member 1 (LIR1), leukocyte immunoglobulin-like receptor subfamily B member 2 (LIR2), leukocyte immunoglobulin-like receptor subfamily B member 3 (LIR3), leukocyte immunoglobulin-like receptor subfamily B member 5 (LIR5), leukocyte immunoglobulin-like receptor subfamily B member 8 (LIR8), leukocyte-associated immunoglobulin-like receptor 1 (LAIR-1), mast cell function-associated antigen (MAFA), NTB-A, programmed cell death protein 1 (PD-1), PILR, SIGLECL1, sialic acid binding Ig like lectin 2 (SIGLEC2 or CD22), sialic acid binding Ig like lectin 3 (SIGLEC3 or CD33), sialic acid binding Ig like lectin 5 (SIGLEC5 or CD170), sialic acid binding Ig like lectin 6 (SIGLEC6), sialic acid binding Ig like lectin 7 (SIGLEC7), sialic acid binding Ig like lectin 10 (SIGLEC10), sialic acid binding Ig like lectin 11 (SIGLEC11), sialic acid binding Ig like lectin 4 (SIGLEC4), sialic acid binding Ig like lectin 8 (SIGLEC8), sialic acid binding Ig like lectin 9 (SIGLEC9), platelet and endothelial cell adhesion molecule 1 (PECAM-1), signal regulatory protein (SIRP 2), and signaling threshold regulating transmembrane adaptor 1 (SIT). In some aspects, the CAR intracellular signaling domain comprises a modified ITIM domain, e.g., a mutated, truncated, and/or optimized ITIM domain, which has altered (e.g., increased or decreased) activity compared to the native ITIM domain.

In some aspects, the CAR intracellular signaling domain comprises at least 2 ITAM domains (e.g., at least 3, 4, 5, 6, 7, 8, 9, or 10 ITAM domains). In some aspects, the CAR intracellular signaling domain comprises at least 2 ITIM domains (e.g., at least 3, 4, 5, 6, 7, 8, 9, or 10 ITIM domains) (e.g., at least 2 primary signaling domains). In some aspects, the CAR intracellular signaling domain comprises both ITAM and ITIM domains. In an aspect, an intracellular signaling domain of subject CAR is from an Fcγ receptor (FcγR), an Fcε receptor (FcεR), an Fcα receptor (FcαR), neonatal Fc receptor (FcRn), CD3, CD37, CD3γ, CD30, CD38, CD4, CD5, CD8, CD21, CD22, CD28, CD32, CD40L (CD154), CD45, CD66d, CD79a, CD79b, CD80, CD86, CD278 (also known as ICOS), CD247 7, CD247 n, DAP10, DAP12, FYN, LAT, Lck, MAPK, MHC complex, NFAT, NF-κB, PLC-γ, iC3b, C3dg, C3d, and Zap70. In another aspect, the intracellular signaling domain of a subject CAR is from CD3, CD3ƒ, CD3γ, CD3δ, and/or CD3ε. In another aspect, the intracellular signaling domain of a subject CAR is from CD3ƒ.

In some aspects the intracellular region of the CAR can include the ITAM containing primary cytoplasmic signaling domain (such as CD3-zeta) by itself or combined with any other desired cytoplasmic domain(s) useful in the context of a CAR. For example, the cytoplasmic domain of the CAR can include primary cytoplasmic signaling domain and an intracellular costimulatory signaling domain. As used herein, a costimulatory signaling domain produces an intracellular signal when an extracellular domain, e.g., an antigen binding domain to which it is fused, or coupled by a dimerization switch, binds cognate ligand. In some aspects the co-stimulatory domain may be derived from, be a functional fragment of, analog of or modified from a costimulatory molecule. It can comprise the entire intracellular region or a fragment of the intracellular region of a costimulatory molecule which is sufficient for generation of an intracellular signal, e.g., when an antigen binding domain to which it is fused, or coupled by a dimerization switch, binds cognate antigen. Non-limiting examples of co-stimulatory domains (identified by the costimulatory molecules from which they are derived) include CD27, MHC class I molecule, SLAMF7, ITGA4, CD11c, CD28, TNF receptor protein, IA4, ITGB1, 4-1BB (CD137), Immunoglobulin-like protein, PD-1, CD49D, CD29, OX40 (CD134), Cytokine receptor, ITGA6, ITGB2, CD30, Integrin, VLA-6, CD18, CD40, Signaling lymphocytic activation molecule (SLAM protein), CD19, CD49f, ITGB7, ICOS (CD278), CD4, ITGAD, ICAM-1, Toll ligand receptor, CD8 alpha, CD11d, TNFR2, LFA-1 (CD11a/, BTLA, CD8 beta ITGAE, TRANCE, CD18, RANKL, CD2, CDS, IL2R beta, CD103, DNAM1 (CD226), CD7 ICAM-1, IL2R gamma, ITGAL, SLAMF4 (CD244, 2B4), LIGHT GITR, IL7R, alpha, CD11a, CD84, BAFFR, ITGA4 ITGAM, CD96 (Tactile), B7-H3, HVEM (LIGHTR), VLA1, CD11b, CEACAM1, a, ligand, that, specifically, binds, with CD83, KIRDS2, CD49a, ITGAX, CRTAM, Ly9, (CD229), CD160, (BY55), PSGL1, CD100, (SEMA4D), CD69, SLAMF6, (NTB-A, Ly108), SLAM, (SLAMF1, CD150, IPO-3), BLAME (SLAMF8), SELPLG (CD162), LTBR, LAT, GADS, SLP-76, PAG/Cbp, CD19a, or other cytoplasmic regions capable of transmitting a co-stimulatory signal as a result of interacting with other proteins that bind to a ligand. An additional example of a signaling domain that can be included in a disclosed CARs is a Tumor necrosis factor receptor superfamily member 18 (TNFRSF18; also known as glucocorticoid-induced TNFR-related protein, GITR) signaling domain.

In some aspects the costimulatory signaling domain, has at least 70, 75, 80, 85, 90, 95, 96, 97, 98, or 99% similarity or identity with the corresponding residues of a naturally occurring stimulatory molecule.

The intracellular signaling sequences within the cytoplasmic domain may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling sequences. In some aspects, a glycine-serine doublet can be used as a suitable linker. In some aspects, a single amino acid, e.g., an alanine, a glycine, can be used as a suitable linker.

In some aspects, the intracellular signaling domain is designed to comprise two or more, e.g., 2, 3, 4, 5, or more, costimulatory signaling domains. In some aspects, the two or more, e.g., 2, 3, 4, 5, or more, costimulatory signaling domains, are separated by a linker molecule, e.g., a linker molecule described herein. As described herein, the intracellular signaling domain comprises two costimulatory signaling domains. As described herein, the linker molecule is a glycine residue. As described herein, the linker is an alanine residue. Further examples of linkers are provided.

In some exemplary aspects, the CAR includes a CD3 zeta signaling domain (either natural SEQ ID NOS 4 or mutant (Q/K) for example SEQ ID. NO 5), a CD28 signaling domain (SEQ ID: 6): a CD137 (4-1BB) (SEQ ID 7) signaling domain, derivatives or fragments thereof or a combination of two or more thereof. In some aspects, the cytoplasmic domain includes the signaling domain of CD3-zeta and the signaling domain of CD28. In some aspects, the cytoplasmic domain includes the signaling domain of CD3 zeta and the signaling domain of CD137. In some aspects, the cytoplasmic domain includes the signaling domain of CD3-zeta and the signaling domain of CD28 and CD137. The order of the one or more T cell signaling domains on the CAR can be varied as needed by the person of ordinary skill in the art.

In some exemplary aspects the CAR can include the signaling domains listed in Table D. In some aspects of the current disclosure the signaling domain comprises a sequences that has at least 70, 75, 80, 85, 90, 95, 96, 97, 98, or 99% similarity or identity to SEQ ID NOS: 8-11.

TABLE D
Intracellular region sequences

| SEQ | | |
|---|---|---|
| ID | Name (signaling | |
| NO | domain from source) | SEQUENCE |
| 8 | CD3z | RVKFSRSADAPAYQQGQNQLYNELNLGRREEYDVLDKRRGRDPEMGGKPRRKNP |
| | | QEGLYNELQKDKMAEAYSEIGMKGERRRGKGHDGLYQGLSTATKDTYDALHMQAL |
| | | PPR |
| 9 | CD3z(Q/K) mutant | RVKFSRSADAPAYKQGQNQLYNELNLGRREEYDVLDKRRGRDPEMGGKPRRKNP |
| | | QEGLYNELQKDKMAEAYSEIGMKGERRRGKGHDGLYQGLSTATKDTYDALHMQAL |
| | | PPR |
| 10 | CD28 | SKRSRLLHSDYMNMTPRRPGPTRKHYQPYAPPRDFAAYRS |
| 11 | CD137 (4-1BB) | KRGRKKLLYIFKQPFMRPVQTTQEEDGCSCRFPEEEEGGCEL |

In some aspects, one cytoplasmic domain is linked to a second cytoplasmic domain. In other aspects, one cytoplasmic domain is linked to two or more other cytoplasmic domains. The cytoplasmic domains can be the same or different. For example, the cytoplasmic domain of a co-stimulatory molecule can be linked to the cytoplasmic domain of one or more of the CD3 chains of the T cell receptor, for example to one or more of the zeta, eta, delta, gamma or epsilon CD3 chains of the T cell receptor. In other aspects, the cytoplasmic domain of a co-stimulatory molecule a tyrosine kinase, such as a member of the Syk tyrosine kinase family which activates cytolysis, Syk or ZAP-70, where the cytoplasmic domain is capable of activating effector function in a host cell.

In a non-limiting example, the C-terminus of a CD28 receptor is joined to the N-terminal residue of the cytoplasmic domain of CD3 zeta (i.e., linked head-to-tail), resulting in a CAR with targeting (extracellular) and transmembrane segments linked to the cytoplasmic domains of CD3-zeta and CD28. In another specific, non-limiting example of a CAR, a CD4 extracellular domain (CD4 targeting segment) is linked to the cytoplasmic domains of CD3-zeta and CD28 via a transmembrane domain. In yet another specific, non-limiting example of a CAR, a targeting (extracellular) domain containing two targeting domains (CD4 and scFv) is linked to the cytoplasmic domains of CD3-zeta and CD28 via a transmembrane domain. In a further specific, non-limiting example of a CAR, an extracellular domain containing two targeting domains (CD4 and scFv17b) is linked to the cytoplasmic domains of CD3-zeta and CD28 via a transmembrane domain. In some aspects of the CARs disclosed herein, the cytoplasmic signaling domain is a combined cytoplasmic domain comprising an effector function signaling domain, e.g. zeta, linked to a co-stimulatory signaling domain such as CD28. Thus, binding of the appropriate ligand, e.g. gp120, to an extracellular domain (for example, CD4 or scFv) results in the transduction of both a primary activation signal and a co-stimulatory signal simultaneously, in an MHC-independent manner.

In some aspects, the CAR may further comprise hinge or linkers between various domains. Non-limiting examples of such linkers are provided in Table E. These linkers may be located at any region deemed functional CAR.

TABLE E
Linker sequences

| SEQ | | |
|---|---|---|
| ID | | |
| NO: | Description | Sequence |
| 12 | Flexible linker | TSGS |
| 13 | Flexible linker | GGGGS |
| 14 | Flexible linker | GGGS |
| N/A | Flexible linker | GG |
| 16 | Flexible linker | KESGSVSSEQLAQFRSLD |
| 17 | Flexible linker | EGKSSGSGSESKST |
| 18 | Flexible linker | GSAGSAAGSGEF |
| 19 | Rigid linker | EAAAK |
| 20 | Rigid linker | EAAAR |
| 21 | Rigid linker | PAPAP |
| 22 | Rigid linker | AEAAAKEAAAKA |
| 23 | Rigid linker | ILTHDSSIRYLQEIYNSNNQKIVNLKEK |
| | | VAQLEAQCQEPCKDTVQIHDITG |
| N/A | Flexible linker | GGS |
| 25 | Flexible linker | SLNGGGGSGGGGSGGGGSGGGGSGGGGS |
| | | TS |
| 26 | Flexible linker | SGGSGGGGSGGGSGGGGSLQ |
| 27 | Flexible linker | SGGGSGGGGSGGGGSGGGGSGGGSLQ |
| 28 | Flexible linker | GGGGSGGGGSGGGGS |

In some exemplary aspects the CAR is a BCMA CAR (i.e., B cell maturation antigen, SEQ ID NO 29). In an aspect, the CAR is a CD19 CAR (SEQ ID NO 30). In an aspect, the CAR is a CEA CAR (SEQ ID NO: 31). In some exemplary aspects of the current disclosure the CAR sequence has at least 70, 75, 80, 85, 90, 95, 96, 97, 98, or 99% similarity or identity to SEQ ID NOS: 29, 30, or 31 as provided in Table F.

TABLE F
CAR sequences

| SEQ | | |
|---|---|---|
| ID | | |
| NO | Name | SEQUENCE |
| 29 | BCMA CAR | MALPVTALLLPLALLLHAARPDIVLTQSPPSLAMSLGKRATISCRASESVTILGSHLIHW |
| | | YQQKPGQPPTLLIQLASNVQTGVPARFSGSGSRTDFTLTIDPVEEDDVAVYYCLQSRT |
| | | IPRTFGGGTKLEIKGSTSGSGKPGSGEGSTKGQIQLVQSGPELKKPGETVKISCKASG |
| | | YTFTDYSINWVKRAPGKGLKWMGWINTETREPAYAYDFRGRFAFSLETSASTAYLQI |
| | | NNLKYEDTATYFCALDYSYAMDYWGQGTSVTVSSTTTPAPRPPTPAPTIASQPLSLR |
| | | PEACRPAAGGAVHTRGLDFACDIYIWAPLAGTCGVLLLSLVITLYCNHRNRSKRSRLL |
| | | HSDYMNMTPRRPGPTRKHYQPYAPPRDFAAYRSRVKFSRSADAPAYQQGQNQLYN |
| | | ELNLGRREEYDVLDKRRGRDPEMGGKPRRKNPQEGLYNELQKDKMAEAYSEIGMK |
| | | GERRRGKGHDGLYQGLSTATKDTYDALHMQALPPR |
| 30 | CD19 CAR | MALPVTALLLPLALLLHAARPDIQMTQTTSSLSASLGDRVTISCRASQDISKYLNWYQQ |
| | | KPDGTVKLLIYHTSRLHSCVPSRFSGSGSGTDYSLTISNLEQEDIATYFCQQGNTLPYT |
| | | FGGGTKLEITGGGGSGGGGSGGGGSEVKLQESGPGLVAPSQSLSVTCTVSGVSLPD |
| | | YGVSWIRQPPRKCLEWLGVIWGSETTYYNSALKSRLTIIKDNSKSQVFLKMNSLQTDD |
| | | TAIYYCAKHYYYGGSYAMDYWGQGTSVTVSSTTTPAPRPPTPAPTIASQPLSLRPEA |
| | | CRPAAGGAVHTRGLDFACDIYIWAPLAGTCGVLLLSLVITLYCKRGRKKLLYIFKQPFM |
| | | RPVQTTQEEDGCSCRFPEEEEGGCELRVKFSRSADAPAYKQGQNQLYNELNLGRR |
| | | EEYDVLDKRRGRDPEMGGKPRRKNPQEGLYNELQKDKMAEAYSEIGMKGERRRGK |
| | | GHDGLYQGLSTATKDTYDALHMQALPPR |
| 31 | CEA CAR | MDMRVPAQLLGLLLLWLRGARCDIQLTQSPSSLSASVGDRVTITCKASQDVGTSVAW |
| | | YQQKPGKAPKLLIYWTSTRHTGVPSRFSGSGSGTDFTFTISSLQPEDIATYYCQQYSL |
| | | YRSFGQGTKVEIKRGGSGSGGSGSGGSGSEVQLVESGGGVVQPGRSLRLSCSASG |
| | | FDFTTYWMSWVRQAPGKGLEWIGEIHPDSSTINYAPSLKDRFTISRDNAKNTLFLQM |
| | | DSLRPEDTGVYFCASLYFGFPWFAYWGQGTPVTVSSAKPTTTPAPRPPTPAPTIASQ |
| | | PLSLRPEAARPAAGGAVHTRGLDFAPRKIEVMYPPPYLDNEKSNGTIIHVKGKHLCPS |
| | | PLFPGPSKPFWVLVVVGGVLACYSLLVTVAFIIFWVRSKRSRLLHSDYMNMTPRRPG |
| | | PTRKHYQPYAPPRDFAAYRSRVKFSRSAEPPAYQQGQNQLYNELNLGRREEYDVLD |
| | | KRRGRDPEMGGKPRRKNPQEGLYNELQKDKMAEAYSEIGMKGERRRGKGHDGLY |
| | | QGLSTATKDTYDALHMQALPPR |

ii. Gamma Delta TCRs

In some aspects the current disclosure encompasses multicistronic polynucleotides comprising nucleic acids encoding monomers of heterodimeric γδT-cell receptor in addition to nucleic acids encoding a CAR. In the context of the disclosure, “gamma”, “γ”, and “g” are used interchangeably to refer to a γ chain or monomer of a γδ TCR. “Delta”, “δ”, and “d” are used interchangeably to refer to a δ chain or monomer of a γδ TCR.

In some aspects the polynucleotide can comprise nucleic acids encoding one or more of (a) a γ-chain selected from the group consisting of γ2, γ3, γ4, γ5, γ8, γ9, and γ11, and derivatives, variations or fragments thereof; (b) a δ-chain selected from the group consisting of δ1, δ2, δ3, and δ5, and derivatives, variations or fragments thereof, or (c) any combination of (a) and (b).

In some preferred aspects, the γ-chain is the γ9 chain and the δ-chain is the δ2 chain. In some other preferred aspects, the γ-chain is the γ4 chain and the δ-chain is the δ5 chain. In some aspects, the γ-chain is the γ2 chain and the δ-chain is the δ1 chain. In some aspects, the γ-chain is the γ3 chain and the δ-chain is the δ1 chain. In some aspects, the γ-chain is the γ4 chain and the δ-chain is the δ1 chain. In some aspects, the γ-chain is the γ5 chain and the δ-chain is the δ1 chain. In some aspects, the γ-chain is the γ8 chain and the δ-chain is the δ1 chain. In some aspects, the γ-chain is the γ9 chain and the δ-chain is the δ1 chain. In some aspects, the γ-chain is the γ11 chain and the δ-chain is the δ1 chain.

In some aspects, the γ-chain is the γ2 chain and the δ-chain is the δ2 chain. In some aspects, the γ-chain is the γ3 chain and the δ-chain is the δ2 chain. In some aspects, the γ-chain is the γ4 chain and the δ-chain is the δ2 chain. In some aspects, the γ-chain is the γ5 chain and the δ-chain is the δ2 chain. In some aspects, the γ-chain is the γ8 chain and the δ-chain is the δ2 chain. In some aspects, the γ-chain is the γ9 chain and the δ-chain is the δ2 chain. In some aspects, the γ-chain is the γ11 chain and the δ-chain is the δ2 chain.

In some aspects, the γ-chain is the γ2 chain and the δ-chain is the δ3 chain. In some aspects, the γ-chain is the γ3 chain and the δ-chain is the δ3 chain. In some aspects, the γ-chain is the γ4 chain and the δ-chain is the δ3 chain. In some aspects, the γ-chain is the γ5 chain and the δ-chain is the δ3 chain. In some aspects, the γ-chain is the γ8 chain and the δ-chain is the δ3 chain. In some aspects, the γ-chain is the γ9 chain and the 0-chain is the δ3 chain. In some aspects, the γ-chain is the γ11 chain and the δ-chain is the δ3 chain.

In some aspects, the γ-chain is the γ2 chain and the δ-chain is the δ5 chain. In some aspects, the γ-chain is the γ3 chain and the δ-chain is the δ5 chain. In some aspects, the γ-chain is the γ4 chain and the δ-chain is the δ5 chain. In some aspects, the γ-chain is the γ5 chain and the δ-chain is the δ5 chain. In some aspects, the γ-chain is the γ8 chain and the δ-chain is the δ5 chain. In some aspects, the γ-chain is the γ9 chain and the δ-chain is the δ5 chain. In some aspects, the γ-chain is the γ11 chain and the δ-chain is the δ5 chain.

In some aspects, the polynucleotide comprises nucleic acids encoding a variable domain from a Y-chain and/or a variable domain from a δ-chain. Variable domains can be indicated by a V preceding the γ-chain and δ-chain designations, e.g., Vγ2, Vγ3, Vγ4, Vγ5, Vγ8, Vγ9, Vγ11, Vδ1, Vδ2, Vδ3, and Vδ5.

In some aspects, the polynucleotide comprises nucleic acids encoding (a) a variable domain of a γ-chain selected from the group consisting of Vγ2, Vγ3, Vγ4, Vγ5, Vγ8, Vγ9, and Vγ11; (b) a variable domain of a δ-chain selected from the group consisting of Vδ1, Vδ2, Vδ3, and Vδ5; or (c) any combination of (a) and (b), e.g., as indicated herein for the γ and δ chains. In some aspects, the γ-chain variable domain is the Vγ9 and the δ-chain variable domain is the Vδ2. In some aspects, the γ-chain variable domain is the Vγ4 and the δ-chain variable domain is the Vδ5.

In some aspects, the polynucleotide comprises nucleic acids encoding a constant domain from a γ-chain and/or a constant domain from a δ-chain. Constant domains can be indicated by a C preceding the γ-chain and δ-chain designations, e.g., Cγ1, Cγ2 and Co.

In some aspects, where the polynucleotide comprises nucleic acids encoding (a) a constant domain of a γ-chain selected from the group consisting of Cγ1 and Cγ2; (b) a constant domain of a δ-chain Co; or (c) any combination of (a) and (b), e.g., as indicated herein for the γ and δ chains. In some aspects, the γ-chain constant domain is the Cγ1 and the δ-chain constant domain is the Co. In some aspects, the γ-chain constant domain is the Cγ2 and the δ-chain constant domain is the Co.

In some aspects, the polynucleotide may comprise nucleic acids encoding a Vγ9V2 TCR or functional fragment thereof. The Vγ9Vδ2 TCR can comprise at least one of a γ-TCR amino acid sequence or a δ-TCR amino acid sequence capable of recognizing a CD277 protein on a cell surface of a cell (e.g. tumor cell). In some aspects, the encoded receptor comprises a variant or a fragment of at least one of a γ-TCR amino acid sequence or a δ-TCR amino acid sequence capable of recognizing a CD277 protein on a cell surface of a target cell. The present disclosure contemplates receptors comprising any portion or fragment or variation of a γδTCR capable of recognizing a cell (e.g. tumor cell) via a CD277 cell surface molecule. Variable domain and CDR regions (CDR1, CDR2, and CDR3) for two such γδTCRs are identified in Table G: SEQ ID NO: 34, 37, 40-41, 51-52, 61-62 and 166-172, and are described further above.

In some aspects the polynucleotide may comprise nucleic acids encoding a variant or a fragment of at least one of a γ-TCR amino acid sequence and/or a δ-TCR amino acid sequence capable of recognizing an endothelial protein c receptor (EPCR) protein on a cell surface of a target cell. The present disclosure contemplates nucleic acids encoding a polynucleotide comprising nucleic acids encoding any portion or fragment or variation of a γδTCR capable of recognizing a cell (e.g., tumor cell) via an EPCR cell surface molecule. Variable domain and CDR3 regions for such a γδTCR are identified in Table G: SEQ ID NO: 51-52.

In some aspects, the polynucleotide may comprise nucleic acids encoding a variant or a fragment of at least one of a γ-TCR amino acid sequence and/or a δ-TCR amino acid sequence capable of recognizing annexin A2 on a cell surface of a target cell. The present disclosure contemplates nucleic acids encoding a polynucleotide comprising nucleic acids encoding any portion or fragment or variation of a γδTCR capable of recognizing a cell (e.g., tumor cell) via an annexin A2 surface molecule. Variable domain and CDR3 regions for such a γδTCR are identified in Table G: SEQ ID NO: 80 and 81.

In some aspects, the polynucleotide comprises nucleic acids encoding a variant or a fragment of at least one of a γ-TCR amino acid sequence and/or a δ-TCR amino acid sequence capable of recognizing aberrant HLA protein expression on a cell surface of a target cell. The present disclosure contemplates a polynucleotide comprising nucleic acids encoding any portion or fragment or variation of a γδTCR capable of recognizing a cell (e.g. tumor cell) via an aberrant HLA protein expression on the cell surface. In some aspects, the polynucleotide comprises nucleic acids encoding a variant or a fragment of at least one of a γ-TCR amino acid sequence and/or a δ-TCR amino acid sequence capable of recognizing cancers in an MHC-unrestricted manner. Variable domain and CDR3 regions for such a γδTCR are identified in Table G: SEQ ID NO: 32 and 35.

In some aspects, the polynucleotide comprises nucleic acids encoding at least a portion of a Cγ or Cδ region and at least a portion of a Vγ or a Vδ region of a γδTCR. In some aspects, the polynucleotide comprises nucleic acids encoding at least a portion of a Cγ or Cδ region and at least a CDR3 domain of either a Vγ or a Vδ domain of a γδTCR. In some aspects, the polynucleotide comprises nucleic acids encoding all CDR regions of the Vγ9Vδ2 TCR, and all of the CDR regions can be involved in binding to a cell surface molecule (e.g. CD277 molecule) on the surface of a cell In some aspects, the polynucleotide comprises nucleic acids encoding all CDR regions of the Vγ4Vδ5 TCR, and all of the CDR regions can be involved in binding to a cell surface molecule (e.g. EPCR molecule) on the surface of a cell. In some aspects, the exogenous antigen-recognition receptor comprises all CDR regions of the Vγ5Vδ1 TCR, and all of the CDR regions can be involved in binding to a cell surface molecule (e.g. HLA molecule) on the surface of a cell. In some aspects, the polynucleotide comprises nucleic acids encoding CDR regions of the Vγ8V53 TCR, and all of the CDR regions can be involved in binding to a cell surface molecule (e.g. annexin A2) on the surface of a cell.

Gamma-delta TCRs useful in compositions and methods of the disclosure, and sequences thereof, have been disclosed for example, in patent applications WO2013147606A1, WO2017212074A1, and WO2018211115A1, each of which is incorporated herein by reference in its entirety. These sequences are identified in Table G.

Non-limiting examples of nucleic acid sequences of a γδ TCR comprises a sequence that codes a γ-chain (G), δ-chain (D), a variable domain (TRG, TRD), a CDR (e.g., CDR3) sequence therefrom, a constant domain (TRDC, TRGC1, TRGC2), or a combination thereof selected from Table G. An example of a suitable TRDC is represented by SEQ ID NO: 100, an example of a suitable TRGC1 is represented by SEQ ID NO: 101 and an example of a suitable TRGC2 is represented by SEQ ID NO: 102. Example of a sequence is published (Gründer C., et al, Blood 2012; 120 (26): 5153-5162. Doi: https://doi.org/10.1182/blood-2012-05-432427). In some cases, a polynucleotide comprises nucleic acids encoding a sequence (e.g., a CDR3 region sequence) with at least 60%, at least 65%, at least 70%, at least 75%, at least 80%, at least 85%, at least 90%, at least 95%, at least 97%, at least 98%, at least 99%, or up to 100% sequence similarity or identity to a sequence in Table G. In some cases, a polynucleotide comprises nucleic acids encoding a sequence (e.g., a CDR1, CDR2, CDR3 or variable domain) with at least 60%, at least 65%, at least 70%, at least 75%, at least 80%, at least 85%, at least 90%, at least 95%, at least 97%, at least 98%, at least 99%, or up to 100% sequence similarity or identity to one or more of SEQ ID NOs: 34, 37, 40, 41, 51, 52, 166, 167, 168, 169, 170, 171, and 172, as described above.

TABLE G
Sequences for elements and types of yð TCR

| SEQ ID | | |
|---|---|---|
| NO: | Name | Sequence |
| 32 | CDR3 VD1 Fe11 | CALGDSYGGGPLYTDKLIF |
| 33 | CDR3 VD2 cl3 | CACDLLGYTDKLIF |
| 34 | CDR3 VD2 cl5 | CACDALKRTDTDKLIFG |
| 35 | CDR3 VG5 Fe11 | CATWDRPEIYYKKLF |
| 36 | CDR3 VG9 cl3 | CALWEEELGKKIKVF |
| 37 | CDR3 VG9 cl5 | CALWEIQELGKKIKVF |
| 38 | TRD AB3 | MERISSLIHLSLFWAGVMSAIELVPEHQTVPVSIGVPATLRCSMKGEAIGNY |
| | | YINWYRKTQGNTMTFIYREKDIYGPGFKDNFQGDIDIAKNLAVLKILAPSER |
| | | DEGSYYCACDLLGYTDKLIFGKGTRVTVEPR |
| 39 | TRG AB4 | MVSLLHASTLAVLGALCVYGAGHLEQPQISSTKTLSKTARLECVVSGITISA |
| | | TSVYWYRERPGEVIQFLVSISYDGTVRKESGIPSGKFEVDRIPETSTSTLTIH |
| | | NVEKQDIATYYCALWEEELGKKIKVFGPGTKLIIT |
| 40 | TRD cl5 | MERISSLIHLSLFWAGVMSAIELVPEHQTVPVSIGVPATLRCSMKGEAIGNY |
| | | YINWYRKTQGNTMTFIYREKDIYGPGFKDNFQGDIDIAKNLAVLKILAPSER |
| | | DEGSYYCACDALKRTDTDKLIFGKGTRVTVEPR |
| 41 | TRG cl5 | MVSLLHASTLAVLGALCVYGAGHLEQPQISSTKTLSKTARLECVVSGITISA |
| | | TSVYWYRERPGEVIQFLVSISYDGTVRKESGIPSGKFEVDRIPETSTSTLTIH |
| | | NVEKQDIATYYCALWEIQELGKKIKVFGPGTKLIIT |
| 42 | TRD Fe11 | MVFSSLLCVFVAFSYSGSSVAQKVTQAQSSVSMPVRKAVTLNCLYETSW |
| | | WSYYIFWYKQLPSKEMIFLIRQGSDEQNAKSGRYSVNFKKAAKSVALTISA |
| | | LQLEDSAKYFCALGDSYGGGPLYTDKLIFGKGTRVTVEPR |
| 43 | TRG Fe11 | MGWALLVLLAFLSPASQKSSNLEGGTKSVTRPTRSSAEITCDLTVINAFYIH |
| | | WYLHQEGKAPQRLLYYDVSNSKDVLESGLSPGKYYTHTPRRWSWILILRN |
| | | LIENDSGVYYCATWDRPEIYYKKLFGSGTTLVVT |
| 44 | CDR3 VG4 E113 | CATWDGPPYYKKLF |
| 45 | CDR3 VG2 F4 | CATWDGQKKLF |
| 46 | CDR3 VG8 Zi11 | CATWDNYKKLF |
| 47 | CDR3 VD5 D37 | CAASSPIRGYTGSDKLIF |
| 48 | CDR3 VD5 E113 | CAASSPIRGYTGSDKLIF |
| 49 | CDR3 VD1 F4 | CALGELRYWGIVDKLIF |
| 50 | CDR3 VD1 Zi11 | CALGELRGQISFLYLLGDTTDKLIF |
| 51 | CDR3 VG4 D25 | CATWDGFYYKKLF |
| 52 | CDR3 VD5 D25 | CAASSPIRGYTGSDKLIF |
| 53 | TRD E113 | MAMLLGASVLILWLQPDWVNSQQKNDDQQVKQNSPSLSVQEGRISILNCD |
| | | YTNSMFDYFLWYKKYPAEGPTFLISISSIKDKNEDGRFTVFLNKSAKHLSLHI |
| | | VPSQPGDSAVYFCAASSPIRGYTGSDKLIFGKGTRVTVEPR |
| 54 | TRG E113 | MEWALAVLLAFLSPASQKSSNLEGRTKSVIRQTGSSAEITCDLAEGSTGYI |
| | | HWYLHQEGKAPQRLLYYDSYTSSVVLESGISPGKYDTYGSTRKNLRMILR |
| | | NLIENDSGVYYCATWDGPPYYKKLFGSGTTLVVT |
| 55 | TRD F4 | MVFSSLLCVFVAFSYSGSSVAQKVTQAQSSVSMPVRKAVTLNCLYETSW |
| | | WSYYIFWYKQLPSKEMIFLIRQGSDEQNAKSGRYSVNFKKAAKSVALTISA |
| | | LQLEDSAKYFCALGELRYWGIVDKLIFGKGTRVTVEPR |
| 56 | TRG F4 | MEWALAVLLAFLSPASQKSSNLEGRTKSVIRQTGSSAEITCDLAEGSNGYI |
| | | HWYLHQEGKAPQRLQYYDSYNSKVVLESGVSPGKYYTYASTRNNLRLILR |
| | | NLIENDSGVYYCATWDGQKKLFGSGTTLVVT |
| 57 | TRD Zi11 | MVFSSLLCVFVAFSYSGSSVAQKVTQAQSSVSMPVRKAVTLNCLYETSW |
| | | WSYYIFWYKQLPSKEMIFLIRQGSDEQNAKSGRYSVNFKKAAKSVALTISA |
| | | LQLEDSAKYFCALGELRGQISFLYLLGDTTDKLIFGKGTRVTVEPR |
| 58 | TRG Zi11 | MVLALALLLAFLPPASQKSSNLEGRTKSVTRPTGSSAVITCDLPVENAVYTH |
| | | WYLHQEGKAPQRLLYYDSYNSRVVLESGISREKYHTYASTGKSLKFILENLI |
| | | ERDSGVYYCATWDNYKKLFGSGTTLVVT |
| 59 | TRD D37 | MAMLLGASVLILWLQPDWVNSQQKNDDQQVKQNSPSLSVQEGRISILNCD |
| | | YTNSMFDYFLWYKKYPAEGPTFLISISSIKDKNEDGRFTVFLNKSAKHLSLHI |
| | | VPSQPGDSAVYFCAASSPIRGYTGSDKLIFGKGTRVTVEPR |
| 60 | TRG D37 | MVLALALLLAFLPPASQKSSNLEGRTKSVTRPTGSSAVITCDLPVENAVYTH |
| | | WYLHQEGKAPQRLLYYDSYNSRVVLESGISREKYHTYASTGKSLKFILENLI |
| | | ERDSGVYYCATWDNYMKLFGSGTTLVVT |
| 61 | TRD D25 | MAMLLGASVLILWLQPDWVNSQQKNDDQQVKQNSPSLSVQEGRISILNCD |
| | | YTNSMFDYFLWYKKYPAEGPTFLISISSIKDKNEDGRFTVFLNKSAKHLSLHI |
| | | VPSQPGDSAVYFCAASSPIRGYTGSDKLIFGKGTRVTVEPR |
| 62 | TRG D25 | MEWALAVLLAFLSPASQKSSNLEGRTKSVIRQTGSSAEITCDLAEGSTGYI |
| | | HWYLHQEGKAPQRLLYYDSYTSSVVLESGISPGKYDTYGSTRKNLRMILR |
| | | NLIENDSGVYYCATWDGFYYKKLFGSGTTLVVT |
| 63 | CDR3 VG8 D37 | CATWDNYMKLF |
| 64 | CDR3 VD3 F2 | CASSYTLKLGDTPGRVRDWKLIF |
| 65 | CDR3 VG4 F2 | CATWDGPPYYKKLF |
| 66 | CDR3 VD1 Ze11 | CALGDYLGDKYPSYDLLGDTTDKLIF |
| 67 | CDR3 VG8 Ze11 | CATWDNYKKLF |
| 68 | CDR3 VD5 B23 | CAASSPIRGYTGSDKLIF |
| 69 | CDR3 VG8 B23 | CATWDNYKKLF |
| 70 | TRD F2 | MILTVGFSFLFFYRGTLCDKVTQSSPDQTVASGSEVVLLCTYDTVYSNPDL |
| | | FWYRIRPDYSFQFVFYGDNSRSEGADFTQGRFSVKHILTQKAFHLVISPVR |
| | | TEDSATYYCASSYTLKLGDTPGRVRDWKLIFGKGTRVTVEPR |
| 71 | TRG F2 | MEWALAVLLAFLSPASQKSSNLEGRTKSVIRQTGSSAEITCDLAEGSTGYI |
| | | HWYLHQEGKAPQRLLYYDSYTSSVVLESGISPGKYDTYGSTRKNLRMILR |
| | | NLIENDSGVYYCATWDGPPYYKKLFGSGTTLVVT |
| 72 | TRD Ze11 | MVFSSLLCVFVAFSYSGSSVAQKVTQAQSSVSMPVRKAVTLNCLYETSW |
| | | WSYYIFWYKQLPSKEMIFLIRQGSDEQNAKSGRYSVNFKKAAKSVALTISA |
| | | LQLEDSAKYFCALGDYLGDKYPSYDLLGDTTDKLIFGKGTRVTVEPR |
| 73 | TRG Ze11 | MVLALALLLAFLPPASQKSSNLEGRTKSVTRPTGSSAVITCDLPVENAVYTH |
| | | WYLHQEGKAPQRLLYYDSYNSRVVLESGISREKYHTYASTGKSLKFILENLI |
| | | ERDSGVYYCATWDNYKKLFGSGTTLVVT |
| 74 | TRD B23 | MAMLLGASVLILWLQPDWVNSQQKNDDQQVKQNSPSLSVQEGRISILNCD |
| | | YTNSMFDYFLWYKKYPAEGPTFLISISSIKDKNEDGRFTVFLNKSAKHLSLHI |
| | | VPSQPGDSAVYFCAASSPIRGYTGSDKLIFGKGTRVTVEPR |
| 75 | TRG B23 | MVLALALLLAFLPPASQKSSNLEGRTKSVTRPTGSSAVITCDLPVENAVYTH |
| | | WYLHQEGKAPQRLLYYDSYNSRVVLESGISREKYHTYASTGKSLKFILENLI |
| | | ERDSGVYYCATWDNYKKLFGSGTTLVVT |
| 76 | CDR3 VD1 B9 | CALGNGNHIGYWRYTDKLIF |
| 77 | CDR3 VG5 B9 | CATWDRLYYKKLF |
| 78 | TRD B9 | MVFSSLLCVFVAFSYSGSSVAQKVTQAQSSVSMPVRKAVTLNCLYETSW |
| | | WSYYIFWYKQLPSKEMIFLIRQGSDEQNAKSGRYSVNFKKAAKSVALTISA |
| | | LQLEDSAKYFCALGNGNHIGYWRYTDKLIFGKGTRVTVEPR |
| 79 | TRG B9 | MVWALLVLLAFLSPASQKSSNLEGGTKSVTRPTRSSAEITCDLTVINAFYIH |
| | | WYLHQEGKAPQRLLYYDVSNSKDVLESGLSPGKYYTHTPRRWSWILILRN |
| | | LIENDSGVYYCATWDRLYYKKLFGSGTTLVVT |
| 80 | CDR3 VG8 An2 | CATWDSSKLF |
| 81 | CDR3 VD3 An2 | CAFTGLGDTSHADKLIF |
| 82 | TRG An2 | MLLALALLLAFLPPASQKSSNLEGRTKSVTRPTGSSAVITCDLPVENAVYTH |
| | | WYLHQEGKAPQRLLYYDSYNSRVVLESGISREKYHTYASTGKSLKFILENLI |
| | | ERDSGVYYCATWDSSKLFGSGTTLVVT |
| 83 | TRD An2 | MILTVGFSFLFFYRGTLCDKVTQSSPDQTVASGSEVVLLCTYDTVYSNPDL |
| | | FWYRIRPDYSFQFVFYGDNSRSEGADFTQGRFSVKHILTQKAFHLVISPVR |
| | | TEDSATYYCAFTGLGDTSHADKLIFGKGTRVTVEPR |
| 84 | TRD S1 | MILTVGFSFLFFYRGTLCDKVTQSSPDQTVASGSEVVLLCTYDTVYSNPDL |
| | | FWYRIRPDYSFQFVFYGDNSRSEGADFTQGRFSVKHILTQKAFHLVISPVR |
| | | TEDSATYYCAFIVYDFLGELGEDTDKLIFGKGTRVTVEPR |
| 85 | TRD S1 CDR3 | CAFIVYDFLGELGEDTDKLIF |
| 86 | TRG S1 CDR3 | CATWDSKPHYYKKLF |
| 87 | TRG S1 | MRWALLVLLAFLSPASQKSSNLEGRTKSVTRQTGSSAEITCDLTVTNTFYIH |
| | | WYLHQEGKAPQRLLYYDVSTARDVLESGLSPGKYYTHTPRRWSWILRLQ |
| | | NLIENDSGVYYCATWDSKPHYYKKLFGSGTTLVVTDKQLDADVSPKPTIFL |
| | | PSIAETKLQKAGTYLCLLEKFFPDIIKIHWQEKKSNTILGSQEGNTMKINDTY |
| | | MKFSWLTVPEESLDKEHRCIVRHENNKNGIDQEIIFPPIKTDVTTV |
| 88 | TRD S2 CDR3 | CACGLPRSLADKLIF |
| 89 | TRD S2 | MILTVGFSFLFFYRGTLCDKVTQSSPDQTVASGSEVVLLCTYDTVYSNPDL |
| | | FWYRIRPDYSFQFVFYGDNSRSEGADFTQGRFSVKHILTQKAFHLVISPVR |
| | | TEDSATYYCACGLPRSLADKLIFGKGTRVTVEPR |
| 90 | TRG S2 CDR3 | CATWDLHKKLF |
| 91 | TRG S2 | MEWALAVLLAFLSPASQKSSNLEGRTKSVIRQTGSSAEITCDLAEGSTGYI |
| | | HWYLHQEGKAPQRLLYYDSYTSSVVLESGISPGKYDTYGSTRKNLRMILR |
| | | NLIENDSGVYYCATWDLHKKLFGSGTTLVVTDKQLDADVSPKPTIFLPSIAE |
| | | TKLQKAGTYLCLLEKFFPDIIKIHWQEKKSNTILGSQEGNTMKTNDTYMKFS |
| | | WLTVPEESLDKEHRCIVRHENNKNGIDQEIIFPPIKTDVTTV |
| 92 | TRD S3 CDR3 | CALGEFPYWGIPDKLIF |
| 93 | TRD S3 | MVFSSLLCVFVAFSYSGSSVAQKVTQAQSSVSMPVRKAVTLNCLYETSW |
| | | WSYYIFWYKQLPSKEMIFLIRQGSDEQNAKSGRYSVNFKKAAKSVALTISA |
| | | LQLEDSAKYFCALGEFPYWGIPDKLIFGKGTRVTVEPR |
| 94 | TRG S3 CDR3 | CATWDGYYKKLF |
| 95 | TRG S3 | MRWALLVLLAFLSPASQKSSNLEGRTKSVTRQTGSSAEITCDLTVTNTFYIH |
| | | WYLHQEGKAPQRLLYYDVSTARDVLESGLSPGKYYTHTPRRWSWILRLQ |
| | | NLIENDSGVYYCATWDGYYKKLFGSGTTLVVTDKQLDADVSPKPTIFLPSIA |
| | | ETKLQKAGTYLCLLEKFFPDVIKIHWEEKKSNTILGSQEGNTMKTNDTYMK |
| | | FSWLTVPEKSLDKEHRCIVRHENNKNGVDQEIIFPPIKTDVITM |
| 96 | TRD S4 CDR3 | CALGGLGWGYAGDEGGGKLTDKLIF |
| 97 | TRD S4 | MVFSSLLCVFVAFSYSGSSVAQKVTQAQSSVSMPVRKAVTLNCLYETSW |
| | (Constant region) | WSYYIFWYKQLPSKEMIFLIRQGSDEQNAKSGRYSVNFKKAAKSVALTISA |
| | | LQLEDSAKYFCALGGLGWGYAGDEGGGKLTDKLIFGKGTRVTVEPR |
| 98 | TRG S4 CDR3 | CALWEVHPNYKKLF |
| 99 | TRG S4 | MVSLLHASTLAVLGALCVYGAGHLEQPQISSTKTLSKTARLECVVSGITISA |
| | (Constant region) | TSVYWYRERPGEVIQFLVSISYDGTVRKESGIPSGKFEVDRIPETSTSTLTIH |
| | | NVEKQDIATYYCALWEVHPNYKKLFGSGTTLVVTDKQLDADVSPKPTIFLP |
| | | SIAETKLQKAGTYLCLLEKFFPDIIKIHWQEKKSNTILGSQEGNTMKTNDTY |
| | | MKFSWLTVPEESLDKEHRCIVRHENNKNGIDQEIIFPPIKTDVTTV |
| 100 | TRDC | SQPHTKPSVFVMKNGTNVACLVKEFYPKDIRINLVSSKKITEFDPAIVISPSG |
| | | KYNAVKLGKYEDSNSVTCSVQHDNKTVHSTDFEVKTDSTDHVKPKETENT |
| | | KQPSKSCHKPKAIVHTEKVNMMSLTVLGLRMLFAKTVAVNFLLTAKLFFL |
| 101 | TRGC1 | DKQLDADVSPKPTIFLPSIAETKLQKAGTYLCLLEKFFPDVIKIHWQEKKSNT |
| | | ILGSQEGNTMKTNDTYMKFSWLTVPEKSLDKEHRCIVRHENNKNGVDQEII |
| | | FPPIKTDVITMDPKDNCSKDANDTLLLQLTNTSAYYMYLLLLLKSVVYFAIIT |
| | | CCLLRRTAFCCNGEKS |
| 102 | TRGC2 | DKQLDADVSPKPTIFLPSIAETKLQKAGTYLCLLEKFFPDIIKIHWQEKKSNTI |
| | | LGSQEGNTMKTNDTYMKFSWLTVPEESLDKEHRCIVRHENNKNGIDQEIIF |
| | | PPIKTDVTTVDPKYNYSKDANDVITMDPKDNWSKDANDTLLLQLTNTSAYY |
| | | TYLLLLLKSVVYFAIITCCLLRRTAFCCNGEKS |
| 123 | TRD CDR3 X5 | CALGDPYGWGIRNTDKLIF |
| 124 | TRD X5 | MVFSSLLCVFVAFSYSGSSVAQKVTQAQSSVSMPVRKAVTLNCLYETSW |
| | | WSYYIFWYKQLPSKEMIFLIRQGSDEQNAKSGRYSVNFKKAAKSVALTISA |
| | | LQLEDSAKYFCALGDPYGWGIRNTDKLIFGKGTRVTVEPR |
| 125 | TRG CDR3 X5 | CATWDRPPTGWFKIF |
| 126 | TRG X5 | MRWALLVLLAFLSPASQKSSNLEGGTKSVTRPTRSSAEITCDLTVINAFYIH |
| | | WYLHQEGKAPQRLLYYDVSNSKDVLESGLSPGKYYTHTPRRWSWILILRN |
| | | LIENDSGVYYCATWDRPPTGWFKIFAEGTKLIVTSP |
| 127 | TRD CDR3 X1 | CALGELAFLLLTGGSDKLIF |
| 128 | TRD X1 | MVFSSLLCVFVAFSYSGSSVAQKVTQAQSSVSMPVRKAVTLNCLYETSW |
| | | WSYYIFWYKQLPSKEMIFLIRQGSDEQNAKSGRYSVNFKKAAKSVALTISA |
| | | LQLEDSAKYFCALGELAFLLLTGGSDKLIFGKGTRVTVEPR |
| 129 | TRG CDR3 X1 | CATWVNQRGSDWIKTF |
| 130 | TRG X1 | MVLALALLLAFLPPASQKSSNLEGRTKSVTRPTGSSAVITCDLPVENAVYTH |
| | | WYLHQEGKAPQRLLYYDSYNSRVVLESGISREKYHTYASTGKSLKFILENLI |
| | | ERDSGVYYCATWVNQRGSDWIKTFAKGTRLIVTSP |
| 131 | TRD CDR3 X2 | CAFPPSDWDWGVPWSRGALYTDKLIF |
| 132 | TRD X2 | MILTVGFSFLFFYRGTLCDKVTQSSPDQTVASGSEVVLLCTYDTVYSNPDL |
| | | FWYRIRPDYSFQFVFYGDNSRSEGADFTQGRFSVKHILTQKAFHLVISPVR |
| | | TEDSATYYCAFPPSDWDWGVPWSRGALYTDKLIFGKGTRVTVEPR |
| 133 | TRG CDR3 X2 | CATWDGHPNYYKKLF |
| 134 | TRG X2 | MQWALAVLLAFLSPASQKSSNLEGRTKSVIRQTGSSAEITCDLAEGSNGYI |
| | | HWYLHQEGKAPQRLQYYDSYNSKVVLESGVSPGKYYTYASTRNNLRLILR |
| | | NLIENDSGVYYCATWDGHPNYYKKLFGSGTTLVVT |
| 135 | TRD CDR3 X3 | CALGELPLSLTQNWGEYKLIF |
| 136 | TRD X3 | MVFSSLLCVFVAFSYSGSSVAQKVTQAQSSVSMPVRKAVTLNCLYETSW |
| | | WSYYIFWYKQLPSKEMIFLIRQGSDEQNAKSGRYSVNFKKAAKSVALTISA |
| | | LQLEDSAKYFCALGELPLSLTQNWGEYKLIFGKGTRVTVEPR |
| 137 | TRG CDR3 X3 | CATWDLNYYKKLF |
| 138 | TRG X3 | MQWALAVLLAFLSPASQKSSNLEGRTKSVIRQTGSSAEITCDLAEGSNGYI |
| | | HWYLHQEGKAPQRLQYYDSYNSKVVLESGVSPGKYYTYASTRNNLRLILR |
| | | NLIENDSGVYYCATWDLNYYKKLFGSGTTLVVT |
| 139 | TRD CDR3 X4 | CALGEPLPTIWDKLIF |
| 140 | TRD X4 | MVFSSLLCVFVAFSYSGSSVAQKVTQAQSSVSMPVRKAVTLNCLYETSW |
| | | WSYYIFWYKQLPSKEMIFLIRQGSDEQNAKSGRYSVNFKKAAKSVALTISA |
| | | LQLEDSAKYFCALGEPLPTIWDKLIFGKGTRVTVEPR |
| 141 | TRG CDR3 X4 | CATWDRRQNYYKKLF |
| 142 | TRG X4 | MRWALLVLLAFLSPASQKSSNLEGRTKSVTRQTGSSAEITCDLTVTNTFYIH |
| | | WYLHQEGKAPQRLLYYDVSTARDVLESGLSPGKYYTHTPRRWSWILRLQ |
| | | NLIENDSGVYYCATWDRRQNYYKKLFGSGTTLVVT |
| 143 | TRD CDR3 X6 | CALGESLPSLAGYPPVPDKLIF |
| 144 | TRD X6 | MVFSSLLCVFVAFSYSGSSVAQKVTQAQSSVSMPVRKAVTLNCLYETSW |
| | | WSYYIFWYKQLPSKEMIFLIRQGSDEQNAKSGRYSVNFKKAAKSVALTISA |
| | | LQLEDSAKYFCALGESLPSLAGYPPVPDKLIFGKGTRVTVEPR |
| 145 | TRG CDR3 X6 | CATWDRPWIKTF |
| 146 | TRG X6 | MRWALLVLLAFLSPASQKSSNLEGRTKSVTRQTGSSAEITCDLTVTNTFYIH |
| | | WYLHQEGKAPQRLLYYDVSTARDVLESGLSPGKYYTHTPRRWSWILRLQ |
| | | NLIENDSGVYYCATWDRPWIKTFAKGTRLIVTSP |
| 147 | TRD CDR3 X7 | CALGAPGIRLITDKLIF |
| 148 | TRD X7 | MVFSSLLCVFVAFSYSGSSVAQKVTQAQSSVSMPVRKAVTLNCLYETSW |
| | | WSYYIFWYKQLPSKEMIFLIRQGSDEQNAKSGRYSVNFKKAAKSVALTISA |
| | | LQLEDSAKYFCALGAPGIRLITDKLIFGKGTRVTVEPR |
| 149 | TRG CDR3 X7 | CATWDFYKKLF |
| 150 | TRG X7 | MQWALAVLLAFLSPASQKSSNLEGRTKSVIRQTGSSAEITCDLAEGSNGYI |
| | | HWYLHQEGKAPQRLQYYDSYNSKVVLESGVSPGKYYTYASTRNNLRLILR |
| | | NLIENDSGVYYCATWDFYKKLFGSGTTLVVT |
| 166 | TRG CDR1 - cl5 | GITISATS |
| 167 | TRG CDR2 - cl5 | ISYDGTV |
| 168 | TRD CDR1 - cl5 | GEAIGNYY |
| 169 | TRG CDR1 - d25 | EGSTGY |
| 170 | TRG CDR2- d25 | YDSYTSSV |
| 171 | TRD CDR1 - d25 | NSMFDY |
| 172 | TRD CDR2 - d25 | ISSIKDK |

As described herein, the nucleic acids encoding one or more of a γ-chain and/or one or more of a δ-chain can be placed in any order in the polynucleotide with respect to each other, the CAR, and other components for example cis-acting elements of the polynucleotide described herein. In some preferred aspects the order of the nucleic acids on the polynucleotide may be nucleic acid encoding γ-chain/s and derivatives, variations or fragments thereof, a nucleic acid encoding a CAR, and a nucleic acid encoding a δ-chain/s and derivatives, variations or fragments thereof, and with various cis-acting components present at one or more locations. In some aspects the order may be a nucleic acid encoding a γ-chain/s and derivatives, variations or fragments thereof, a nucleic acid encoding a δ-chain/s and derivatives, variations or fragments thereof, and a nucleic acid encoding a CAR, and with various cis-acting components present at one or more locations as necessary. In some aspects the order may be a nucleic acid encoding a δ-chain/s and derivatives, variations or fragments thereof, a nucleic acid encoding a γ-chain/s and derivatives, variations or fragments thereof, and a nucleic acid encoding a CAR, and with various cis-acting components present at one or more locations as necessary. In some aspects the order may be a nucleic acid encoding a CAR, a nucleic acid encoding a γ-chain/s and derivatives, variations or fragments thereof, and a nucleic acid encoding a δ-chain/s and derivatives, variations or fragments thereof with various cis-acting components present at one or more locations as necessary. In some aspects the order may be a nucleic acid encoding a CAR, a nucleic acid encoding a δ-chain/s and derivatives, variations or fragments thereof, and a nucleic acid encoding a γ-chain/s and derivatives, variations or fragments thereof, and with various cis-acting components present at one or more locations as necessary. In some aspects the current disclosure encompasses all functional orders of cistrons in the multicistronic polynucleotide such that the expression of CAR is maximized.

iii. Promoters and Other Regulatory Sequences

As used herein, a regulatory sequence refers to any genetic element that is known to the skilled person to drive or otherwise regulate expression of nucleic acids in a cell. Such sequences include without limitation promoters, transcription terminators, enhancers, repressors, silencers, kozak sequences, polyA sequences, and the like. A regulatory sequence can, for example, be inducible, non-inducible, constitutive, cell-cycle regulated, metabolically regulated, and the like. A regulatory sequence may be a promoter.

In some aspects, the promoter sequence is selected from the group consisting of EF1α, MSCV, EF1 alpha-HTLV-1 hybrid promoter, Moloney murine leukemia virus (MoMuLV or MMLV), Gibbon Ape Leukemia virus (GALV), murine mammary tumor virus (MuMTV or MMTV), Rous sarcoma virus (RSV), MHC class II, clotting Factor IX, insulin promoter, PDX1 promoter, CD11, CD4, CD2, gp47 promoter, PGK, Beta-globin, UbC, and MND, preferably from MSCV, MMLV, EF1α, and MND. In some aspects, the promoter sequence is a derivative sequence (i.e. variant sequence) of a promoter sequence described herein. In some embodiments, a promoter sequence comprises a sequence having at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity with any one of SEQ ID Nos: 103-106. Examples of promoters are further described in Poletti and Mavilio (2021), Viruses 13:8; 1526, Kuroda et al. (2008), J Gene Med 10 (11): 1163-1175, Milone et al. (2009), Mol Ther 17:8; 1453-1464, and Klein et al. (2008), J Biomed Biotechnol 683505, all of which are incorporated herein by reference in their entireties.

TABLE H
Promoter sequences

| SEQ | | |
|---|---|---|
| ID | | |
| NO | Name | SEQUENCE |
| 103 | MSCV | tgaaagaccccacctgtaggtttggcaagctagcttaagtaacgccattttgcaaggcatggaaaatacataactgagaatag |
| | promoter | agaagttcagatcaaggttaggaacagagagacagcagaatatgggccaaacaggatatctgtggtaagcagttcctgccc |
| | | cggctcagggccaagaacagatggtccccagatgcggtcccgccctcagcagtttctagagaaccatcagatgtttccaggg |
| | | tgccccaaggacctgaaaatgaccctgtgccttatttgaactaaccaatcagttcgcttctcgcttctgttcgcgcgtttctg |
| | | ctccccgagctcaataaaagagcccacaacccctcact |
| 104 | MMLV | aatgaaagaccccacctgtaggtttggcaagctagcttaagtaacgccattttgcaaggcatggaaaaatacataactgaga |
| | promoter | atagaaaagttcagatcaaggtcaggaacagatggaacagctgaatatgggccaaacaggatatctgtggtaagcagttcc |
| | | tgccccggctcagggccaagaacagatggaacagctgaatatgggccaaacaggatatctgtggtaagcagttcctgcccc |
| | | ggctcagggccaagaacagatggtccccagatgcggtccagccctcagcagtttctagagaaccatcagatgtttccagggt |
| | | gccccaaggacctgaaatgaccctgtgccttatttgaactaaccaatcagttcgcttctcgcttctgttcgcgcgcttatgct |
| | | ccccgagctcaataaaagagcccacaacccctcactcggggcgccagtcctccgattgactgagtcgcccgggtacccgtgta |
| | | tccaataaaccctcttgcagttgcatccgacttgtggtctcgctgttccttgggagggtctcctctgagtgattgactacccg |
| | | tcagcgggggtctttcatt |
| 105 | EF1α | gctccggtgcccgtcagtgggcagagcgcacatcgcccacagtccccgagaagttggggggaggggtcggcaattgaacc |
| | promoter | ggtgcctagagaaggtggcgcggggtaaactgggaaagtgatgtcgtgtactggctccgcctttttcccgaggggggggag |
| | | aaccgtatataagtgcagtagtcgccgtgaacgttctttttcgcaacgggtttgccgccagaacacaggtaagtgccgtgtgt |
| | | ggttcccgcgggcctggcctctttacgggttatggcccttgcgtgccttgaattacttccacgcccctggctgcagtacgtga |
| | | ttcttgatcccgagcttcgggttggaagtgggtgggagagttcgaggccttgcgcttaaggagccccttcgcctcgtgcttga |
| | | gttgaggcctggcttgggcgctggggccgccgcgtgcgaatctggtggcaccttcgcgcctgtctcgctgctttcgataagtc |
| | | tctagccatttaaaatttttgatgacctgctgcgacgctttttttctggcaagatagtcttgtaaatgcgggccaagatctgc |
| | | acactggtatttcggtttttggggccgcgggcggcgacggggcccgtgcgtcccagcgcacatgttcggcgaggcggggcctg |
| | | cgagcgcggccaccgagaatcggacgggggtagtctcaagctggccggcctgctctggtgcctggcctcgcgccgccgtgtat |
| | | cgccccgccctgggcggcaaggctggcccggtcggcaccagttgcgtgagcggaaagatggccgcttcccggccctgctgcag |
| | | ggagctcaaaatggaggacgcggcgctcgggagagcggggggtgagtcacccacacaaaggaaaagggcctttccgtcctcag |
| | | ccgtcgcttcatgtgactccacggagtaccgggcgccgtccaggcacctcgattagttctcgagcttttggagtacgtcgtct |
| | | ttaggttggggggaggggttttatgcgatggagtttccccacactgagtgggtggagactgaagttaggccagcttggcactt |
| | | gatgtaattctccttggaatttgccctttttgagtttggatcttggttcattctcaagcctcagacagtggttcaaagttttt |
| | | ttcttccatttcaggtgtcgtga |
| 106 | MND | tttatttagtctccagaaaaaggggggaatgaaagaccccacctgtaggtttggcaagctaggatcaaggttaggaacagag |
| | promoter | agacagcagaatatgggccaaacaggatatctgtggtaagcagttcctgccccggctcagggccaagaacagttggaaca |
| | | gcagaatatgggccaaacaggatatctgtggtaagcagttcctgccccggctcagggccaagaacagatggtccccagatg |
| | | cggtcccgccctcagcagtttctagagaaccatcagatgtttccagggtgccccaaggacctgaaatgaccctgtgccttat |
| | | ttgaactaaccaatcagttcgcttctcgcttctgttcgcgcgcttctgctccccgagctcaataaaagagccca |

iv. Cis-Acting Regulatory Elements

Cis-acting regulatory elements as described herein are facilitators of co-expression and include sequences that ensure that the component nucleic acid sequences (for example, the nucleic acid encoding the heterodimeric receptor monomers and the nucleic acid encoding the CAR as described herein) are translated from the single mRNA transcribed from the polynucleotide. A cis-acting regulatory element may be, for example, selected from (but is not limited to) an internal ribosome entry site (IRES) sequence or a sequence encoding a 2A-self cleaving peptide. In some embodiments, the nucleotide sequence inserted between each of the nucleic acids is a sequence encoding a 2A self-cleaving peptide or is an IRES sequence. An IRES sequence functions by allowing the assembly of a new translation initiation complex after the ribosome dissociates from the mRNA following the synthesis of the first polypeptide. Suitable IRES sequences will be known to the skilled person and examples are further available in public databases such as IRESite: The database of experimentally verified IRES structures, described in Mokrejš et al., Nucleic Acids Res. 2006; 34 (Database issue): D125-D130, which is incorporated herein by reference in its entirety. Non-limiting examples of IRES's known in the art include Picornavirus IRES, Apthovirus IRES, Hepatitis A IRES, Pestivirus IRES, and Hepesvirus IRES.

In some aspects, the nucleotide sequence inserted between each of the nucleic acids is a sequence encoding a 2A self-cleaving peptide. 2A self-cleaving peptides (abbreviated herein as “2A peptides”) may be advantageous for expression of multicistronic polynucleotides described herein due to their small size and self-cleavage ability, which allows for facilitation of polypeptide co-expression. 2A peptides are typically composed of 16-22 amino acids and originate from viral RNA. 2A peptide-mediated polypeptide cleavage is typically triggered by ribosomal skipping of the peptide bond between the proline (P) and glycine (G) in the C-terminal of a 2A peptide, resulting in the polypeptide located upstream of the 2A peptide to have extra amino acids on its C-terminal end while the peptide located downstream the 2A peptide has an extra proline on its N-terminal end. Examples of nucleic acid sequences encoding 2A peptides may be found in Xu Y., et al (2019), and Pincha M., et al, (2011) (supra). Non-limiting examples of suitable 2A peptides are F2A (2A peptide derived from the foot-and-mouth disease virus), E2A (2A peptide derived from the equine rhinitis virus), P2A (2A peptide derived from the porcine teschovirus-1), or T2A (2A peptide derived from the Thosea asigna virus). In some embodiments, the 2A self-cleaving peptide is a F2A peptide. In some embodiments, the 2A self-cleaving peptide is an E2A peptide. In some embodiments, the 2A self-cleaving peptide is a P2A peptide. In some embodiments, the 2A self-cleaving peptide is a T2A peptide. The skilled person understands that a polynucleotide described herein may also comprise nucleotide sequences encoding different 2A self-cleaving peptides. As a non-limiting example, in a tricistronic construct, a P2A peptide-encoding sequence may be inserted between the nucleic acid encoding the first and the second polypeptide, and a T2A peptide-encoding sequence may be inserted between the nucleic acid encoding the second and third polypeptide. Accordingly, polynucleotides comprising nucleotide sequences encoding multiple different 2A self-cleaving peptides are also provided. An exemplary polynucleotide comprises a P2A peptide-encoding sequence and a T2A peptide-encoding sequence. A further exemplary polynucleotide comprises a nucleotide sequence encoding a 2A self-cleaving peptide having at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity with SEQ ID NO: 108 or 110 or comprises a nucleotide sequence encoding a 2A self-cleaving peptide represented by an amino acid sequence having an identity or a similarity of at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% with SEQ ID NO: 107 or 109.

TABLE I
Sequences for cis-acting regulatory elements

| SEQ | | |
|---|---|---|
| ID | | |
| NO | Name | SEQUENCE |
| 107 | P2A | ATNFSLLKQAGDVEENPGP |
| 108 | P2A | gccaccaatttcagcctgctgaaacaggctggcgacgtg |
| | DNA | gaagagaaccctgggccc |
| 109 | T2A | EGRGSLLTCGDVEENPGP |
| 110 | T2A | gaaggccgcggcagcctgctgacctgcggagacgtggaa |
| | DNA | gaaaaccctggcccg |

In some aspects the polynucleotide provided herein may comprise at least one or at least two cis-acting regulatory elements. In some aspects, at least one cis-acting regulatory element is selected from F2A, E2A, P2A, or T2A. In some aspects at least one cis-acting regulatory element is selected from a Picornavirus IRES, Apthovirus IRES, Hepatitis A IRES, Pestivirus IRES, Hepesvirus IRES. In some aspects the polynucleotide may comprise at least a first cis-acting regulatory element and a second cis-acting regulatory element such that the first and the second cis-acting regulatory elements are each independently selected from the group consisting of F2A, E2A, P2A, T2A or any combination thereof. In some aspects the polynucleotide may comprise at least a first cis-acting regulatory element and a second cis-acting regulatory elements such that the first and second cis-acting regulatory elements are each independently selected from the group consisting of Picornavirus IRES, Apthovirus IRES, Hepatitis A IRES, Pestivirus IRES, Hepesvirus IRES, and combinations thereof.

v. Exemplary Polynucleotides

In accord with the foregoing, certain exemplary polynucleotides are provided that comprise a nucleotide sequence encoding a polypeptide having at least 60%, at least 65%, at least 70%, at least 75%, at least 80%, at least 85%, at least 90%, at least 95%, at least 97%, at least 98%, at least 99%, or up to 100% sequence identity or similarity with any one of SEQ ID NO: 112, 152, 158, and 174, In various aspects, the polynucleotide may comprise a nucleotide sequence encoding a polypeptide comprising an amino acid sequence selected from SEQ ID NO: 112, 152, 158, and 174. In various embodiments, the polynucleotide may comprise a nucleotide sequence having at least 60%, at least 65%, at least 70%, at least 75%, at least 80%, at least 85%, at least 90%, at least 95%, at least 97%, at least 98%, at least 99%, or up to 100% sequence identity or similarity with any one of SEQ ID NOs: 111, 151 157, and 173. In some aspects, the polynucleotide may comprise a nucleotide sequence comprising any one of SEQ ID NOs; 111, 151, 157 and 173.

### B. Vectors

Compositions of the current disclosure encompass vectors comprising any polynucleotide as described herein. A vector can be any genetic element, e.g., a plasmid, chromosome, virus, transposon, behaving either as an autonomous unit of polynucleotide replication within a cell. (i.e., capable of replication under its own control) or being rendered capable of replication by insertion into a cell chromosome, having attached to it another polynucleotide segment, so as to bring about the replication and/or expression of the attached segment. Suitable vectors include, but are not limited to, plasmids, transposons, bacteriophages and cosmids. Vectors can contain polynucleotide sequences which are necessary to effect ligation or insertion of the vector into a desired host cell and to affect the expression of the attached segment. Such sequences differ depending on the host organism; they include promoter sequences to effect transcription, enhancer sequences to increase transcription, ribosomal binding site sequences and transcription and translation termination sequences. Alternatively, expression vectors can be capable of directly expressing nucleic acid sequence products encoded therein without ligation or integration of the vector into host cell DNA sequences. A vector can comprise a selectable marker gene. In some embodiments, the vector is an “episomal expression vector” or “episome,” which is able to replicate in a host cell and persists as an extrachromosomal segment of DNA within the host cell in the presence of appropriate selective pressure.

A polynucleotide vector useful for the methods and compositions described herein can be a good manufacturing practices (GMP) compatible vector. For example, a GMP vector can be purer than a non-GMP vector. In some cases, purity can be measured by bioburden. For example, bioburden can be the presence or absence of aerobes, anaerobes, sporeformers, fungi, or combinations thereof in a vector composition. In some cases, a pure vector can be endotoxin low or endotoxin free. Purity can also be measured by double-stranded primer-walking sequencing. Plasmid identity can be a source of determining purity of a vector. A GMP vector of the invention can be from 10% to 99% more pure than a non-GMP vector. A GMP vector can be from 10%, 15%, 20%, 25%, 30%, 35%, 40%, 45%, 50%, 55%, 60%, 65%, 70%, 75%, 80%, 85%, 90%, 95%, 96%, 97%, 98%, or 99% more pure than a non-GMP vector as measured by the presence of bioburden, endotoxin, sequencing, or combinations thereof.

In some aspects, the vector comprising the polynucleotide described herein may be a viral vector. A viral vector can be a DNA or an RNA virus, with either episomal or integrated genomes after delivery to the cell. Examples of viral vectors include, but are not limited to, retroviral vectors, lentiviral vectors, adenovirus vectors, poxvirus vectors; herpesvirus vectors and adeno-associated virus (AAV) vectors, helper-dependent adenovirus vectors, hybrid adenovirus vectors, Epstein-Bar virus vectors, herpes simplex virus vectors, hemagglutinating virus of Japan (HVJ) vectors, and Moloney murine leukemia virus vectors.

In accord with the foregoing, certain exemplary vectors are provided that comprise a nucleotide sequence encoding a polypeptide having at least 60%, at least 65%, at least 70%, at least 75%, at least 80%, at least 85%, at least 90%, at least 95%, at least 97%, at least 98%, at least 99%, or up to 100% sequence identity or similarity with any one of SEQ ID NO: 112, 152, 158, and 174, In various aspects, the vector may comprise a nucleotide sequence encoding a polypeptide comprising an amino acid sequence selected from SEQ ID NO: 112, 152, 158, and 174. In various embodiments, the vector may comprise a nucleotide sequence having at least 60%, at least 65%, at least 70%, at least 75%, at least 80%, at least 85%, at least 90%, at least 95%, at least 97%, at least 98%, at least 99%, or up to 100% sequence identity or similarity with any one of SEQ ID NOs: 111, 151, 157 and 173. In some aspects, the vector may comprise a nucleotide sequence comprising any one of SEQ ID NOS; 111, 151, 157 and 173.

### C. Cell Compositions

Engineered cells as disclosed herein comprise at least one polynucleotide and/or vector as described herein, and preferably express the polypeptides encoded by the polynucleotide(s) and/or vector(s). Accordingly, in some aspects the present disclosure provides an engineered cell or populations of such cells that express one or more CAR and monomers of a heterodimeric γδT-cell receptor or functional fragments thereof. Expression of one or more of these proteins in an engineered cell or population thereof can be used as a strategy to overcome limitations that hamper the production and use of engineered cells, for example, low expression, limited cytotoxic effect, limited immune stimulatory effect, limited proliferative ability or lifespan of the engineered cells, limited induction of effector function upon engineered cell recognition of antigen, and engineered cell exhaustion. Within the context of the application, the expression “engineered cell” refers to a cell that has been modified using recombinant DNA technology. In an embodiment, an “engineered cell” has been transformed, modified, or transduced to comprise a heterologous nucleic acid molecule. It should be understood that the term “engineered cell(s)” is used interchangeably herein with the term “modified cell” or “transformed cell” or “transduced cell.” In an embodiment, an engineered cell expresses a protein encoded by the heterologous nucleic acid molecule.

In some aspects the compositions described herein comprise mammalian cells, or cell populations comprising the polynucleotides or vectors described herein. In some aspects the compositions described herein comprise human cells or cell populations comprising the polynucleotides or vectors described herein. In some aspects the cell compositions described herein comprise at least an immune cell comprising the polynucleotides described herein. As used herein, an “immune cell” means any cell of the immune system that originate from a hematopoietic stem cell in the bone marrow, which gives rise to two major lineages, a myeloid progenitor cell (which give rise to myeloid cells such as monocytes, macrophages, dendritic cells, meagakaryocytes and granulocytes) and a lymphoid progenitor cell (which give rise to lymphoid cells such as T cells, B cells and natural killer (NK) cells). Exemplary immune cells include CD4+ T cells, CD8+ T cells, CD4− CD8− double negative T cells, γδ T cells, regulatory T cells, natural killer cells, and dendritic cells. Macrophages and dendritic cells may be referred to as “antigen presenting cells” or “APCs,” which are specialized cells that can activate T cells when a major histocompatibility complex (MHC) receptor on the surface of the APC interacts with a TCR on the surface of a T cell. In some aspects, an engineered immune cell provided herein may comprise additional edits and or modifications in comparison to naturally occurring counterparts of the same cell.

In a non-limiting, exemplary aspect, a cell that comprises a polynucleotide and/or a vector as described herein, and preferably expresses the polypeptides encoded by the polynucleotides and/or vector, is a T cell. A “T cell” is an immune cell that matures in the thymus and produces T cell receptors (TCRs). T cells can be naïve (not exposed to antigen; increased expression of CD62L, CCR7, CD28, CD3, CD127, and CD45RA, and decreased expression of CD45RO as compared to TCM), memory T cells I (antigen-experienced and long-lived), and effector cells (antigen-experienced, cytotoxic). TM can be further divided into subsets of central memory T cells (TCM, increased expression of CD62L, CCR7, CD28, CD127, CD45RO, and CD95, and decreased expression of CD54RA as compared to naïve T cells) and effector memory T cells (TEM, decreased expression of CD62L, CCR7, CD28, CD45RA, and increased expression of CD127 as compared to naïve T cells or TCM). Effector T cells (TE) refers to a antigen-experienced CD8+ cytotoxic T lymphocytes that has decreased expression of CD62L, CCR7, CD28, and are positive for granzyme and perforin as compared to TCM. In some aspects, an engineered cell of the disclosure is a T cell, preferably an aβT cell or a yoT cell, more preferably an aβT cell. In some cases, an engineered cell is a primary cell. In some cases, an engineered cell is not a primary cell. In some aspects the engineered T-cells provided herein may comprise additional edits or modifications compared to their naturally occurring counterparts.

As used herein a “TEG” is a T cell engineered to express a defined γδ TCR as disclosed herein. In a preferred non-limiting example, a TEG can be an alpha-beta T cell that is engineered to express a defined yo TCR. In another preferred non-limiting example, a TEG can be an alpha-beta T cell that is engineered to express a defined γδ TCR and a defined CAR (e.g., a BCMA-CAR, a CEA-CAR, or a CD19 CAR as described herein).

In some aspects the current disclosure encompasses a TEG comprising the polynucleotide provided herein. In some aspects there is provided a TEG comprising a vector comprising the polynucleotide provided herein. In an aspect, this cell expresses the provided chimeric proteins. In an aspect, a population of cells is provided comprising such a TEG.

In some aspects the TEGs provided herein exhibit significantly enhanced expression of CARs when the encoding polynucleotide is polycistronic and further encodes monomers of γδ TCR, and when the nucleic acids encoding the CAR and the monomers are operably linked to the same promoter sequence. In some aspects the TEG exhibits increased CAR expression level compared to a TEG without γδ TCR. Expression of a heterodimeric receptor and the CAR may be assessed by any standard technique available to the skilled person discussed earlier herein, such as flow cytometry, FACS, and the like. Further non-limiting examples are provided in the examples.

In some aspects, in addition or alternatively to increased CAR expression, the TEGs can exhibit any one or more of increased cytotoxicity, antitumor activity, and/or tumor cell killing, and/or proliferation, cellular survival, or persistence, as compared to T cells that do not co-express said γδT-cell receptor and said CAR or express the CAR alone. Through the application, the wording “target biological outcome” may be replaced by “biological parameter and/or biological function” and comprises one or more of cytotoxicity, antitumor activity, and/or tumor cell killing, and/or proliferation, cellular survival, or persistence. A target biological outcome (i.e. a biological parameter and/or biological function) can be or can comprise, for example, cellular proliferation, cellular survival, magnitude of immune effector function, duration of immune effector function, cytotoxic effects on a cell (e.g., a cancer cell), production of inflammatory mediators, an anti-cancer immune response, cellular differentiation, cellular dedifferentiation. In an aspect, the biological parameter and/or function is selected from proliferation, cellular survival, cytotoxicity, antitumor activity, persistence and/or tumor cell killing and/or proliferation. The methods of determining these biological parameters and functions are well known in the art.

A target biological outcome or biological parameter and/or function can include a cytotoxic response, e.g., against cancer cell. A cytotoxic response may be determined directly (e.g., by measuring cell lysis or survival of target cells). Alternatively, or in addition, a cytotoxic response may be determined by measuring the production of molecules associated with such a response, for example a production of a cytokine such as interferon gamma (IFNγ). Suitable measurement assays, for example luminescence assays to determine cytotoxicity and ELISA to determine IFNγ production are known to the skilled person and further non-limiting examples are provided in the experimental section. In some aspects the cytotoxic response can be measured by any of the methods known in the art some of which are provided herein in the examples (e.g., a luciferase assay). As detailed in the Examples below, a luciferase cytotoxicity assay can comprise use of a target cell population (e.g., an immortalized cancer cell line) that is genetically engineered to express a luciferase, which becomes detectable upon cell lysis. Therefore, a cytotoxic response may be easily determined by monitoring a fluorescent (luciferase) signal. Exemplary luciferase expressing target cell populations that may be used in these methods can include RPMI-8226 LucTOM cells, Daudi cells, MM1S tumor cells and HT-29 LucTOM cells.

In some aspects, a target biological function of an engineered T-cell is elicited by or directed against cells that express or present an antigen recognized by the antigen-recognition domain of the CAR expressed in conjunction with the TCR. For example, in some embodiments, where a target biological function comprises a cytotoxic response against cancer cells, the engineered cells can kill cancer cells based on recognition of an antigen by an antigen-recognition domain.

In some aspects, upon exposure to a cell expressing the antigen, the target biological function of the engineered cell is increased by at least 10%, at least 20%, at least 30%, at least 40%, at least 50%, at least 60%, at least 70%, at least 80%, at least 90%, at least 2-fold, at least 3-fold, at least 5-fold, at least 10-fold, at least 20-fold, at least 50-fold, at least 100-fold, or at least 1000-fold longer than a corresponding cell that does not express the CAR and TCR monomers or fragments thereof or expresses the CAR alone.

In an aspect, upon exposure to a cell expressing the antigen, at least one or more of the proliferation, cellular survival, cytotoxicity, antitumor activity, persistence and/or tumor cell killing of the engineered cell is increased by at least 10%, at least 20%, at least 30%, at least 40%, at least 50%, at least 60%, at least 70%, at least 80%, at least 90%, at least 2-fold, at least 3-fold, at least 5-fold, at least 10-fold, at least 20-fold, at least 50-fold, at least 100-fold, or at least 1000-fold compared to a corresponding cell that does not co-express the proteins provided herein or expresses the CAR alone.

### Population of Engineered Cells

In some aspects, the current disclosure also encompasses a plurality, i.e., a population of engineered T-cells expressing the same or different polynucleotides provided in the current disclosure. For example, the current disclosure also encompasses mixtures of TEGs co-expressing the same CAR or different CARs with the same γδ TCR monomers, or different combinations of monomers or variants or fragments thereof. In some aspects the population of cells provided herein individually or together exhibit enhanced biological function in comparison to a population of cells expressing the respective CARs alone. In some aspects these cell populations may comprise cells having one or more additional modifications that improve biological function.

In some aspects, the current disclosure also encompasses a population of cells at least one cell of which comprises a polynucleotide disclosed here. In some aspects the population may only comprise engineered cell or plurality of engineered cells as provided herein. In some aspects of cells may further comprise additional cells not comprising the polynucleotide provided herein. For example, in certain aspects, the cell population comprises γδ T cells, αβ T cells and NK cells and at least a portion of the T cells (γδ T cells and/or αβ T cells) comprise one or more polynucleotides provided herein. In some aspects, a cell population as described herein comprises engineered T cells and other engineered or non-engineered immune system cells. In some exemplary aspects the cell population comprises at least 5% to 10%, or 10% to 20%, or 20% to 30%, or 30% to 40%, or 40% to 50%, or 50% to 60%, or 60% to 70%, or 70% to 80%, or 80% to 90%, or 90% to 100% of the engineered cells provided herein.

In an aspects, a population of cells that co-express the CAR and γδ TCR monomers or fragments thereof exhibit enhancement of at least one or more of: proliferation, cellular survival, cytotoxicity, antitumor activity, persistence and/or tumor cell killing ability, by at least 10%, at least 20%, at least 30%, at least 40%, at least 50%, at least 60%, at least 70%, at least 80%, at least 90%, at least 2-fold, at least 3-fold, at least 5-fold, at least 10-fold, at least 20-fold, at least 50-fold, at least 100-fold, or at least 1000-fold as compared to a corresponding population of cells that do not express the chimeric protein.

### D. Pharmaceutical Compositions

In some aspects the current disclosure comprises pharmaceutical compositions for use in the methods of treatment provided herein. In some aspects the engineered cells and cell populations described herein are formulated into a pharmaceutical composition that can be administered to a subject in need thereof. In some embodiments, pharmaceutical compositions include a vector for introduction of a polynucleotide into cells in vitro, ex vivo, or in vivo. In some aspects, viral vectors containing the disclosed polynucleotide are administered to a subject with cancer.

In some aspects the polynucleotide, a vector comprising said polynucleotide, a cell comprising, preferably expressing proteins encoded by the polynucleotide or a population of cells comprising said cell are for use for treating a disease or a condition. In some aspects the polynucleotide, a vector comprising said polynucleotide, a cell comprising, preferably expressing proteins encoded by the polynucleotide or a population of cells comprising said cell is provided for the manufacture of a medicament or pharmaceutical composition for treating a disease or condition.

In some instances, pharmaceutical compositions are formulated in a conventional manner using one or more physiologically acceptable carriers including excipients and auxiliaries which facilitate processing of the active compounds into preparations which can be used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

In certain embodiments, compositions can also include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

In some aspects, compositions can also include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

The pharmaceutical compositions described herein can be administered by any suitable administration route, including but not limited to, parenteral (e.g., intravenous, intratumoral, subcutaneous, intramuscular, intracerebral, intracerebroventricular, intra-articular, intraperitoneal, or intracranial), intranasal, buccal, sublingual, oral, or rectal administration routes. In some instances, the pharmaceutical composition is formulated for parenteral (e.g., intravenous, intratumoral, subcutaneous, intramuscular, intracerebral, intracerebroventricular, intra-articular, intraperitoneal, or intracranial) administration.

The pharmaceutical compositions described herein are formulated into any suitable dosage form, including but not limited to, aqueous dispersions, liquids, gels, syrups, elixirs, slurries, suspensions and the like, for administration to a subject to be treated. In some embodiments, the pharmaceutical compositions are formulated into solutions (for example, for IV administration). In some cases, the pharmaceutical composition is formulated as an infusion. In some cases, the pharmaceutical composition is formulated as an injection.

Parenteral administration can be, for example, by bolus injection or by gradual perfusion over time. Administration can also be by surgical deposition of a bolus or pellet of cells or positioning of a medical device.

In some aspects, pharmaceutical compositions described herein are formulated into solid oral dosage forms, aerosols, controlled release formulations, fast melt formulations, effervescent formulations, lyophilized formulations, tablets, powders, pills, dragees, capsules, delayed release formulations, extended-release formulations, pulsatile release formulations, multiparticulate formulations, and mixed immediate release and controlled release formulations. In some embodiments, the pharmaceutical compositions are formulated into capsules.

The pharmaceutical solid dosage forms described herein optionally include a compound described herein and one or more pharmaceutically acceptable additives such as a compatible carrier, binder, filling agent, suspending agent, flavoring agent, sweetening agent, disintegrating agent, dispersing agent, surfactant, lubricant, colorant, diluent, solubilizer, moistening agent, plasticizer, stabilizer, penetration enhancer, wetting agent, anti-foaming agent, antioxidant, preservative, or one or more combination thereof.

A therapeutically effective amount of a composition of the disclosure can be administered to a subject. A “therapeutically effective amount” can refer to an amount effective, at dosages and for periods of time necessary, to achieve a desired therapeutic result. The therapeutically effective amount can vary according to factors such as the disease state, age, sex, and weight of the individual, and the ability of the inventive nucleic acid sequences to elicit a desired response in the individual.

In some aspects the pharmaceutical composition comprises cells described herein. Cells administered to a subject in need thereof can be autologous to the subject. Cells administered to a subject in need thereof can be allogeneic to the subject, for example, fully HLA-matched, HLA matched at 1, 2, 3, 4, 5, 6, 7, or 8 HLA alleles, or at least 1, at least 2, at least 3, at least 4, at least 5, at least 6, at least 7, or at least 8 HLA alleles. Cells administered to a subject in need thereof can be haploidentical to the subject. Cells administered to a subject in need thereof can be from a donor that is related to the subject. Cells administered to a subject in need thereof can be from a donor that is not related to the subject.

### III. Methods

In some aspects the current disclosure also comprises methods of increasing expression or function of a CAR. In some aspects the current disclosure further encompasses methods of enhancing the biological function of a cell comprising the compositions provided herein. In some aspects the current disclosure further encompasses methods of enhancing the biological function of a cell population comprising the compositions provided herein. In some aspects the current disclosure further encompasses methods of using the compositions provided herein for use in treatment of disease or conditions.

A. Method for Increasing Expression and/or Function of a CAR in a Cell

In some aspects the current disclosure comprises methods of increasing expression and/or function of a CAR in a cell, the method comprising introducing the polynucleotide or vectors provided herein to a cell and expressing the encoded polypeptide in the cell.

In some aspects the current disclosure also encompasses methods of making engineered cells disclosed herein, using the polynucleotides and vectors provided.

Cells can be obtained from any suitable source for the generation of engineered cells. Cells can be primary cells. Cells can be recombinant cells. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. Cells can be derived from a healthy donor, from a patient diagnosed with cancer, or from a patient diagnosed with an infection. Cells can also be obtained from a cell therapy bank. Cells can also be obtained from whole food, apheresis, or a tumor sample of a subject. A cell can be a tumor infiltrating lymphocytes (TIL). In some cases, an apheresis can be a leukapheresis.

A desirable cell population can also be selected prior to modification. A selection can include at least one of: magnetic separation, flow cytometric selection, antibiotic selection. The one or more cells can be any blood cells, such as peripheral blood mononuclear cell (PBMC), lymphocytes, monocytes or macrophages. The one or more cells can be any immune cells such as a lymphocyte, a T cell, an alpha-beta T cell, a gamma-delta T cell, a Jurkat cell, CD4+ T cell, CD8+ T cell, a T effector cell, a lymphocyte, a B cell, an NK cell, an NKT cell, a myeloid cell, a monocyte, a macrophage, or a neutrophil, preferably an alpha-beta T cell or a gamma-delta T cell, more preferably an alpha beta T cell.

In a further aspect of the current disclosure cells can be cultured, expanded and activated using methods known in the art. For instance, conditions appropriate for T cell culture can include an appropriate media (e.g., Minimal Essential Media or RPMI Media 1640, TexMACS (Miltenyi) or, X-vivo 5, (Lonza)) that may contain factors necessary for proliferation and viability, including serum. In some cases, serum-free medium is used. In an aspect, cells can be maintained under conditions necessary to support growth; for example, an appropriate temperature (e.g., 37° C.) and atmosphere (e.g., air plus 5% CO2).

In some aspects, methods of making engineered cells can comprise stimulation, such as by contact with an anti-CD3 antibody or antigen-binding fragment thereof, or an anti-CD2 antibody immobilized on a surface, or by contact with a protein kinase C activator (e.g., bryostatin) sometimes in conjunction with a calcium ionophore. For co-stimulation of an accessory molecule on the surface of the T cells, a ligand that binds the accessory molecule can be used. In some cases, a population of T cells can be CD3-CD28 co-stimulated, for example, contacted with an anti-CD3 antibody and an anti-CD28 antibody, under conditions that can stimulate proliferation of the T cells.

T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005, which are incorporated by reference for such disclosure.

Methods of making engineered cells can comprise the use of a vector described herein to introduce a polynucleotide described herein.

A variety of enzymes can catalyze insertion of foreign DNA into a host genome. Non-limiting examples of gene editing tools and techniques include CRISPR, TALEN, zinc finger nuclease (ZFN), meganuclease, Mega-TAL, and transposon-based systems.

A CRISPR system can be utilized to facilitate insertion of a polynucleotide sequence encoding a membrane protein or a component thereof into a cell genome. For example, a CRISPR system can introduce a double stranded break at a target site in a genome. There are at least five types of CRISPR systems which all incorporate RNAs and CRISPR-associated proteins (Cas). Types I, III, and IV assemble a multi-Cas protein complex that is capable of cleaving nucleic acids that are complementary to the crRNA. Types I and III both require pre-crRNA processing prior to assembling the processed crRNA into the multi-Cas protein complex. Types II and V CRISPR systems comprise a single Cas protein complexed with at least one guiding RNA.

A transposon-based system can be utilized for insertion of a polynucleotide or a component thereof into a genome.

Methods to introduce gene editing components into a cell include, but are not limited to, electroporation, sonoporation, use of a gene gun, lipofection, calcium phosphate transfection, use of dendrimers, microinjection, and use of viral vectors. Viral vector delivery systems can include DNA and RNA viruses, which have either episomal or integrated genomes after delivery to the cell. Examples of viral vectors include, but are not limited to, retroviral vectors, lentiviral vectors, adenovirus vectors, poxvirus vectors; herpesvirus vectors and adeno-associated virus (AAV) vectors, helper-dependent adenovirus vectors, hybrid adenovirus vectors, Epstein-Bar virus vectors, herpes simplex virus vectors, hemagglutinating virus of Japan (HVJ) vectors, and Moloney murine leukemia virus vectors.

In some aspects the engineered cells of the current disclosure exhibit enhanced expression and functionality of CAR when co-expressed with a monomers of a heterodimeric γδT-cell receptor or functional fragments thereof. Methods of assessing the level of expression of CAR are well known in the art. Non-limiting examples of suitable assays are western blotting, FACS, florescence imaging, or ELISA. In some aspects an engineered cell may exhibit at least 10%, at least 20%, at least 30%, at least 40%, at least 50%, at least 60%, at least 70%, at least 80%, at least 90%, at least 2-fold, at least 3-fold, at least 5-fold, at least 10-fold, at least 20-fold, at least 50-fold, at least-100 fold, or at least-1000 fold enhanced expression of CAR compared to a cell expressing the CAR alone.

In some aspects the engineered cells of the current disclosure co-expressing a CAR and monomers of a heterodimeric γδT-cell receptor or functional fragments thereof exhibit enhanced biological functionality in comparison to cells that express the CAR or monomers of a heterodimeric γδT-cell receptor or functional fragments thereof. Biological function can comprise any one or more of increased cytotoxicity, antitumor activity, and/or tumor cell killing, and/or proliferation, cellular survival, or persistence, as compared to T cells that do not co-express said γδT-cell receptor and said CAR. Methods of assessing biological function differ based on the aspects being considered and are well known in the art. For instance, if the desired biological functionality is enhanced in vitro cytotoxicity, methods include 51Cr-release assay, bioluminescent assays, cell-based flow cytometry assay, cytokine release assay, tumor killing assays etc. In some aspects an engineered cell may exhibit at least 10%, at least 20%, at least 30%, at least 40%, at least 50%, at least 60%, at least 70%, at least 80%, at least 90%, at least 2-fold, at least 3-fold, at least 5 fold, at least 10 fold, at least 20 fold, at least 50 fold, at least 100 fold, or at least 1000 enhanced cytotoxicity compared to a cell expressing the CAR alone.

Increased proliferation can be determined by measuring the incorporation of either tritiated thymidine or orotic acid to measure DNA synthesis following ligand binding to the CAR-expressing cells disclosed herein. The incorporation of bromodeoxyuridine into newly synthesized DNA can be measured by immunological staining and the detection of dyes, or by ELISA (Enzyme-linked immunosorbent assay) (Doyle et al., Cell and Tissue Culture: Laboratory Procedures, Wiley, Chichester, England, (1994)). The mitotic index of cells can be determined by staining and microscopy, by the fraction labeled mitoses method or by fluorescence activated cell sorting (FACS) analysis (Doyle et al., supra; Dean, Cell Tissue Kinet. 13:299-308, 1980; Dean, Cell Tissue Kinet. 13:672-681, 1980). The increase in cell size which accompanies progress through the cell cycle can be measured by centrifugal elutriation (Faha et al., J. Virol. 67:2456-2465, 1993). Increases in the number of cells may also be measured by counting the cells, with or without the addition of vital dyes. In addition, signal transduction can be measured by the detection of phosphotyrosine, the in vitro activity of tyrosine kinases from activated cells, c-myc induction, or calcium mobilization.

One measure of T cell activation is the production of cytokines. In some embodiments, CD28 co-stimulation increases cytokine production by increasing transcription of cytokine genes and stabilizing cytokine mRNAs. In other embodiments, CD4+ T cells and CD8+ T cells expressing the CARs disclosed herein have a greater capacity for cytokine production. Specific, non-limiting examples of cytokines include IL-2, IL-4, and γ-IFN.

Animal models can be used to assess in vivo activity of the T-cells. Imaging technologies can be used to evaluate specific trafficking and proliferation of CARs in tumor-bearing animal models. In some aspects any assay including assessment of tumor size, shrinkage, tumor marker assays, metastasis assays can be used. In some aspects the engineered T-cells are at least 2%, at least 5%, at least 10%, at least 20%, at least 30%, at least 40%, at least 50%, at least 60%, at least 70%, at least 90%, at least 100% more effective in inducing a positive biological response to tumors than T-cells expressing the CAR alone.

### B. Methods for Treatment and Use

In some aspects the current disclosure also encompasses methods of using the pharmaceutical compositions provided herein to treat a disease or condition in a subject in need thereof.

As used herein, the term “subject” may include a mammal or a human e.g., humans, other primates, pigs, rodents, such as mice and rats, rabbits, guinea pigs, hamsters, horses, cows, cats, dogs, sheep, chickens and goats. Human and veterinary applications are anticipated by the present disclosure. Both pediatric and adult subjects are included. For example, in any of the methods described herein, the subject can be at least 6 months old (e.g., 6 months or older, 12 months or older, 18 months or older, 2 years or older, 4 years or older, 6 years or older, 10 years or older, 13 years or older, 16 years or older, 18 years or older, 21 years or older, 25 years or older, 30 years or older, 35 years or older, 40 years or older, 45 years or older, 50 years or older, 60 years or older, 65 years or older, 70 years or older, 75 years or older, 80 years or older, 85 years or older, 90 years or older, or 1, 2, 3, 4, 5, 6, 7, 8, 9, 10, 12, 13, 14, 15, 16, 18, 20, 21, 24, 25, 27, 28, 30, 33, 35, 37, 39, 40, 42, 44, 45, 48, 50, 52, 55, 60, 65, 70, 75, 80, 85, 90, 95, 96, 97, 98, 99, 100, 101, 102, 103, 104, or more years old).

In some aspects the subject is suffering from a cancer. Cancers that may be treated include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with the CARs of the disclosure include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included. In one embodiment, the cancer to be treated in a solid tumor, e.g., a solid tumor described herein.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hod′kin's disease, non-Hod′kin's lymphoma (indolent and high grade forms), multiple myeloma, Waldens′rom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, E′ing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, W′Ims' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

As described herein, the disclosure provides methods of treating cancer by providing to the subject in need thereof immune system cells (e.g., T cells) that are engineered to co-express CARs with monomers of a heterodimeric γδT-cell receptor or functional fragments thereof. In one aspect, the immune system cells that are engineered are αβ T-cells. These engineered cells are particularly suitable for adoptive immunotherapy. In some aspect, the current disclosure encompasses use of the methods presented herein to combat any cancer for which an effective CAR is known. In some aspect, the current disclosure encompasses use of the methods presented herein to combat any cancer for which a novel effective CAR is developed. For example, the disclosure provides methods of treating cancer by providing to the subject in need thereof immune cells (e.g., T cells) that are engineered to co-express CD19 CAR, e.g., with an anti-CD19 binding domain known or described herein, wherein the cancer cells express CD19. In one embodiment, the cancer to be treated is ALL (acute lymphoblastic leukemia), CLL (chronic lymphocytic leukemia), DLBCL (diffuse large B-cell lymphoma), MCL (Mantle cell lymphoma, or MM (multiple myeloma).

In some aspects, the disclosure provides methods of treating cancer by providing to the subject in need thereof immune cells (e.g., T cells) that are engineered to co-express BCMA CAR e.g., with an anti-BCMA binding domain known or described herein. In various aspects, the T-cells are engineered to co-express a BCMA CAR (e.g., having least 60%, 70%, 80%, 90%, 95%, or 100% identity or similarity to SEQ ID NO: 29) and a heterodimeric γδT-cell receptor or functional fragments thereof (e.g., a γ902 or γ405 T-cell receptor). In various aspects, the T-cells are engineered to express a polypeptide having least 60%, 70%, 80%, 90%, 95%, or 100% identity or similarity to SEQ ID NO: 112 or 152. The disease associated with BCMA is a hematologic cancer selected from the group consisting of one or more acute leukemias including but not limited to acute myeloid leukemia (AML); myelodysplasia syndrome; myeloproliferative neoplasms; chronic myeloid leukemia (CML); Blastic plasmacytoid dendritic cell neoplasm; multiple myeloma; and to disease associated with BMCA expression including, but not limited to atypical and/or non-classical cancers, malignancies, precancerous conditions or proliferative diseases expressing BCMA; and combinations thereof. In one aspect, the disease associated with BCMA is multiple myeloma. In some aspects, a disease associated with expression of BCMA includes a plasma cell proliferative disorder, e.g., asymptomatic myeloma (smoldering multiple myeloma or indolent myeloma), monoclonal gammapathy of undetermined significance (MGUS), Waldens′rom's macroglobulinemia, plasmacytomas (e.g., plasma cell dyscrasia, solitary myeloma, solitary plasmacytoma, extramedullary plasmacytoma, and multiple plasmacytoma), systemic amyloid light chain amyloidosis, and POEMS syndrome (also known as Crow-Fukase syndrome, Takatsuki disease, and PEP syndrome). In some aspects, a disease associated with expression of BCMA includes a cancer, e.g., a cancer described herein, e.g., a prostate cancer (e.g., castrate-resistant or therapy-resistant prostate cancer, or metastatic prostate cancer), pancreatic cancer, or lung cancer.

In some aspects, the disclosure provides methods of treating cancer by providing to the subject in need thereof immune cells (e.g., T cells) that are engineered to co-express CEA CAR e.g., with an anti-CEA binding domain known or described herein. In various aspects, the T-cells are engineered to co-express a CEA CAR (e.g., having least 60%, 70%, 80%, 90%, 95%, or 100% identity or similarity to SEQ ID NO: 31) and a heterodimeric γδT-cell receptor or functional fragments thereof (e.g., a γ902 or γ455 T-cell receptor). In various aspects, the T-cells are engineered to express a polypeptide having least 60%, 70%, 80%, 90%, 95%, or 100% identity or similarity to SEQ ID NO: 158. The disease associated with CEA is typically a solid tumor cancer selected from the group consisting of colorectal or bowel cancer, prostate cancer, ovarian cancer, lung cancer, thyroid cancer, liver cancer, pancreatic cancer, or breast cancer, or from diseases associated with CEA expression including, but not limited to atypical and/or non-classical cancers, malignancies, precancerous conditions or proliferative diseases expressing CEA; and combinations thereof. In one aspect, the disease associated with CEA is colorectal cancer, a gastric carcinoma, a pancreatic carcinoma, a lung carcinoma, and medullary thyroid carcinoma. In some aspects, a disease associated with expression of CEA includes a cancer, e.g., a cancer described herein, e.g., a prostate cancer (e.g., castrate-resistant or therapy-resistant prostate cancer, or metastatic prostate cancer), pancreatic cancer, a lung cancer, or a colorectal cancer.

In some aspects, engineered cells as disclosed herein which coexpress the desired CAR with monomers of a heterodimeric γδT-cell receptor or functional fragments thereof, can be administered in an amount or dose that is higher, lower or the same than the amount or dosage used for administering a cell, or composition of cells expressing the CAR alone. In some aspects the dosage may be at least 5%, at least 10%, at least 15%, at least 20%, at least 25%, at least 30%, at least 35%, at least 40%, at least 45%, at least 50%, at least 55%, at least 60% lower that the recommended dosage for cells expressing CAR alone for the same patient.

In further aspects, a CAR-expressing cell described herein may be used in a treatment regimen in combination with surgery, chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludarabine, cyclosporin, FK506, rapamycin, mycophenolic acid, steroids, FR901228, cytokines, checkpoint inhibitors such as pembrolizumab (Keytruda), ipilimumab (Yervoy), nivolumab (Opdivo) and atezolizumab (Tecentriq), peptide vaccine, or any combination thereof.

Cells administered to a subject in need thereof can be autologous to the subject. Cells administered to a subject in need thereof can be allogeneic to the subject, for example, fully HLA-matched, HLA matched at 1, 2, 3, 4, 5, 6, 7, or 8 HLA alleles, or at least 1, at least 2, at least 3, at least 4, at least 5, at least 6, at least 7, or at least 8 HLA alleles. Cells administered to a subject in need thereof can be haploidentical to the subject. Cells administered to a subject in need thereof can be from a donor that is related to the subject. Cells administered to a subject in need thereof can be from a donor that is not related to the subject.

In certain aspects, cryopreserved cells (e.g., engineered cells) are thawed and washed as described herein and allowed to rest for one hour at room temperature prior to activation using the methods of the present disclosure. In an aspect, a composition comprising an engineered cell can include a dosage form of a cell, e.g., a unit dosage form.

### EXAMPLES

All patents and publications mentioned in the specification are indicative of the levels of those skilled in the art to which the present disclosure pertains. The publications discussed throughout are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

In cases wherein commercially available reagents and equipment were utilized, protocols used were according to manufacturer instructions unless otherwise indicated.

### Example 1. Multicistronic Constructs

Multicistronic constructs were designed to test the effect of inserting at least one nucleic acid encoding a chimeric antigen receptor (CAR) between or adjacent to nucleic acids encoding the γδTCR receptor monomers or fragments thereof. These constructs further encoded one or more self-cleaving peptide sequences. Lentiviral vectors were generated to allow for genomic integration of DNA sequences that encode the multiple proteins in different order. The lentiviral vectors contained a tricistronic or tetracistronic sequence encoding a gamma TCR chain or fragment thereof and a delta TCR chain or fragment, a CAR and one or more 2A self-cleaving peptide sequences (e.g., P2A or T2A) connecting the three or four protein encoding sequences.

### Design of Multicistronic γ-TCR-BCMA-CAR-δ-TCR and Control Constructs

Multicistronic constructs comprising CL5_g_BCMA-BBz_d (SEQ ID NO. 111, encoded polypeptide SEQ ID NO. 112), D25_g_BCMA_BBz_d (SEQ ID NO: 173, encoded polypeptide SEQ ID NO: 174) or controls BCMA-BBz_EGFP (SEQ ID NO. 113, encoded polypeptide SEQ ID NO. 114), 41BBL_BCMA-28z (SEQ ID NO. 115 and encoded polypeptide SEQ ID NO. 116) were constructed by cloning/assembly of the sequences. Additional multicistronic constructs comprising CI5_g_BCMA-CAR-T2A_d (SEQ ID NO: 151, encoded polypeptide SEQ ID NO: 152) and CI5_g_CEA-CAR-P2A_d (SEQ ID NO: 157, encoded polypeptide SEQ ID NO: 158) and controls BCMA-CAR-P2A (SEQ ID NO: 153, encoded polypeptide SEQ ID NO: 154), BCMA-CAR-T2A (SEQ ID NO: 113, encoded polypeptide SEQ ID NO: 114) and CEA-CAR-P2A (SEQ ID NO: 155, encoded polypeptide SEQ ID NO: 156) were also prepared. Table 1 provides the sequences for some of the constructs used in the examples provided herein.

TABLE 1
Constructs and encoded polypeptide sequence

| SEQ | | |
|---|---|---|
| ID. | | |
| NOS | Name | Sequence |
| 111 | CL5_g_ | atggtgtccctgctgcacgccagcaccctggccgtgctgggcgccctgtgcgtgtatggcgccggacacctggaacagccc |
| | BCMA- | cagatcagcagcaccaagaccctgagcaagaccgccaggctggaatgcgtggtgtccggcatcaccatcagcgccacct |
| | CAR_d | ccgtgtactggtacagagagagacccggcgaggtcatccagttcctggtgtccatcagctacgacggcaccgtgcggaaa |
| | | gagagcggcatccccagcggcaagttcgaggtggacagaatccccgagaccagcacctccaccctgaccatccacaac |
| | | gtggagaagcaggacatcgccacctactactgcgccctgtgggagatccaggaactgggcaagaaaatcaaggtgttcg |
| | | gccctggcaccaagctgatcatcaccgacaagcagctggacgccgacgtgagccccaagcctaccatcttcctgcccagc |
| | | atcgccgagaccaagctgcagaaggccggcacctacctgtgcctgctggaaaagttcttccccgacgtgatcaagatccac |
| | | tgggaggaaaagaagagcaacaccatcctgggcagccaggaaggcaataccatgaaaaccaacgacacctacatga |
| | | agttcagctggctgaccgtgcccgagaagagcctggacaaagagcacagatgcatcgtccggcacgagaacaacaag |
| | | aacggcgtggaccaggaaatcatcttcccccccatcaagaccgatgtgatcacaatggaccccaaggacaactgcagca |
| | | aggacgccaacgataccctgctgctgcagctgaccaacaccagcgcctactacatgtatctcctgctgctgctgaagagcgt |
| | | ggtgtacttcgccatcatcacctgctgtctcctgaggcggaccgccttctgctgcaacggcgagaagagcggatctggcgaa |
| | | ggccgcggcagcctgctgacctgcggcgacgtggaagagaaccctgggcccatggcactccccgtcaccgcccttctcttg |
| | | cccctcgccctgctgctgcatgctgccaggcccgacattgtgctcactcagtcacctcccagcctggccatgagcctgggaa |
| | | aaagggccaccatctcctgtagagccagtgagtccgtcacaatcttggggagccatcttattcactggtatcagcagaagcc |
| | | cgggcagcctccaacccttcttattcagctcgcgtcaaacgtccagacgggtgtacctgccagattttctggtagcgggtcccg |
| | | cactgattttacactgaccatagatccagtggaagaagacgatgtggccgtgtattattgtctgcagagcagaacgattcctcg |
| | | cacatttggtgggggtactaagctggagattaagggaagcacgtccggctcagggaagccgggctccggcgagggaagc |
| | | acgaaggggcaaattcagctggtccagagcggacctgagctgaaaaaacccggcgagactgttaagatcagttgtaaag |
| | | catctggctataccttcaccgactacagcataaattgggtgaaacgggcccctggaaagggcctcaaatggatgggttggat |
| | | caataccgaaactagggagcctgcttatgcatatgacttccgcgggagattcgccttttcactcgagacatctgcctctactgct |
| | | tacctccaaataaacaacctcaagtatgaagatacagccacttacttttgcgccctcgactatagttacgcgatggactactgg |
| | | ggacagggaactagtgttaccgtcagttccacaacaacccctgcccccagacctcctaccccagcccctacaattgccagc |
| | | cagcctctgagcctgagacccgaggcttgtagaccagctgctggcggagccgtgcacaccagaggactggatttcgcctg |
| | | cgacatctacatctgggcccctctggccggcacatgtggcgtgctgctgctgagcctcgtgatcaccctgtactgcaagcggg |
| | | gcagaaagaaactgctgtacatctttaagcagcccttcatgcggcccgtgcagaccacccaggaagaggacggctgctcc |
| | | tgcagattccccgaggaagaagaaggcggctgcgagctgagagtgaagttcagcagatccgccgacgcccctgcctaca |
| | | agcagggacagaaccagctgtacaacgagctgaacctgggcagacgggaagagtacgacgtgctggacaagcggag |
| | | aggccgggaccctgaaatgggcggcaagcccagaagaaagaacccccaggaaggcctgtataacgaactgcagaaa |
| | | gacaagatggccgaggcctacagcgagatcggaatgaagggcgagcggagaagaggcaagggccacgatggcctgt |
| | | accagggcctgagcaccgccaccaaggacacctatgacgccctgcacatgcaggccctgcctccaagaggcagcggc |
| | | gccaccaatttcagcctgctgaaacaggctggagacgtggaagaaaaccctggcccgatggagcggatcagcagcctga |
| | | tccacctgagcctgttctgggccggagtgatgagcgccatcgagctggtgcccgagcaccagaccgtgcccgtgagcatcg |
| | | gcgtgcccgccaccctgcggtgcagcatgaagggcgaggccatcggcaactactacatcaactggtacagaaagaccc |
| | | agggcaacaccatgaccttcatctaccgggagaaggacatctacggccctggcttcaaggacaacttccagggcgacatc |
| | | gacatcgccaagaacctggccgtgctgaagatcctggcccccagcgagagggacgagggcagctactactgcgcctgc |
| | | gacgccctgaagagaaccgacaccgacaagctgatcttcggcaagggcacccgggtgaccgtggagcccagaagcca |
| | | gccccacaccaagcccagcgtgttcgtgatgaagaacggcaccaacgtggcctgcctggtgaaagagttctaccccaag |
| | | gacatccggatcaacctggtgtccagcaagaagatcaccgagttcgaccccgccatcgtgatcagccccagcggcaagta |
| | | caacgccgtgaagctgggcaagtacgaggacagcaacagcgtgacctgcagcgtgcagcacgacaacaagaccgtgc |
| | | acagcaccgacttcgaggtgaaaaccgactccaccgaccacgtgaagcccaaagagaccgagaacaccaagcagcc |
| | | cagcaagagctgccacaagcccaaggccatcgtgcacaccgagaaggtgaacatgatgagcctgaccgtgctgggcct |
| | | gcggatgctgttcgccaagacagtggccgtgaacttcctgctgaccgccaagctgttcttcctgtga |
| 112 | CL5_g_ | MVSLLHASTLAVLGALCVYGAGHLEQPQISSTKTLSKTARLECVVSGITISATSVYWYRE |
| | BCMA- | RPGEVIQFLVSISYDGTVRKESGIPSGKFEVDRIPETSTSTLTIHNVEKQDIATYYCALWEI |
| | CAR_d | QELGKKIKVFGPGTKLIITDKQLDADVSPKPTIFLPSIAETKLQKAGTYLCLLEKFFPDVIKI |
| | | HWEEKKSNTILGSQEGNTMKTNDTYMKFSWLTVPEKSLDKEHRCIVRHENNKNGVDQE |
| | | IIFPPIKTDVITMDPKDNCSKDANDTLLLQLTNTSAYYMYLLLLLKSVVYFAIITCCLLRRTA |
| | | FCCNGEKSGSGEGRGSLLTCGDVEENPGPMALPVTALLLPLALLLHAARPDIVLTQSPP |
| | | SLAMSLGKRATISCRASESVTILGSHLIHWYQQKPGQPPTLLIQLASNVQTGVPARFSGS |
| | | GSRTDFTLTIDPVEEDDVAVYYCLQSRTIPRTFGGGTKLEIKGSTSGSGKPGSGEGSTK |
| | | GQIQLVQSGPELKKPGETVKISCKASGYTFTDYSINWVKRAPGKGLKWMGWINTETREP |
| | | AYAYDFRGRFAFSLETSASTAYLQINNLKYEDTATYFCALDYSYAMDYWGQGTSVTVSS |
| | | TTTPAPRPPTPAPTIASQPLSLRPEACRPAAGGAVHTRGLDFACDIYIWAPLAGTCGVLL |
| | | LSLVITLYCKRGRKKLLYIFKQPFMRPVQTTQEEDGCSCRFPEEEEGGCELRVKFSRSA |
| | | DAPAYKQGQNQLYNELNLGRREEYDVLDKRRGRDPEMGGKPRRKNPQEGLYNELQKD |
| | | KMAEAYSEIGMKGERRRGKGHDGLYQGLSTATKDTYDALHMQALPPRGSGATNFSLLK |
| | | QAGDVEENPGPMERISSLIHLSLFWAGVMSAIELVPEHQTVPVSIGVPATLRCSMKGEAI |
| | | GNYYINWYRKTQGNTMTFIYREKDIYGPGFKDNFQGDIDIAKNLAVLKILAPSERDEGSY |
| | | YCACDALKRTDTDKLIFGKGTRVTVEPRSQPHTKPSVFVMKNGTNVACLVKEFYPKDIRI |
| | | NLVSSKKITEFDPAIVISPSGKYNAVKLGKYEDSNSVTCSVQHDNKTVHSTDFEVKTDST |
| | | DHVKPKETENTKQPSKSCHKPKAIVHTEKVNMMSLTVLGLRMLFAKTVAVNFLLTAKLFF |
| | | L* |
| 113 | BCMA- | atggcactccccgtcaccgcccttctcttgcccctcgccctgctgctgcatgctgccaggcccgacattgtgctcactcagtcac |
| | CAR_EGFP | ctcccagcctggccatgagcctgggaaaaagggccaccatctcctgtagagccagtgagtccgtcacaatcttggggagc |
| | | catcttattcactggtatcagcagaagcccgggcagcctccaacccttcttattcagctcgcgtcaaacgtccagacgggtgta |
| | | cctgccagattttctggtagcgggtcccgcactgattttacactgaccatagatccagtggaagaagacgatgtggccgtgtat |
| | | tattgtctgcagagcagaacgattcctcgcacatttggtgggggtactaagctggagattaagggaagcacgtccggctcag |
| | | ggaagccgggctccggcgagggaagcacgaaggggcaaattcagctggtccagagcggacctgagctgaaaaaacc |
| | | cggcgagactgttaagatcagttgtaaagcatctggctataccttcaccgactacagcataaattgggtgaaacgggcccct |
| | | ggaaagggcctcaaatggatgggttggatcaataccgaaactagggagcctgcttatgcatatgacttccgcgggagattc |
| | | gccttttcactcgagacatctgcctctactgcttacctccaaataaacaacctcaagtatgaagatacagccacttacttttgcgc |
| | | cctcgactatagttacgcgatggactactggggacagggaacctccgttaccgtcagttccacaacaacccctgcccccag |
| | | acctcctaccccagcccctacaattgccagccagcctctgagcctgaggcccgaggcttgtagaccagctgctggcggagc |
| | | cgtgcacaccagaggactggatttcgcctgcgacatctacatctgggcccctctggccggcacatgtggcgtgctgctgctga |
| | | gcctcgtgatcaccctgtactgcaagcggggcagaaagaaactgctgtacatctttaagcagcccttcatgcggcccgtgca |
| | | gaccacccaggaagaggacggctgctcctgcagattccccgaggaagaagaaggcggctgcgagctgagagtgaagtt |
| | | cagcagatccgccgacgcccctgcctacaagcagggacagaaccagctgtacaacgagctgaacctgggcagacggg |
| | | aagagtacgacgtgctggacaagcggagaggccgggaccctgaaatgggcggcaagcccagaagaaagaaccccc |
| | | aggaaggcctgtataacgaactgcagaaagacaagatggccgaggcctacagcgagatcggaatgaagggcgagcg |
| | | gagaagaggcaagggccacgatggcctgtaccagggcctgagcaccgccaccaaggacacctatgacgccctgcaca |
| | | tgcaggccctgcctccaagaggcagcggcgaaggccgcggcagcctgctgacctgcggcgatgtggaagaaaaccctg |
| | | ggcccatggtgagcaagggcgaggagctgttcaccggggtggtgcccatcctggtcgagctggacggcgacgtaaacgg |
| | | ccacaagttcagcgtgtccggcgagggcgagggcgatgccacctacggcaagctgaccctgaagttcatctgcaccaccg |
| | | gcaagctgcccgtgccctggcccaccctcgtgaccaccctgacctacggcgtgcagtgcttcagccgctaccccgaccaca |
| | | tgaagcagcacgacttcttcaagtccgccatgcccgaaggctacgtccaggagcgcaccatcttcttcaaggacgacggca |
| | | actacaagacccgcgccgaggtgaagttcgagggcgacaccctggtgaaccgcatcgagctgaagggcatcgacttcaa |
| | | ggaggacggcaacatcctggggcacaagctggagtacaactacaacagccacaacgtctatatcatggccgacaagca |
| | | gaagaacggcatcaaggtgaacttcaagatccgccacaacatcgaggacggcagcgtgcagctcgccgaccactacca |
| | | gcagaacacccccatcggcgacggccccgtgctgctgcccgacaaccactacctgagcacccagtccgccctgagcaa |
| | | agaccccaacgagaagcgcgatcacatggtcctgctggagttcgtgaccgccgccgggatcactctcggcatggacgag |
| | | ctgtacaagtaa |
| 114 | BCMA- | MALPVTALLLPLALLLHAARPDIVLTQSPPSLAMSLGKRATISCRASESVTILGSHLIHWYQ |
| | CAR_EGFP | QKPGQPPTLLIQLASNVQTGVPARFSGSGSRTDFTLTIDPVEEDDVAVYYCLQSRTIPRT |
| | (BCMA- | FGGGTKLEIKGSTSGSGKPGSGEGSTKGQIQLVQSGPELKKPGETVKISCKASGYTFTD |
| | CAR-TCA) | YSINWVKRAPGKGLKWMGWINTETREPAYAYDFRGRFAFSLETSASTAYLQINNLKYED |
| | | TATYFCALDYSYAMDYWGQGTSVTVSSTTTPAPRPPTPAPTIASQPLSLRPEACRPAAG |
| | | GAVHTRGLDFACDIYIWAPLAGTCGVLLLSLVITLYCKRGRKKLLYIFKQPFMRPVQTTQE |
| | | EDGCSCRFPEEEEGGCELRVKFSRSADAPAYKQGQNQLYNELNLGRREEYDVLDKRR |
| | | GRDPEMGGKPRRKNPQEGLYNELQKDKMAEAYSEIGMKGERRRGKGHDGLYQGLST |
| | | ATKDTYDALHMQALPPRGSGEGRGSLLTCGDVEENPGPMVSKGEELFTGVVPILVELD |
| | | GDVNGHKFSVSGEGEGDATYGKLTLKFICTTGKLPVPWPTLVTTLTYGVQCFSRYPDH |
| | | MKQHDFFKSAMPEGYVQERTIFFKDDGNYKTRAEVKFEGDTLVNRIELKGIDFKEDGNIL |
| | | GHKLEYNYNSHNVYIMADKQKNGIKVNFKIRHNIEDGSVQLADHYQQNTPIGDGPVLLPD |
| | | NHYLSTQSALSKDPNEKRDHMVLLEFVTAAGITLGMDELYK* |
| 115 | 41BBL_ | atggaatatgccagtgacgcatctctcgatcccgaagccccttggccaccggcgccaagagctagagcttgcagagtgttg |
| | BCMA-CAR | ccttgggctttggtggccggactgctgctcctgctgctgctggctgctgcctgtgccgtgtttctcgcatgtccttgggctgtgtccg |
| | | gtgcaagagcctctcctggatctgccgcgagtccgcgcctgcgagagggaccagaattgtcaccggatgaccctgcaggc |
| | | cttctcgatctcaggcaaggaatgtttgcgcagctggtggcacaaaacgtgttgcttatagacggaccacttagctggtattctg |
| | | atcccggcctggccggggtttcattgaccggcggcctttcctacaaggaagataccaaggaactggtggtcgccaaggccg |
| | | gagtatactatgttttcttccaattggagttgcggcgcgtcgtggcaggggaagggagcggttctgtctctttggctctccacctg |
| | | caacccttgagatccgcggctggagctgcagctttggcgctcactgttgacctgccacctgcaagttccgaggctcggaactc |
| | | agcgttcgggtttcaaggcagactgctgcacctgagcgccgggcaaagacttggtgtacacttgcacactgaggctagagc |
| | | ccgccacgcttggcaactcacacaaggggctacagtgcttggcctgttccgagtaacgcccgaaattccagcgggcctgcc |
| | | aagtcctagatctgaaggcagcggcgaaggccgcggcagcctgctgacctgcggagacgtggaagaaaaccctggccc |
| | | gatggcactccccgtcaccgcccttctcttgcccctcgccctgctgctgcatgctgccaggcccgacattgtgctcactcagtca |
| | | cctcccagcctggccatgagcctgggaaaaagggccaccatctcctgtagagccagtgagtccgtcacaatcttggggag |
| | | ccatcttattcactggtatcagcagaagcccgggcagcctccaacccttcttattcagctcgcgtcaaacgtccagacgggtgt |
| | | acctgccagattttctggtagcgggtcccgcactgattttacactgaccatagatccagtggaagaagacgatgtggccgtgt |
| | | attattgtctgcagagcagaacgattcctcgcacatttggtgggggtactaagctggagattaagggaagcacgtccggctca |
| | | gggaagccgggctccggcgagggaagcacgaaggggcaaattcagctggtccagagcggacctgagctgaaaaaac |
| | | ccggcgagactgttaagatcagttgtaaagcatctggctataccttcaccgactacagcataaattgggtgaaacgggcccc |
| | | tggaaagggcctcaaatggatgggttggatcaataccgaaactagggagcctgcttatgcatatgacttccgcgggagattc |
| | | gccttttcactcgagacatctgcctctactgcttacctccaaataaacaacctcaagtatgaagatacagccacttacttttgcgc |
| | | cctcgactatagttacgcgatggactactggggacagggaacCTCCGTTACCGTCAGTTCCACCACTAC |
| | | TCCAGCCCCGAGGCCCCCAACACCAGCTCCTACTATCGCATCTCAGCCACTTAGTC |
| | | TTAGACCTGAGGCATGCAGGCCCGCCGCCGGGGGTGCCGTGCATACCCGCGGACT |
| | | GGACTTTGCGTGTGACATCTATATTTGGGCCCCTCTGGCCGGTACTTGCGGTGTTCT |
| | | TTTGCTGTCACTGGTTATTACTCTCTATTGCAACCATAGAAACAGATCAAAGAGGTCC |
| | | AGACTGCTGCACTCGGACTACATGAACATGACCCCCCGCAGGCCCGGCCCCACCA |
| | | GAAAACACTATCAGCCTTACGCCCCCCCCAGGGATTTTGCTGCCTATAGATCTCGCG |
| | | TGAAGTTTAGCAGAAGCGCAGACGCTCCCGCATATCAACAGGGTCAAAATCAGCTAT |
| | | ATAATGAGCTGAATCTTGGCCGGCGAGAGGAATACGATGTACTGGACAAAAGACGG |
| | | GGGCGAGATCCCGAGATGGGAGGAAAACCTCGACGTAAAAATCCTCAAGAGGGTCT |
| | | GTATAACGAGCTGCAGAAAGACAAAATGGCGGAGGCGTACAGCGAAATAGGGATGA |
| | | AGGGCGAGCGTCGACGAGGTAAGGGACATGATGGACTGTATCAAGGACTGTCTACG |
| | | GCCACAAAGGATACATACGACGCTCTCCACATGCAGGCTCTGCCACCTCGGtga |
| 116 | 41BBL_ | MEYASDASLDPEAPWPPAPRARACRVLPWALVAGLLLLLLLAAACAVFLACPWAVSGA |
| | BCMA- | RASPGSAASPRLREGPELSPDDPAGLLDLRQGMFAQLVAQNVLLIDGPLSWYSDPGLA |
| | 28z-CAR | GVSLTGGLSYKEDTKELVVAKAGVYYVFFQLELRRVVAGEGSGSVSLALHLQPLRSAAG |
| | | AAALALTVDLPPASSEARNSAFGFQGRLLHLSAGQRLGVHLHTEARARHAWQLTQGAT |
| | | VLGLFRVTPEIPAGLPSPRSEGSGEGRGSLLTCGDVEENPGPMALPVTALLLPLALLLHA |
| | | ARPDIVLTQSPPSLAMSLGKRATISCRASESVTILGSHLIHWYQQKPGQPPTLLIQLASNV |
| | | QTGVPARFSGSGSRTDFTLTIDPVEEDDVAVYYCLQSRTIPRTFGGGTKLEIKGSTSGSG |
| | | KPGSGEGSTKGQIQLVQSGPELKKPGETVKISCKASGYTFTDYSINWVKRAPGKGLKW |
| | | MGWINTETREPAYAYDFRGRFAFSLETSASTAYLQINNLKYEDTATYFCALDYSYAMDY |
| | | WGQGTSVTVSSTTTPAPRPPTPAPTIASQPLSLRPEACRPAAGGAVHTRGLDFACDIYI |
| | | WAPLAGTCGVLLLSLVITLYCNHRNRSKRSRLLHSDYMNMTPRRPGPTRKHYQPYAPP |
| | | RDFAAYRSRVKFSRSADAPAYQQGQNQLYNELNLGRREEYDVLDKRRGRDPEMGGKP |
| | | RRKNPQEGLYNELQKDKMAEAYSEIGMKGERRRGKGHDGLYQGLSTATKDTYDALHM |
| | | QALPPR* |
| 151 | Cl5_g_ | atggtgtccctgctgcacgccagcaccctggccgtgctgggcgccctgtgcgtgtatggcgccggacacctggaacagccc |
| | BCMA-CAR- | cagatcagcagcaccaagaccctgagcaagaccgccaggctggaatgcgtggtgtccggcatcaccatcagcgccacct |
| | T2A_d | ccgtgtactggtacagagagagacccggcgaggtcatccagttcctggtgtccatcagctacgacggcaccgtgcggaaa |
| | | gagagcggcatccccagcggcaagttcgaggtggacagaatccccgagaccagcacctccaccctgaccatocacaac |
| | | gtggagaagcaggacatcgccacctactactgcgccctgtgggagatccaggaactgggcaagaaaatcaaggtgttcg |
| | | gccctggcaccaagctgatcatcaccgacaagcagctggacgccgacgtgagccccaagcctaccatcttcctgcccagc |
| | | atcgccgagaccaagctgcagaaggccggcacctacctgtgcctgctggaaaagttcttccccgacgtgatcaagatccac |
| | | tgggaggaaaagaagagcaacaccatcctgggcagccaggaaggcaataccatgaaaaccaacgacacctacatga |
| | | agttcagctggctgaccgtgcccgagaagagcctggacaaagagcacagatgcatcgtccggcacgagaacaacaag |
| | | aacggcgtggaccaggaaatcatcttcccccccatcaagaccgatgtgatcacaatggaccccaaggacaactgcagca |
| | | aggacgccaacgataccctgctgctgcagctgaccaacaccagcgcctactacatgtatctcctgctgctgctgaagagcgt |
| | | ggtgtacttcgccatcatcacctgctgtctcctgaggcggaccgccttctgctgcaacggcgagaagagcggatctggcgcc |
| | | accaatttcagcctgCtgaaacaggctggagacgtggaagagaaccctgggcccatggcactccccgtcaccgcccttct |
| | | cttgcccctcgccctgctgctgcatgctgccaggcccgacattgtgctcactcagtcacctcccagcctggccatgagcctgg |
| | | gaaaaagggccaccatctcctgtagagccagtgagtccgtcacaatcttggggagccatcttattcactggtatcagcagaa |
| | | gcccgggcagcctccaacccttcttattcagctcgcgtcaaacgtccagacgggtgtacctgccagattttctggtagcgggtc |
| | | ccgcactgattttacactgaccatagatccagtggaagaagacgatgtggccgtgtattattgtctgcagagcagaacgattc |
| | | ctcgcacatttggtgggggtactaagctggagattaagggaagcacgtccggctcagggaagccgggctccggcgaggg |
| | | aagcacgaaggggcaaattcagctggtccagagcggacctgagctgaaaaaacccggcgagactgttaagatcagttgt |
| | | aaagcatctggctataccttcaccgactacagcataaattgggtgaaacgggcccctggaaagggcctcaaatggatgggt |
| | | tggatcaataccgaaactagggagcctgcttatgcatatgacttccgcgggagattcgccttttcactcgagacatctgcctcta |
| | | ctgcttacctccaaataaacaacctcaagtatgaagatacagccacttacttttgcgccctcgactatagttacgcgatggact |
| | | actggggacagggaactagtgttaccgtcagttccacaacaacccctgcccccagacctcctaccccagcccctacaattg |
| | | ccagccagcctctgagcctgagacccgaggcttgtagaccagctgctggcggagccgtgcacaccagaggactggatttc |
| | | gcctgcgacatctacatctgggcccctctggccggcacatgtggcgtgctgctgctgagcctcgtgatcaccctgtactgcaa |
| | | gcggggcagaaagaaactgctgtacatctttaagcagcccttcatgcggcccgtgcagaccacccaggaagaggacggc |
| | | tgctcctgcagattccccgaggaagaagaaggcggctgcgagctgagagtgaagttcagcagatccgccgacgcccctg |
| | | cctacaagcagggacagaaccagctgtacaacgagctgaacctgggcagacgggaagagtacgacgtgctggacaag |
| | | cggagaggccgggaccctgaaatgggcggcaagcccagaagaaagaacccccaggaaggcctgtataacgaactgc |
| | | agaaagacaagatggccgaggcctacagcgagatcggaatgaagggcgagcggagaagaggcaagggccacgatg |
| | | gcctgtaccagggcctgagcaccgccaccaaggacacctatgacgccctgcacatgcaggccctgcctccaagaGGC |
| | | AGCGGCGAAGGCCGCggcagcctgctgacctgcggagacgtggaagaaaaccctggcccgatggagcggat |
| | | cagcagcctgatccacctgagcctgttctgggccggagtgatgagcgccatcgagctggtgcccgagcaccagaccgtgc |
| | | ccgtgagcatcggcgtgcccgccaccctgcggtgcagcatgaagggcgaggccatcggcaactactacatcaactggtac |
| | | agaaagacccagggcaacaccatgaccttcatctaccgggagaaggacatctacggccctggcttcaaggacaacttcc |
| | | agggcgacatcgacatcgccaagaacctggccgtgctgaagatcctggcccccagcgagagggacgagggcagctact |
| | | actgcgcctgcgacgccctgaagagaaccgacaccgacaagctgatcttcggcaagggcacccgggtgaccgtggagc |
| | | ccagaagccagccccacaccaagcccagcgtgttcgtgatgaagaacggcaccaacgtggcctgcctggtgaaagagtt |
| | | ctaccccaaggacatccggatcaacctggtgtccagcaagaagatcaccgagttcgaccccgccatcgtgatcagcccca |
| | | gcggcaagtacaacgccgtgaagctgggcaagtacgaggacagcaacagcgtgacctgcagcgtgcagcacgacaac |
| | | aagaccgtgcacagcaccgacttcgaggtgaaaaccgactccaccgaccacgtgaagcccaaagagaccgagaaca |
| | | ccaagcagcccagcaagagctgccacaagcccaaggccatcgtgcacaccgagaaggtgaacatgatgagcctgacc |
| | | gtgctgggcctgcggatgctgttcgccaagacagtggccgtgaacttcctgctgaccgccaagctgttcttcctg |
| 152 | Cl5_g_ | MVSLLHASTLAVLGALCVYGAGHLEQPQISSTKTLSKTARLECVVSGITISATSVYWYRE |
| | BCMA-CAR- | RPGEVIQFLVSISYDGTVRKESGIPSGKFEVDRIPETSTSTLTIHNVEKQDIATYYCALWEI |
| | T2A_d | QELGKKIKVFGPGTKLIITDKQLDADVSPKPTIFLPSIAETKLQKAGTYLCLLEKFFPDVIKI |
| | | HWEEKKSNTILGSQEGNTMKTNDTYMKFSWLTVPEKSLDKEHRCIVRHENNKNGVDQE |
| | | IIFPPIKTDVITMDPKDNCSKDANDTLLLQLTNTSAYYMYLLLLLKSVVYFAIITCCLLRRTA |
| | | FCCNGEKSGSGATNFSLLKQAGDVEENPGPMALPVTALLLPLALLLHAARPDIVLTQSPP |
| | | SLAMSLGKRATISCRASESVTILGSHLIHWYQQKPGQPPTLLIQLASNVQTGVPARFSGS |
| | | GSRTDFTLTIDPVEEDDVAVYYCLQSRTIPRTFGGGTKLEIKGSTSGSGKPGSGEGSTK |
| | | GQIQLVQSGPELKKPGETVKISCKASGYTFTDYSINWVKRAPGKGLKWMGWINTETREP |
| | | AYAYDFRGRFAFSLETSASTAYLQINNLKYEDTATYFCALDYSYAMDYWGQGTSVTVSS |
| | | TTTPAPRPPTPAPTIASQPLSLRPEACRPAAGGAVHTRGLDFACDIYIWAPLAGTCGVLL |
| | | LSLVITLYCKRGRKKLLYIFKQPFMRPVQTTQEEDGCSCRFPEEEEGGCELRVKFSRSA |
| | | DAPAYKQGQNQLYNELNLGRREEYDVLDKRRGRDPEMGGKPRRKNPQEGLYNELQKD |
| | | KMAEAYSEIGMKGERRRGKGHDGLYQGLSTATKDTYDALHMQALPPRGSGEGRGSLL |
| | | TCGDVEENPGPMERISSLIHLSLFWAGVMSAIELVPEHQTVPVSIGVPATLRCSMKGEAI |
| | | GNYYINWYRKTQGNTMTFIYREKDIYGPGFKDNFQGDIDIAKNLAVLKILAPSERDEGSY |
| | | YCACDALKRTDTDKLIFGKGTRVTVEPRSQPHTKPSVFVMKNGTNVACLVKEFYPKDIRI |
| | | NLVSSKKITEFDPAIVISPSGKYNAVKLGKYEDSNSVTCSVQHDNKTVHSTDFEVKTDST |
| | | DHVKPKETENTKQPSKSCHKPKAIVHTEKVNMMSLTVLGLRMLFAKTVAVNFLLTAKLFF |
| | | L |
| 153 | BCMA-CAR- | atggcactccccgtcaccgcccttctcttgcccctcgccctgctgctgcatgctgccaggcccgacattgtgctcactcagtcac |
| | P2A | ctcccagcctggccatgagcctgggaaaaagggccaccatctcctgtagagccagtgagtccgtcacaatcttggggagc |
| | | catcttattcactggtatcagcagaagcccgggcagcctccaacccttcttattcagctcgcgtcaaacgtccagacgggtgta |
| | | cctgccagattttctggtagcgggtcccgcactgattttacactgaccatagatccagtggaagaagacgatgtggccgtgtat |
| | | tattgtctgcagagcagaacgattcctcgcacatttggtgggggtactaagctggagattaagggaagcacgtccggctcag |
| | | ggaagccgggctccggcgagggaagcacgaaggggcaaattcagctggtccagagcggacctgagctgaaaaaacc |
| | | cggcgagactgttaagatcagttgtaaagcatctggctataccttcaccgactacagcataaattgggtgaaacgggcccct |
| | | ggaaagggcctcaaatggatgggttggatcaataccgaaactagggagcctgcttatgcatatgacttccgcgggagattc |
| | | gccttttcactcgagacatctgcctctactgcttacctccaaataaacaacctcaagtatgaagatacagccacttacttttgcgc |
| | | cctcgactatagttacgcgatggactactggggacagggaactagtgttaccgtcagttccacaacaacccctgcccccag |
| | | acctcctaccccagcccctacaattgccagccagcctctgagcctgagacccgaggcttgtagaccagctgctggcggagc |
| | | cgtgcacaccagaggactggatttcgcctgcgacatctacatctgggcccctctggccggcacatgtggcgtgctgctgctga |
| | | gcctcgtgatcaccctgtactgcaagcggggcagaaagaaactgctgtacatctttaagcagcccttcatgcggcccgtgca |
| | | gaccacccaggaagaggacggctgctcctgcagattccccgaggaagaagaaggcggctgcgagctgagagtgaagtt |
| | | cagcagatccgccgacgcccctgcctacaagcagggacagaaccagctgtacaacgagctgaacctgggcagacggg |
| | | aagagtacgacgtgctggacaagcggagaggccgggaccctgaaatgggggcaagcccagaagaaagaaccccc |
| | | aggaaggcctgtataacgaactgcagaaagacaagatggccgaggcctacagcgagatcggaatgaagggcgagcg |
| | | gagaagaggcaagggccacgatggcctgtaccagggcctgagcaccgccaccaaggacacctatgacgccctgcaca |
| | | tgcaggccctgcctccaagaggcagcggcgccaccaatttcagcctgctgaaacaggctggagacgtggaagaaaacc |
| | | ctgggcccatggtgagcaagggcgaggagctgttcaccggggtggtgcccatcctggtcgagctggacggcgacgtaaa |
| | | cggccacaagttcagcgtgtccggcgagggcgagggcgatgccacctacggcaagctgaccctgaagttcatctgcacc |
| | | accggcaagctgcccgtgccctggcccaccctcgtgaccaccctgacctacggcgtgcagtgcttcagccgctaccccgac |
| | | cacatgaagcagcacgacttcttcaagtccgccatgcccgaaggctacgtccaggagcgcaccatcttcttcaaggacgac |
| | | ggcaactacaagacccgcgccgaggtgaagttcgagggcgacaccctggtgaaccgcatcgagctgaagggcatcgac |
| | | ttcaaggaggacggcaacatcctggggcacaagctggagtacaactacaacagccacaacgtctatatcatggccgaca |
| | | agcagaagaacggcatcaaggtgaacttcaagatccgccacaacatcgaggacggcagcgtgcagctcgccgaccact |
| | | accagcagaacacccccatcggcgacggccccgtgctgctgcccgacaaccactacctgagcacccagtccgccctga |
| | | gcaaagaccccaacgagaagcgcgatcacatggtcctgctggagttcgtgaccgccgccgggatcactctcggcatgga |
| | | cgagctgtacaag |
| 154 | BCMA-CAR- | MALPVTALLLPLALLLHAARPDIVLTQSPPSLAMSLGKRATISCRASESVTILGSHLIHWYQ |
| | P2A | QKPGQPPTLLIQLASNVQTGVPARFSGSGSRTDFTLTIDPVEEDDVAVYYCLQSRTIPRT |
| | | FGGGTKLEIKGSTSGSGKPGSGEGSTKGQIQLVQSGPELKKPGETVKISCKASGYTFTD |
| | | YSINWVKRAPGKGLKWMGWINTETREPAYAYDFRGRFAFSLETSASTAYLQINNLKYED |
| | | TATYFCALDYSYAMDYWGQGTSVTVSSTTTPAPRPPTPAPTIASQPLSLRPEACRPAAG |
| | | GAVHTRGLDFACDIYIWAPLAGTCGVLLLSLVITLYCKRGRKKLLYIFKQPFMRPVQTTQE |
| | | EDGCSCRFPEEEEGGCELRVKFSRSADAPAYKQGQNQLYNELNLGRREEYDVLDKRR |
| | | GRDPEMGGKPRRKNPQEGLYNELQKDKMAEAYSEIGMKGERRRGKGHDGLYQGLST |
| | | ATKDTYDALHMQALPPRGSGATNFSLLKQAGDVEENPGPMVSKGEELFTGVVPILVELD |
| | | GDVNGHKFSVSGEGEGDATYGKLTLKFICTTGKLPVPWPTLVTTLTYGVQCFSRYPDH |
| | | MKQHDFFKSAMPEGYVQERTIFFKDDGNYKTRAEVKFEGDTLVNRIELKGIDFKEDGNIL |
| | | GHKLEYNYNSHNVYIMADKQKNGIKVNFKIRHNIEDGSVQLADHYQQNTPIGDGPVLLPD |
| | | NHYLSTQSALSKDPNEKRDHMVLLEFVTAAGITLGMDELYK |
| 155 | CEA-CAR- | ATGGATATGCGGGTACCAGCGCAACTGCTTGGGCTGCTTCTTTTGTGGCTGAGAGG |
| | P2A | TGCTCGGTGTGACATTCAATTGACCCAGTCTCCTTCTTCCTTGAGCGCTAGCGTCGG |
| | | CGACCGCGTCACCATCACATGCAAAGCCTCCCAGGATGTGGGAACGTCAGTAGCCT |
| | | GGTACCAGCAAAAGCCTGGAAAAGCACCTAAGCTTTTGATCTATTGGACCTCTACTA |
| | | GGCATACAGGGGTCCCGAGTCGCTTTTCCGGCTCCGGTAGCGGCACAGATTTCACC |
| | | TTCACAATATCTTCTTTACAACCAGAAGACATCGCCACTTACTACTGTCAGCAGTATT |
| | | CTTTGTACCGTTCCTTTGGGCAAGGAACAAAAGTAGAAATAAAGAGAGGTGGCTCTG |
| | | GAAGCGGCGGGTCCGGGAGTGGTGGGTCTGGTTCTGAGGTGCAGCTCGTGGAGTC |
| | | AGGCGGCGGGGTCGTCCAGCCAGGGAGATCTCTGAGGCTGAGCTGCAGCGCGTC |
| | | GGGGTTTGACTTTACCACCTATTGGATGAGTTGGGTGCGCCAGGCCCCTGGGAAGG |
| | | GACTTGAATGGATAGGCGAGATCCATCCCGACAGCTCCACAATCAATTACGCACCTT |
| | | CCTTGAAAGACAGGTTTACAATCTCGAGAGATAACGCAAAGAACACACTGTTTTTGC |
| | | AAATGGACTCACTCCGCCCTGAGGACACCGGAGTGTACTTCTGCGCGAGCCTATAT |
| | | TTTGGGTTCCCCTGGTTCGCATACTGGGGCCAGGGAACCCCCGTAACTGTCTCAAG |
| | | CGCAAAGCCaACCACTACCccagcgccgcgaccaccaacaccggcgcccACCatcgcgtcgcagcccC |
| | | TGTCTTTGCGACCTGAGGCTGCTAGGCCTGCCGCTGGCGGAGCTGTGCACACCAG |
| | | GGGGTTGGATTTCGCCCCTCGGAAGATTGAAGTTATGTATCCCCCACCCTACCTCGA |
| | | CAACGAAAAATCCAACGGGACAATAATTCACGTAAAGGGCAAACACCTGTGTCCCAG |
| | | TCCATTATTCCCCGGGCCCAGCAAACCCTTCTGGGTGCTGGTGGTGGTCGGTGGGG |
| | | TGCTGGCCTGTTATTCCTTGCTAGTTACCGTCGCCTTCATTATATTCTGGGTAAGATC |
| | | TAAGCGTTCACGCCTTCTCCACTCTGATTACATGAACATGACCCCAAGAAGGCCCGG |
| | | GCCTACCAGGAAGCACTATCAGCCGTATGCCCCACCTCGCGACTTCGCTGCCTACA |
| | | GGTCTAGGGTAAAATTCTCTCGCAGTGCAGAGCCTCCCGCTTATCAGCAGGGGCAA |
| | | AATCAATTGTACAATGAGCTGAACCTCGGACGGCGCGAAGAGTACGATGTGCTGGA |
| | | TAAGCGTCGCGGGAGAGACCCCGAGATGGGAGGCAAACCCCGCAGAAAGAATCCC |
| | | CAGGAGGGACTCTATAACGAGTTGCAGAAGGACAAAATGGCCGAGGCCTACTCGGA |
| | | AATCGGAATGAAAGGGGAAAGAAGGCGCGGTAAAGGCCACGACGGTCTCTATCAG |
| | | GGCTTATCTACAGCTACAAAGGATACTTACGATGCGCTGCACATGCAGGCACTGCC |
| | | CCCTCGGggatctggcgccaccaatttcagcctgctgaaacaggctggcgacgtggaagagaaccctgggcccAT |
| | | GCCACCGCCTAGGCTGCTCTTCTTTCTCTTGTTCCTGACACCTATGGAGGTCAGACC |
| | | CGAAGAGCCTTTGGTGGTTAAAGTGGAGGAGGGAGACAACGCCGTGCTGCAGTGTT |
| | | TGAAAGGAACATCCGACGGGCCTACCCAGCAACTGACTTGGTCCAGAGAGTCTCCC |
| | | CTGAAACCATTCCTGAAACTAAGCCTCGGGCTTCCTGGTCTGGGCATCCATATGCGA |
| | | CCTTTAGCCATCTGGCTCTTTATTTTCAATGTCTCACAGCAGATGGGAGGCTTTTACC |
| | | TGTGCCAGCCAGGCCCACCAAGTGAAAAAGCCTGGCAGCCAGGGTGGACCGTTAAT |
| | | GTTGAGGGGTCTGGCGAGCTTTTCCGGTGGAACGTCTCTGACCTCGGGGGCTTGG |
| | | GGTGCGGTCTGAAGAACCGATCGTCTGAGGGACCTTCTTCACCCAGCGGCAAGTTG |
| | | ATGAGCCCCAAGTTGTATGTCTGGGCTAAGGACCGACCGGAAATCTGGGAGGGAGA |
| | | GCCTCCCTGCCTTCCCCCTAGGGATAGCCTTAACCAGAGCCTCTCCCAAGACTTGA |
| | | CAATGGCACCAGGCAGCACCTTATGGCTCTCATGCGGGGTACCACCAGACTCAGTG |
| | | TCACGAGGACCACTGAGCTGGACTCATGTGCACCCAAAGGGCCCCAAATCTCTGCT |
| | | GAGTCTGGAACTGAAAGACGACCGACCTGCTAGGGATATGTGGGTGATGGAAACCG |
| | | GCCTGCTTCTCCCACGCGCCACAGCTCAAGATGCAGGTAAATATTATTGTCACAGAG |
| | | GAAATCTCACAATGTCCTTTCATCTGGAGATTACCGCCCGCCCCGTGCTGTGGCACT |
| | | GGCTGCTGCGTACCGGAGGGTGGAAAGTGAGCGCAGTGACCTTGGCCTATCTGAT |
| | | CTTTTGTCTGTGTAGCTTGGTGGGGATTCTGCATCTCCAGCGCGCATTGGTGCTACG |
| | | TAGGAAACGGAAAAGGATGACAGATCCCACCCGCCGGTTC |
| 156 | CEA-CAR- | MDMRVPAQLLGLLLLWLRGARCDIQLTQSPSSLSASVGDRVTITCKASQDVGTSVAWY |
| | P2A | QQKPGKAPKLLIYWTSTRHTGVPSRFSGSGSGTDFTFTISSLQPEDIATYYCQQYSLYRS |
| | | FGQGTKVEIKRGGSGSGGSGSGGSGSEVQLVESGGGVVQPGRSLRLSCSASGFDFTT |
| | | YWMSWVRQAPGKGLEWIGEIHPDSSTINYAPSLKDRFTISRDNAKNTLFLQMDSLRPED |
| | | TGVYFCASLYFGFPWFAYWGQGTPVTVSSAKPTTTPAPRPPTPAPTIASQPLSLRPEAA |
| | | RPAAGGAVHTRGLDFAPRKIEVMYPPPYLDNEKSNGTIIHVKGKHLCPSPLFPGPSKPF |
| | | WVLVVVGGVLACYSLLVTVAFIIFWVRSKRSRLLHSDYMNMTPRRPGPTRKHYQPYAPP |
| | | RDFAAYRSRVKFSRSAEPPAYQQGQNQLYNELNLGRREEYDVLDKRRGRDPEMGGKP |
| | | RRKNPQEGLYNELQKDKMAEAYSEIGMKGERRRGKGHDGLYQGLSTATKDTYDALHM |
| | | QALPPRGSGATNFSLLKQAGDVEENPGPMPPPRLLFFLLFLTPMEVRPEEPLVVKVEEG |
| | | DNAVLQCLKGTSDGPTQQLTWSRESPLKPFLKLSLGLPGLGIHMRPLAIWLFIFNVSQQ |
| | | MGGFYLCQPGPPSEKAWQPGWTVNVEGSGELFRWNVSDLGGLGCGLKNRSSEGPSS |
| | | PSGKLMSPKLYVWAKDRPEIWEGEPPCLPPRDSLNQSLSQDLTMAPGSTLWLSCGVPP |
| | | DSVSRGPLSWTHVHPKGPKSLLSLELKDDRPARDMWVMETGLLLPRATAQDAGKYYC |
| | | HRGNLTMSFHLEITARPVLWHWLLRTGGWKVSAVTLAYLIFCLCSLVGILHLQRALVLRR |
| | | KRKRMTDPTRRF |
| 157 | Cl5_g_ | atggtgtccctgctgcacgccagcaccctggccgtgctgggcgccctgtgcgtgtatggcgccggacacctggaacagccc |
| | CEA-CAR- | cagatcagcagcaccaagaccctgagcaagaccgccaggctggaatgcgtggtgtccggcatcaccatcagcgccacct |
| | P2A_d | ccgtgtactggtacagagagagacccggcgaggtcatccagttcctggtgtccatcagctacgacggcaccgtgcggaaa |
| | | gagagcggcatccccagcggcaagttcgaggtggacagaatccccgagaccagcacctccaccctgaccatccacaac |
| | | gtggagaagcaggacatcgccacctactactgcgccctgtgggagatccaggaactgggcaagaaaatcaaggtgttcg |
| | | gccctggcaccaagctgatcatcaccgacaagcagctggacgccgacgtgagccccaagcctaccatcttcctgcccagc |
| | | atcgccgagaccaagctgcagaaggccggcacctacctgtgcctgctggaaaagttcttccccgacgtgatcaagatccac |
| | | tgggaggaaaagaagagcaacaccatcctgggcagccaggaaggcaataccatgaaaaccaacgacacctacatga |
| | | agttcagctggctgaccgtgcccgagaagagcctggacaaagagcacagatgcatcgtccggcacgagaacaacaag |
| | | aacggcgtggaccaggaaatcatcttcccccccatcaagaccgatgtgatcacaatggaccccaaggacaactgcagca |
| | | aggacgccaacgataccctgctgctgcagctgaccaacaccagcgcctactacatgtatctcctgctgctgctgaagagcgt |
| | | ggtgtacttcgccatcatcacctgctgtctgctgcggcggaccgccttctgctgcaacggcgagaagagcggcagcggcga |
| | | aggccgcggcagcctgctgacctgcggcgatgtggaagaaaaccctggcccgATGGATATGCGGGTACCA |
| | | GCGCAACTGCTTGGGCTGCTTCTTTTGTGGCTGAGAGGTGCTCGGTGTGACATTCA |
| | | ATTGACCCAGTCTCCTTCTTCCTTGAGCGCTAGCGTCGGCGACCGCGTCACCATCA |
| | | CATGCAAAGCCTCCCAGGATGTGGGAACGTCAGTAGCCTGGTACCAGCAAAAGCCT |
| | | GGAAAAGCACCTAAGCTTTTGATCTATTGGACCTCTACTAGGCATACAGGGGTCCCG |
| | | AGTCGCTTTTCCGGCTCCGGTAGCGGCACAGATTTCACCTTCACAATATCTTCTTTA |
| | | CAACCAGAAGACATCGCCACTTACTACTGTCAGCAGTATTCTTTGTACCGTTCCTTTG |
| | | GGCAAGGAACAAAAGTAGAAATAAAGAGAGGTGGCTCTGGAAGCGGCGGGTCCGG |
| | | GAGTGGTGGGTCTGGTTCTGAGGTGCAGCTCGTGGAGTCAGGCGGCGGGGTCGTC |
| | | CAGCCAGGGAGATCTCTGAGGCTGAGCTGCAGCGCGTCGGGGTTTGACTTTACCAC |
| | | CTATTGGATGAGTTGGGTGCGCCAGGCCCCTGGGAAGGGACTTGAATGGATAGGC |
| | | GAGATCCATCCCGACAGCTCCACAATCAATTACGCACCTTCCTTGAAAGACAGGTTT |
| | | ACAATCTCGAGAGATAACGCAAAGAACACACTGTTTTTGCAAATGGACTCACTCCGC |
| | | CCTGAGGACACCGGAGTGTACTTCTGCGCGAGCCTATATTTTGGGTTCCCCTGGTT |
| | | CGCATACTGGGGCCAGGGAACCCCCGTAACTGTCTCAAGCGCAAAGCCaACCACTA |
| | | CCccagcgccgcgaccaccaacaccggcgcccACCatcgcgtcgcagcccCTGTCTTTGCGACCTGAG |
| | | GCTGCTAGGCCTGCCGCTGGCGGAGCTGTGCACACCAGGGGGTTGGATTTCGCCC |
| | | CTCGGAAGATTGAAGTTATGTATCCCCCACCCTACCTCGACAACGAAAAATCCAACG |
| | | GGACAATAATTCACGTAAAGGGCAAACACCTGTGTCCCAGTCCATTATTCCCCGGGC |
| | | CCAGCAAACCCTTCTGGGTGCTGGTGGTGGTCGGTGGGGTGCTGGCCTGTTATTCC |
| | | TTGCTAGTTACCGTCGCCTTCATTATATTCTGGGTAAGATCTAAGCGTTCACGCCTTC |
| | | TCCACTCTGATTACATGAACATGACCCCAAGAAGGCCCGGGCCTACCAGGAAGCAC |
| | | TATCAGCCGTATGCCCCACCTCGCGACTTCGCTGCCTACAGGTCTAGGGTAAAATTC |
| | | TCTCGCAGTGCAGAGCCTCCCGCTTATCAGCAGGGGCAAAATCAATTGTACAATGA |
| | | GCTGAACCTCGGACGGCGCGAAGAGTACGATGTGCTGGATAAGCGTCGCGGGAGA |
| | | GACCCCGAGATGGGAGGCAAACCCCGCAGAAAGAATCCCCAGGAGGGACTCTATA |
| | | ACGAGTTGCAGAAGGACAAAATGGCCGAGGCCTACTCGGAAATCGGAATGAAAGGG |
| | | GAAAGAAGGCGCGGTAAAGGCCACGACGGTCTCTATCAGGGCTTATCTACAGCTAC |
| | | AAAGGATACTTACGATGCGCTGCACATGCAGGCACTGCCCCCTCGGggatctggcgccac |
| | | caatttcagcctgctgaaacaggctggcgacgtggaagagaaccctggacctatggagcggatcagcagcctgatccacc |
| | | tgagcctgttctgggccggagtgatgagcgccatcgagctggtgcccgagcaccagaccgtgcccgtgagcatcggcgtg |
| | | cccgccaccctgcggtgcagcatgaagggcgaggccatcggcaactactacatcaactggtacagaaagacccagggc |
| | | aacaccatgaccttcatctaccgggagaaggacatctacggccctggcttcaaggacaacttccagggcgacatcgacat |
| | | cgccaagaacctggccgtgctgaagatcctggcccccagcgagagggacgagggcagctactactgcgcctgcgacgc |
| | | cctgaagagaaccgacaccgacaagctgatcttcggcaagggcacccgggtgaccgtggagcccagaagccagcccc |
| | | acaccaagcccagcgtgttcgtgatgaagaacggcaccaacgtggcctgcctggtgaaagagttctaccccaaggacatc |
| | | cggatcaacctggtgtccagcaagaagatcaccgagttcgaccccgccatcgtgatcagccccagcggcaagtacaacg |
| | | ccgtgaagctgggcaagtacgaggacagcaacagcgtgacctgcagcgtgcagcacgacaacaagaccgtgcacagc |
| | | accgacttcgaggtgaaaaccgactccaccgaccacgtgaagcccaaagagaccgagaacaccaagcagcccagca |
| | | agagctgccacaagcccaaggccatcgtgcacaccgagaaggtgaacatgatgagcctgaccgtgctgggcctgcggat |
| | | gctgttcgccaagacagtggccgtgaacttcctgctgaccgccaagctgttcttcctg |
| 158 | Cl5_g_ | MVSLLHASTLAVLGALCVYGAGHLEQPQISSTKTLSKTARLECVVSGITISATSVYWYRE |
| | CEA-CAR- | RPGEVIQFLVSISYDGTVRKESGIPSGKFEVDRIPETSTSTLTIHNVEKQDIATYYCALWEI |
| | P2A_d | QELGKKIKVFGPGTKLIITDKQLDADVSPKPTIFLPSIAETKLQKAGTYLCLLEKFFPDVIKI |
| | | HWEEKKSNTILGSQEGNTMKTNDTYMKFSWLTVPEKSLDKEHRCIVRHENNKNGVDQE |
| | | IIFPPIKTDVITMDPKDNCSKDANDTLLLQLTNTSAYYMYLLLLLKSVVYFAIITCCLLRRTA |
| | | FCCNGEKSGSGEGRGSLLTCGDVEENPGPMDMRVPAQLLGLLLLWLRGARCDIQLTQS |
| | | PSSLSASVGDRVTITCKASQDVGTSVAWYQQKPGKAPKLLIYWTSTRHTGVPSRFSGS |
| | | GSGTDFTFTISSLQPEDIATYYCQQYSLYRSFGQGTKVEIKRGGSGSGGSGSGGSGSEV |
| | | QLVESGGGVVQPGRSLRLSCSASGFDFTTYWMSWVRQAPGKGLEWIGEIHPDSSTINY |
| | | APSLKDRFTISRDNAKNTLFLQMDSLRPEDTGVYFCASLYFGFPWFAYWGQGTPVTVS |
| | | SAKPTTTPAPRPPTPAPTIASQPLSLRPEAARPAAGGAVHTRGLDFAPRKIEVMYPPPYL |
| | | DNEKSNGTIIHVKGKHLCPSPLFPGPSKPFWVLVVVGGVLACYSLLVTVAFIIFWVRSKR |
| | | SRLLHSDYMNMTPRRPGPTRKHYQPYAPPRDFAAYRSRVKFSRSAEPPAYQQGQNQL |
| | | YNELNLGRREEYDVLDKRRGRDPEMGGKPRRKNPQEGLYNELQKDKMAEAYSEIGMK |
| | | GERRRGKGHDGLYQGLSTATKDTYDALHMQALPPRGSGATNFSLLKQAGDVEENPGP |
| | | MERISSLIHLSLFWAGVMSAIELVPEHQTVPVSIGVPATLRCSMKGEAIGNYYINWYRKT |
| | | QGNTMTFIYREKDIYGPGFKDNFQGDIDIAKNLAVLKILAPSERDEGSYYCACDALKRTDT |
| | | DKLIFGKGTRVTVEPRSQPHTKPSVFVMKNGTNVACLVKEFYPKDIRINLVSSKKITEFDP |
| | | AIVISPSGKYNAVKLGKYEDSNSVTCSVQHDNKTVHSTDFEVKTDSTDHVKPKETENTK |
| | | QPSKSCHKPKAIVHTEKVNMMSLTVLGLRMLFAKTVAVNFLLTAKLFFL |
| 159 | BCMA-CAR | atggcactccccgtcaccgcccttctcttgcccctcgccctgctgctgcatgctgccaggcccgacattgtgctcactcagtcac |
| | | ctcccagcctggccatgagcctgggaaaaagggccaccatctcctgtagagccagtgagtccgtcacaatcttggggagc |
| | | catcttattcactggtatcagcagaagcccgggcagcctccaacccttcttattcagctcgcgtcaaacgtccagacgggtgta |
| | | cctgccagattttctggtagcgggtcccgcactgattttacactgaccatagatccagtggaagaagacgatgtggccgtgtat |
| | | tattgtctgcagagcagaacgattcctcgcacatttggtgggggtactaagctggagattaagggaagcacgtccggctcag |
| | | ggaagccgggctccggcgagggaagcacgaaggggcaaattcagctggtccagagcggacctgagctgaaaaaacc |
| | | cggcgagactgttaagatcagttgtaaagcatctggctataccttcaccgactacagcataaattgggtgaaacgggcccct |
| | | ggaaagggcctcaaatggatgggttggatcaataccgaaactagggagcctgcttatgcatatgacttccgcgggagattc |
| | | gccttttcactcgagacatctgcctctactgcttacctccaaataaacaacctcaagtatgaagatacagccacttacttttgcgc |
| | | cctcgactatagttacgcgatggactactggggacagggaacctccgttaccgtcagttccacaacaacccctgcccccag |
| | | acctcctaccccagcccctacaattgccagccagcctctgagcctgaggcccgaggcttgtagaccagctgctggcggagc |
| | | cgtgcacaccagaggactggatttcgcctgcgacatctacatctgggcccctctggccggcacatgtggcgtgctgctgctga |
| | | gcctcgtgatcaccctgtactgcaagcggggcagaaagaaactgctgtacatctttaagcagcccttcatgcggcccgtgca |
| | | gaccacccaggaagaggacggctgctcctgcagattccccgaggaagaagaaggcggctgcgagctgagagtgaagtt |
| | | cagcagatccgccgacgcccctgcctacaagcagggacagaaccagctgtacaacgagctgaacctgggcagacggg |
| | | aagagtacgacgtgctggacaagcggagaggccgggaccctgaaatgggggcaagcccagaagaaagaaccccc |
| | | aggaaggcctgtataacgaactgcagaaagacaagatggccgaggcctacagcgagatcggaatgaagggcgagcg |
| | | gagaagaggcaagggccacgatggcctgtaccagggcctgagcaccgccaccaaggacacctatgacgccctgcaca |
| | | tgcaggccctgcctccaaga |
| 160 | BCMA-CAR | MALPVTALLLPLALLLHAARPDIVLTQSPPSLAMSLGKRATISCRASESVTILGSHLIHWYQ |
| | | QKPGQPPTLLIQLASNVQTGVPARFSGSGSRTDFTLTIDPVEEDDVAVYYCLQSRTIPRT |
| | | FGGGTKLEIKGSTSGSGKPGSGEGSTKGQIQLVQSGPELKKPGETVKISCKASGYTFTD |
| | | YSINWVKRAPGKGLKWMGWINTETREPAYAYDFRGRFAFSLETSASTAYLQINNLKYED |
| | | TATYFCALDYSYAMDYWGQGTSVTVSSTTTPAPRPPTPAPTIASQPLSLRPEACRPAAG |
| | | GAVHTRGLDFACDIYIWAPLAGTCGVLLLSLVITLYCKRGRKKLLYIFKQPFMRPVQTTQE |
| | | EDGCSCRFPEEEEGGCELRVKFSRSADAPAYKQGQNQLYNELNLGRREEYDVLDKRR |
| | | GRDPEMGGKPRRKNPQEGLYNELQKDKMAEAYSEIGMKGERRRGKGHDGLYQGLST |
| | | ATKDTYDALHMQALPPR |
| 31 | CEA-CAR | MDMRVPAQLLGLLLLWLRGARCDIQLTQSPSSLSASVGDRVTITCKASQDVGTSVAWY |
| | | QQKPGKAPKLLIYWTSTRHTGVPSRFSGSGSGTDFTFTISSLQPEDIATYYCQQYSLYRS |
| | | FGQGTKVEIKRGGSGSGGSGSGGSGSEVQLVESGGGVVQPGRSLRLSCSASGFDFTT |
| | | YWMSWVRQAPGKGLEWIGEIHPDSSTINYAPSLKDRFTISRDNAKNTLFLQMDSLRPED |
| | | TGVYFCASLYFGFPWFAYWGQGTPVTVSSAKPTTTPAPRPPTPAPTIASQPLSLRPEAA |
| | | RPAAGGAVHTRGLDFAPRKIEVMYPPPYLDNEKSNGTIIHVKGKHLCPSPLFPGPSKPF |
| | | WVLVVVGGVLACYSLLVTVAFIIFWVRSKRSRLLHSDYMNMTPRRPGPTRKHYQPYAPP |
| | | RDFAAYRSRVKFSRSAEPPAYQQGQNQLYNELNLGRREEYDVLDKRRGRDPEMGGKP |
| | | RRKNPQEGLYNELQKDKMAEAYSEIGMKGERRRGKGHDGLYQGLSTATKDTYDALHM |
| | | QALPPR |
| 173 | D25_g_ | atggagtgggccctggccgtgctgctggcctttctgagccctgccagccagaagtccagcaacctggaaggccggaccaa |
| | BCMA_ | gagcgtgatccggcagacaggcagcagcgccgagatcacctgtgacctggccgagggcagcaccggctacatccactg |
| | BBz_d | gtatctgcaccaagaaggcaaggccccccagcggctgctgtactacgacagctacaccagctccgtggtgctggaaagc |
| | | ggcatcagccccgggaagtacgacacctacggcagcacccggaagaacctgcggatgatcctgcggaacctgatcgag |
| | | aacgacagcggcgtgtactactgcgccacctgggacggcttctactacaagaagctgttcggcagcggcaccaccctggt |
| | | ggtgacagacaagcagctggacgccgacgtgtcccccaagcctaccatcttcctgccctcaattgctgagacaaagctgca |
| | | gaaggccggcacctacctgtgcctgctggaaaagttcttcccagacgtgatcaagatccactgggaggaaaagaagtcca |
| | | acaccatcctgggcagccaagaaggcaacaccatgaagaccaacgacacctacatgaagttcagctggctgaccgtgc |
| | | ccgagaagtccctggacaaagaacaccggtgcatcgtgcggcacgagaacaacaagaacggcgtggaccaagaaat |
| | | catcttcccacccatcaagaccgacgtgatcacaatggaccccaaggacaactgcagcaaggacgccaacgataccctg |
| | | ctgctgcagctgacaaacaccagcgcctactacatgtacttgttgctgctgctgaagtccgtggtgtacttcgccatcatcacct |
| | | gctgtctcctgaggcggaccgccttctgctgcaacggcgagaagagcggatctggcgaaggccgcggcagcctgctgac |
| | | ctgcggcgacgtggaagagaaccctgggcccatggcactccccgtcaccgcccttctcttgcccctcgccctgctgctgcat |
| | | gctgccaggcccgacattgtgctcactcagtcacctcccagcctggccatgagcctgggaaaaagggccaccatctcctgt |
| | | agagccagtgagtccgtcacaatcttggggagccatcttattcactggtatcagcagaagcccgggcagcctccaacccttc |
| | | ttattcagctcgcgtcaaacgtccagacgggtgtacctgccagattttctggtagcgggtcccgcactgattttacactgaccat |
| | | agatccagtggaagaagacgatgtggccgtgtattattgtctgcagagcagaacgattcctcgcacatttggtgggggtacta |
| | | agctggagattaagggaagcacgtccggctcagggaagccgggctccggcgagggaagcacgaaggggcaaattcag |
| | | ctggtccagagcggacctgagctgaaaaaacccggcgagactgttaagatcagttgtaaagcatctggctataccttcacc |
| | | gactacagcataaattgggtgaaacgggcccctggaaagggcctcaaatggatgggttggatcaataccgaaactaggg |
| | | agcctgcttatgcatatgacttccgcgggagattcgccttttcactcgagacatctgcctctactgcttacctccaaataaacaac |
| | | ctcaagtatgaagatacagccacttacttttgcgccctcgactatagttacgcgatggactactggggacagggaactagtgtt |
| | | accgtcagttccacaacaacccctgcccccagacctcctaccccagcccctacaattgccagccagcctctgagcctgaga |
| | | cccgaggcttgtagaccagctgctggcggagccgtgcacaccagaggactggatttcgcctgcgacatctacatctgggcc |
| | | cctctggccggcacatgtggcgtgctgctgctgagcctcgtgatcaccctgtactgcaagcggggcagaaagaaactgctgt |
| | | acatctttaagcagcccttcatgcggcccgtgcagaccacccaggaagaggacggctgctcctgcagattccccgaggaa |
| | | gaagaaggcggctgcgagctgagagtgaagttcagcagatccgccgacgcccctgcctacaagcagggacagaacca |
| | | gctgtacaacgagctgaacctgggcagacgggaagagtacgacgtgctggacaagcggagaggccgggaccctgaaa |
| | | tgggcggcaagcccagaagaaagaacccccaggaaggcctgtataacgaactgcagaaagacaagatggccgaggc |
| | | ctacagcgagatcggaatgaagggcgagcggagaagaggcaagggccacgatggcctgtaccagggcctgagcacc |
| | | gccaccaaggacacctatgacgccctgcacatgcaggccctgcctccaagaggcagcggcgccaccaatttcagcctgct |
| | | gaaacaggctggagacgtggaagaaaaccctggcccgatggccatgctgctgggagccagcgtgctgatcctgtggctg |
| | | cagcccgattgggtcaacagccagcagaagaacgacgaccagcaagtgaagcagaacagccccagcctgagcgtgc |
| | | aggaaggccggatcagcatcctgaactgcgactacaccaactctatgttcgactacttcctgtggtacaagaagtaccccgc |
| | | cgagggccccaccttcctgatctccatcagcagcatcaaggacaagaacgaggacggccggttcaccgtgtttctgaaca |
| | | agagcgccaagcacctgagcctgcacatcgtgcctagccagcctggcgatagcgccgtgtacttttgtgccgccagcagcc |
| | | ccatcagaggctacaccggcagcgacaagctgatcttcggcaagggcaccagagtgaccgtggaacccagaagccag |
| | | ccccacaccaagcccagcgtgttcgtgatgaagaacggcaccaacgtggcctgcctcgtgaaagagttctaccccaagga |
| | | catccggatcaacctggtgtccagcaagaagatcaccgagttcgaccccgccatcgtgatcagcccctccggcaagtaca |
| | | acgccgtgaagctggggaagtacgaggacagcaacagcgtgacctgctccgtgcagcacgacaacaagaccgtgcac |
| | | agcaccgatttcgaagtgaaaaccgactccaccgaccacgtgaagcccaaagagacagagaacaccaagcagccca |
| | | gcaagagctgccacaagcccaaggccatcgtgcacaccgagaaagtgaacatgatgagcctgaccgtgctgggcctgc |
| | | ggatgctgttcgccaaaaccgtggccgtgaacttcctgctgaccgccaagctgttctttctg |
| 174 | D25_g_ | MEWALAVLLAFLSPASQKSSNLEGRTKSVIRQTGSSAEITCDLAEGSTGYIHWYLHQEG |
| | BCMA_ | KAPQRLLYYDSYTSSVVLESGISPGKYDTYGSTRKNLRMILRNLIENDSGVYYCATWDGF |
| | BBz_d | YYKKLFGSGTTLVVTDKQLDADVSPKPTIFLPSIAETKLQKAGTYLCLLEKFFPDVIKIHWE |
| | | EKKSNTILGSQEGNTMKTNDTYMKFSWLTVPEKSLDKEHRCIVRHENNKNGVDQEIIFPP |
| | | IKTDVITMDPKDNCSKDANDTLLLQLTNTSAYYMYLLLLLKSVVYFAIITCCLLRRTAFCCN |
| | | GEKSGSGEGRGSLLTCGDVEENPGPMALPVTALLLPLALLLHAARPDIVLTQSPPSLAM |
| | | SLGKRATISCRASESVTILGSHLIHWYQQKPGQPPTLLIQLASNVQTGVPARFSGSGSRT |
| | | DFTLTIDPVEEDDVAVYYCLQSRTIPRTFGGGTKLEIKGSTSGSGKPGSGEGSTKGQIQL |
| | | VQSGPELKKPGETVKISCKASGYTFTDYSINWVKRAPGKGLKWMGWINTETREPAYAY |
| | | DFRGRFAFSLETSASTAYLQINNLKYEDTATYFCALDYSYAMDYWGQGTSVTVSSTTTP |
| | | APRPPTPAPTIASQPLSLRPEACRPAAGGAVHTRGLDFACDIYIWAPLAGTCGVLLLSLVI |
| | | TLYCKRGRKKLLYIFKQPFMRPVQTTQEEDGCSCRFPEEEEGGCELRVKFSRSADAPA |
| | | YKQGQNQLYNELNLGRREEYDVLDKRRGRDPEMGGKPRRKNPQEGLYNELQKDKMA |
| | | EAYSEIGMKGERRRGKGHDGLYQGLSTATKDTYDALHMQALPPRGSGATNFSLLKQAG |
| | | DVEENPGPMAMLLGASVLILWLQPDWVNSQQKNDDQQVKQNSPSLSVQEGRISILNCD |
| | | YTNSMFDYFLWYKKYPAEGPTFLISISSIKDKNEDGRFTVFLNKSAKHLSLHIVPSQPGDS |
| | | AVYFCAASSPIRGYTGSDKLIFGKGTRVTVEPRSQPHTKPSVFVMKNGTNVACLVKEFY |
| | | PKDIRINLVSSKKITEFDPAIVISPSGKYNAVKLGKYEDSNSVTCSVQHDNKTVHSTDFEV |
| | | KTDSTDHVKPKETENTKQPSKSCHKPKAIVHTEKVNMMSLTVLGLRMLFAKTVAVNFLLT |
| | | AKLFFL |

### Example 2. Materials and Methods

### Cells and Cell Lines

PBMCs were isolated from buffy coats obtained from Sanquin Blood Bank (Amsterdam, The Netherlands). Primary AML blasts were received after obtaining informed consent from the LML biobank UMC Utrecht and a kind gift from Matthias Theobald (Mainz, Germany) and were collected according to GCP and Helsinki regulations.

The cell lines Daudi, MM1s, cells expressing NY-ESO-1 were cultured in RPMI (Gibco) supplemented with 10% FCS and 1% pen/strep (Gibco). HEK293FT cell line was cultured in DMEM (Gibco) supplemented with 10% FCS and 1% pen/strep. Human primary T cell clones were cultured in RPMI supplemented with 10% pooled human serum and 1% pen/strep. Human bulk primary T cells used for retroviral transductions were cultured in RPMI, supplemented with 2.5% pooled human serum and 1% pen/strep. All human primary T cells were cultured following the 2-week rapid expansion protocol (REP, including irradiated feeder cells (Daudi, LCL-TM and allogeneic PBMC)+PH A+IL-15+IL-2.

### Mutagenesis, Cloning and Sequencing

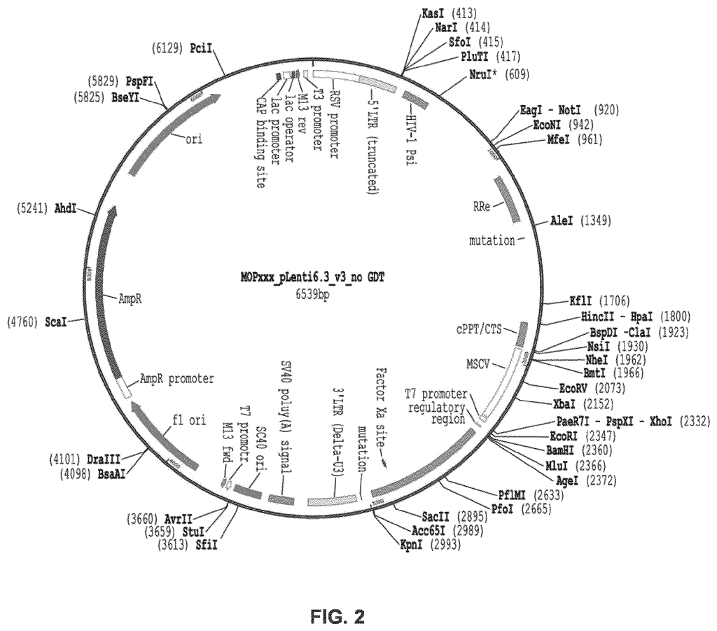

Lentiviral vectors were generated to allow for genomic integration of DNA sequences provided in Example 1, codon optimized DNA encoding for the CAR, full length γ- and δ-chains was generated and subcloned by gBlock gene assembly (−DT-Integrated DNA Technologies, Leuven, Belgium) into pLenti 6.3 lentiviral vector (SEQ ID: 148). Clonal strategy used as HIFI DNA Assembly cloning (NEB, Ipswich, MA, USA). Each active moiety was separated by a T2A or P2A self-cleaving peptide. Expression was driven by a MSCV promoter. Viral genome packaging and transgene expression enhancement were achieved by LTR/Y and WPRE regulatory elements, respectively. Lentiviral particles were produced using the LV-Max system from ThermoFisher Scientific (Waltham, MA, USA). LV-MAX producer cells (A35827) were transfected with pLenti 6.3 γδTCR transfer construct and packaging mix (pLP1, pLP2, pLP-VSVG). Lentiviral titers were assessed in αβTCR-deficient Jurkat-76 cells by flow cytometry analysis, measuring the percentage of CD3/γδTCR among live cells and cloned into pLenti6.3 (FIG. 2).

### Transduction of T-Cells

The constructs as described in Example 1 were transduced into T-cells using the outlined protocol. CAR T cells or TEGs were manufactured starting from αβT-cells enriched by MACS separation from healthy donor leukapheresis material, according to manufacturer instructions. Purified αβT-cells were cultured in TEXMACS medium supplemented with 2.5% human serum (Sanquin), rhIL-7 (20-2000 IU/mL) and rh IL15 (20-200 IU/mL) (both from Miltenyi Biotech, Bergisch Gladbach, Germany), and 1% Penicillin/Streptomycin, and activated using TransAct (Miltenyi Biotec) per manufacturer's recommendations. Cells were transduced with γδTCR LV particles (MOI 5) and then expanded for 12 days in TEXMACS medium, 2.5% human serum, rhIL-7 (20-2000 IU/mL) and rh IL15 (20-200 IU/mL), 1% Penicillin/Streptomycin. At the end of the production, transduction efficiency, T-cell purity (>90% in all cases), and relative expression of T-cell markers CD4 and CD8 were measured by flow cytometry. Cells were then cryopreserved in 1 volume of NaCl 0.9%/5% human serum albumin and 1 volume of Cryostor CS10 (Sigma-Aldrich, Darmstadt, Germany).

### Expression Studies

### Flow Cytometry

Expression of CAR and control constructs were analyzed using flow cytometry. Table 2 provides a list of antibodies used in this study.

Briefly, cells were washed with FACS buffer (PBS with 2% fetal bovine serum) and stained with selected fluorescently-conjugated antibodies in FACS buffer for 30 min at 4° C. After staining, cells were washed twice with FACS buffer, and fixed by 4% paraformaldehyde for 10 min at 4 degrees Celsius. Following fixation, cells were washed once more with FACS buffer, before resuspension in 100-200 μl of FACS buffer, followed by flow cytometric analysis. Mean Fluorescence Intensity (MFI) was calculated.

TABLE 2
List of antibodies used in the study

| | Fluoro- | | | | |
|---|---|---|---|---|---|
| Antigen | chrome | Clone | Organism | Product # | Vendor |
| CD4 | AF700 | RPA-T4 | human | 56-0049-42 | eBioscience/ |
| | | | | | Thermo |
| CD8a | BV510 | RPA-T8 | human | 301048 | Biolegend |
| CD3 | BV421 | UCHT-1 | human | 300434 | Biolegend |
| αβTCR | APC | IP26 | human | 17-9986-42 | eBioscience/ |
| | | | | | Thermo |
| γδTCR | FITC | IMMU510 | human | B49175 | Beckman |
| | | | | | Coulter |
| γδTCR | APC | REA591 | human | 130-113-508 | Miltenyi |
| γδTCR | PE | IMMU510 | human | B49176 | Beckman |
| | | | | | Coulter |
| Fab2 | AF647 | polyclonal | human | 115-605-072 | Jackson |
| | | | | | Immuno- |
| | | | | | research |
| | | | | | Europe |
| | | | | | Ltd\Sanbio |

Vector Copy Number Using qPCR

To determine if expression was linked to higher vector copy number (VCN) a qPCR was performed. For this assay a vector-specific set of primers (SEQ ID NO: 117-119) was designed based on the sequence of the WPRE gene in pLenti 6.3 backbone. An Alb-K primer-set (SEQ ID NO: 120-122; based on the Alb-K gene, see Table 3) was used as positive control and as internal standard for the number of T cells. Samples were washed with PBS and DNA was isolated according to manufacturer protocol (DNAeasy blood & tissue kit, Qiagen, Thermofisher). The VCN was calculated as the ratio of WPRE and Alb-K copy numbers. The qPCR consists of two steps. During step 1, the enzyme polymerase is activated at 95° C. In the actual qPCR step, cDNA is denatured at 95° C. to produce single stranded DNA. Hereafter, Delta and Alb-K products (amplicons) are quantitatively synthesized from cDNA at 62° C. annealing temperatl (Tm) for 30 seconds per cycle using the TaqMan Fast Advanced Master Mix. The PCR step holds 42 cycles referred to as cycle threshold (Ct), being a relative measure of the concentration of Delta and Alb-K in the qPCR reaction. Standard curves in the range 1 to 1E7 copies are prepared by serial dilution of pLenti 6.3 containing one copy each of WPRE and Alb-K gene gDNA in water. qPCR is performed according to a standard two step qPCR program using the TaqMan Fast Advanced Master Mix and annealing set at 62° C. for 30 seconds. VCN1 internal reference sample was generated by lentiviral transduction of Jurkat T cells with 1 copy of the pLenti 6.3 vector.

TABLE 3
List of primers and probes

| SEQ | | |
|---|---|---|
| ID | Primer/ | |
| NOS. | probe name | Sequence |
| 117 | WPRE Fw | TCC TGG TTG CTG TCT CTT TAT G |
| | primer | |
| 118 | WPRE Rv | TGA CAG GTG GTG GCA ATG |
| | primer | |
| 119 | WPRE Probe | TGC ACT GTG TTT GCT GAC GCA AC- |
| | | FAM |
| 120 | Alb-K Fw | GCT GTC ATC TCT TGT GGG CTG T |
| | primer | |
| 121 | Alb-K Rv | ACT CAT GGG AGC TGC TGG TTC |
| | primer | |
| 122 | Alb-K probe | CCT GTC ATG CCC ACA CAA ATC TCT |
| | | CC-VIC |

### Functional T-Cell Assays

### Co-Culture Assays

Fresh (day 12 of production) or cryopreserved T-cells or TEGs were used as effector cells. The number of effector cells added to all co-cultures were corrected to match the cells with the lowest transduction efficiency in the experiment. Untransduced cells which went through the same production process, but without adding lentiviral vectors, were added so that all co-cultures within one experiment contained the same number of of total T cells.

Co-cultures were setup at an effector to target ratio (E:T) of 1:1 or 0.3:1 or otherwise if mentioned, and with or without pamidronate treatment (10 μm) on the day of effector cell addition. Untransduced, effector only, target only, or full lysis controls were included.

After 3 or 7 days, supernatant was collected from the co-cultures, nonadherent cells were resuspended gently, and cell suspension was transferred to round bottom 96 well plate. When cytotoxicity was determined by luminescence, 100 μl of assay medium together with D-luciferin was added to the residual co-culture plate containing any remaining target cells, and incubated for 12 minutes before measuring luminescence by Glomax (Promega). For Xcelligence based cytotoxicity, data was captured in real-time along the co-culture incubation, and after transferring the cell suspension plates were discarded.

Cell suspension was medium exchanged to 60 μl per well, 50 μl of this was transferred δ a new plate of pre-seeded targets for a subsequent round of cytotoxicity evaluation, and the process was repeated as indicated for individual figures.

### Example 3. Results: Comparative Expression Per Cell (MFI-Mean Fluorescence Intensity) of CARs

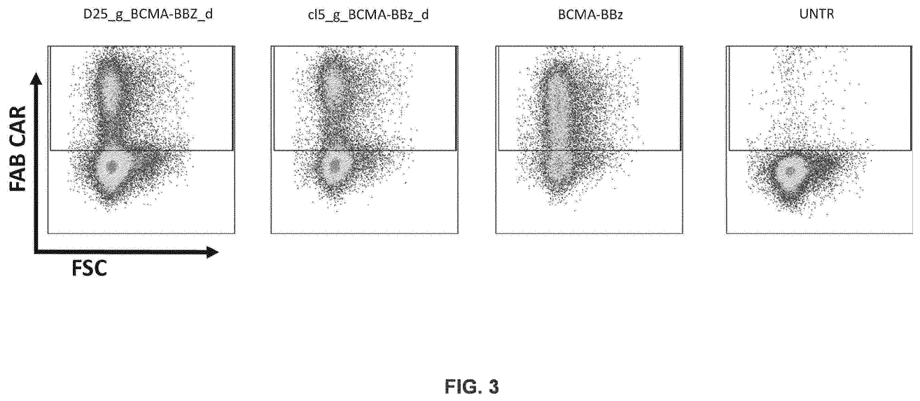

The comparative expression of BCMA-BBz CAR per cell was calculated using the mean fluorescence intensity using the methods provided in Example 2. T-cells expressing BCMA-BBz CAR alone or in a multicistronic construct with a γTCR and δTCR were analyzed for levels of expression using Goat Anti-Mouse I′G, F(ab′)₂ (Sanbio). As shown in Table 4 and FIG. 3, donor cells with BCMA-BBz CAR expressed from a multicistronic construct with γδ-TCR showed higher levels of BCMA-BBz CAR compared to cells expressing BCMA-BBz CAR alone.

TABLE 4
Comparative expression of BCMA-
BBz CAR (VCN—vector copy number)

| | | | | | VCN1 |
|---|---|---|---|---|---|
| | | | | | normalized |
| | MFI | | | | expression/ |
| Cells | CAR+ | CAR+% | VCN | VCN1 | VCN (%) |
| D25_g_BCMA- | 15384 | 30 | 0.63 | 1.10 | 52 |
| BBz_d | | | | | |
| (SEQ ID NO: | | | | | |
| 174) | | | | | |
| cl5_g_BCMA- | 19301 | 42.6 | 1.33 | 1.10 | 35 |
| BBz_d | | | | | |
| (SEQ ID NO: | | | | | |
| 112) | | | | | |
| BCMA-BBz-CAR | 8834 | 68.1 | 1.59 | 0.93 | 40 |
| (SEQ ID NO: | | | | | |
| 114) | | | | | |
| UNTR | 3176 | 1.91 | | | |

The above data indicates that both D25_g_BCMA-BBz_d (SEQ ID NO: 174) and cl5_g_BCMA-BBz_d (SEQ ID NO: 112) exhibit about 2-fold higher levels of CAR expression in comparison to CAR expressed alone.

### Example 4. Results: Cytotoxicity Assays

To determine cytotoxicity co-cultures were setup at an effector to target ratio (E:T) of 1:1 with or without pamidronate treatment (10 μm) on the day of effector cell addition when indicated by +PAM. Untransduced (UNTR), effector only, target only, or full lysis controls were included. Daudi cells were used as target.

After 7 days, supernatant was collected from the co-cultures, nonadherent cells were resuspended gently, and cell suspension was transferred to round bottom 96 well plate. When cytotoxicity was determined by luminescence, 100 μl of assay medium (IMDM medium, 5% human serum, and 1% Penicillin/Streptomycin) together with 10 μl Pierce D-luciferin solution in PBS at concentration of 3 mg/ml (Thermo Fisher) was added to the residual co-culture plate containing any remaining target cells and incubated for 12 minutes before measuring luminescence by Glomax (Promega).

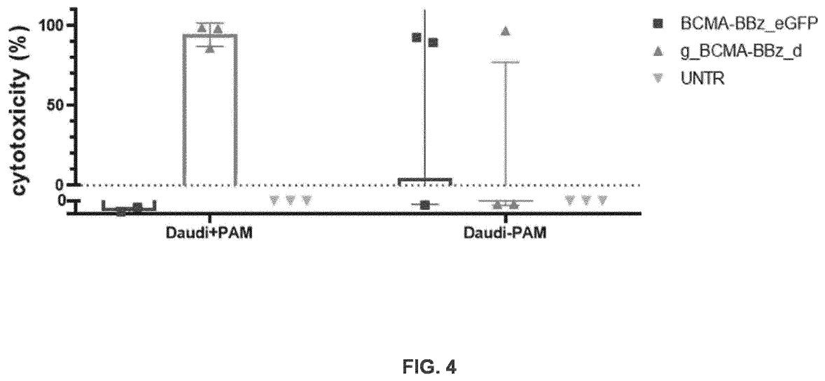

FIG. 4 shows the cytotoxicity data. TEGs expressing g_an-i BCMA-41BBz CAR_d showed >10-fold greater cytotoxicity compared to control cells (UNTR) or CAR-T cells expressing BCMA BBz CAR and eGFP. The above data indicates that TEGs have both a CAR and a functional γδ TCR as additional targeting moiety above BCMA CAR alone.

### Example 5. Results: Cytotoxicity Assay: Effect of Co-Expressing a CAR and γδTCR on Cytotoxicity of Cells Expressing γ and δ BCMA-BBz CAR

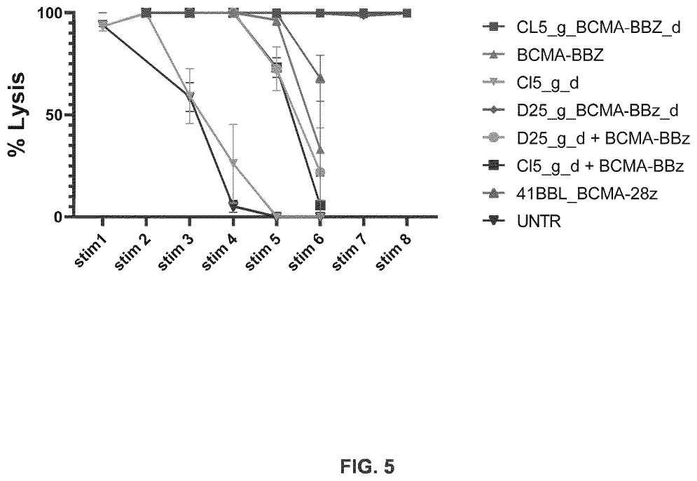

This example evaluates the effect of having γ and δ TCR co-expressed with BCMA-BBz-CAR or separately in two vectors. CAR T cells and TEGs were generated as provided in the methods. The cells were transduced with a multicistronic vector containing a CL5 or D25 γδTCR and BCMA-BBz CAR or 41BBL and BCMA-CD28z CAR; a vector containing CL5 γδTCR or BCMA-BBz CAR alone or were transduced by two vectors, one containing CL5 or D25 γδTCR and one BCMA-BBz CAR. The generated CAR T cells and TEGs, together with untransduced T cells (control,) were co-incubated with BCMA target tumor cells MM1S which are not recognized by the gamma-delta TCR. Every three days T cells were transferred to fresh target cells and residual target cell viability was measurement by luciferase assay. Serial stimulation of TEGs was continued for 8 stimulations. Data for the assay are provided in FIG. 5. Both multicistronic constructs CL5_g_BCMA-BBz_d and D25_g_BCMA-BBz_d exhibited 100% cytolysis in these assays for up to 8 stimulations for which the data was collected. This was multiple stimulation rounds more than any of the other conditions. This data demonstrates that expressing a CAR together with a γδTCR in a multicistronic setting has superior functionality above CAR alone, even when costimulation is provided.

### Example 6. Results: Effect of Co-Expressing a CAR and γδTCR on Expression of BCMA-CAR

This example further evaluates the expression of BCMA-CAR in T-cells cells expressing the BCMA-BBz CAR with and without co-expression of different γδ-TCR subunits and using different cis-regulatory domains (e.g., P2A vs. T2A). T cells were transduced with lentivirus constructs expressing BCMA-CAR (SEQ ID NO: 159 encoding the polypeptide of SEQ ID NO: 160), BCMA-CAR-P2A (SEQ ID NO: 153 encoding the polypeptide of SEQ ID NO: 154), CI5-BCMA-CAR-P2A (SEQ ID NO: 111 encoding the polypeptide of SEQ ID NO: 112), BCMA-CAR-T2A (SEQ ID NO: 113, encoding the polypeptide of SEQ ID NO: 114), or CI5-BCMA-CAR-T2A (SEQ ID NO: 151 encoding the polypeptide of SEQ ID NO: 152). Each of these multi-cistronic constructs were prepared as described above in the Methods.

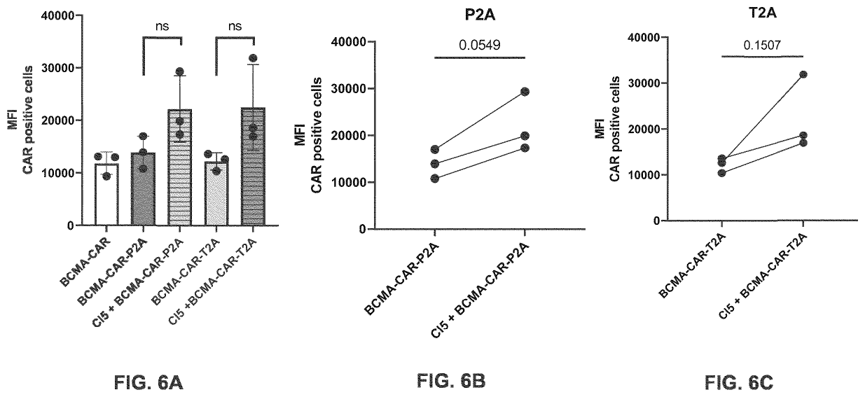

Following transduction, the mean fluorescent intensity (MFI) of BCMA-CAR expression was analyzed and compared for each of the transfected constructs. FIG. 6A-6C summarize data from three donors in a single experiment and show that there was no significant difference in BCMA-CAR expression between cells expressing BCMA CAR alone or in construct with the CI5 γδTCR receptor and the identity of the 2A self-cleaving peptide used (e.g., P2A or T2A) also did not affect overall expression.

### Example 7. Results: Cytotoxicity of T-Cells Co-Expressing a CAR and γδTCR on Tumor Cells

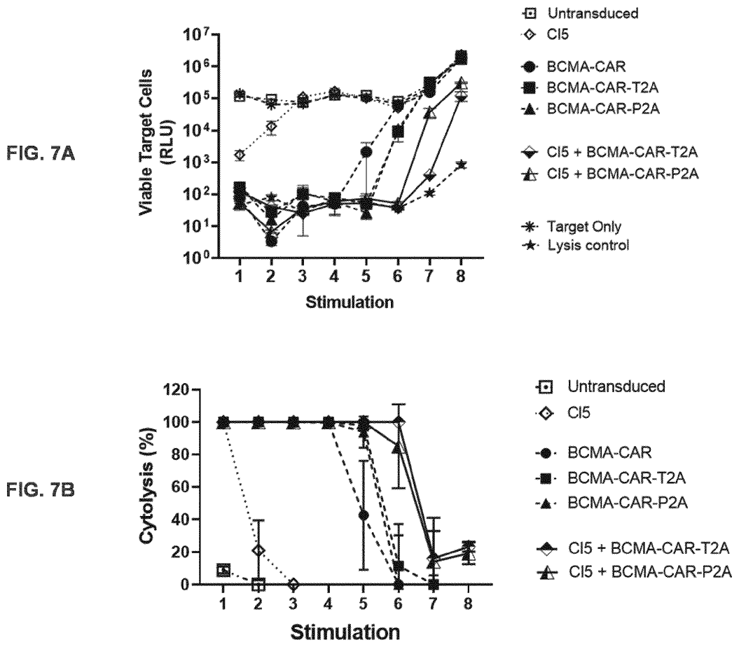

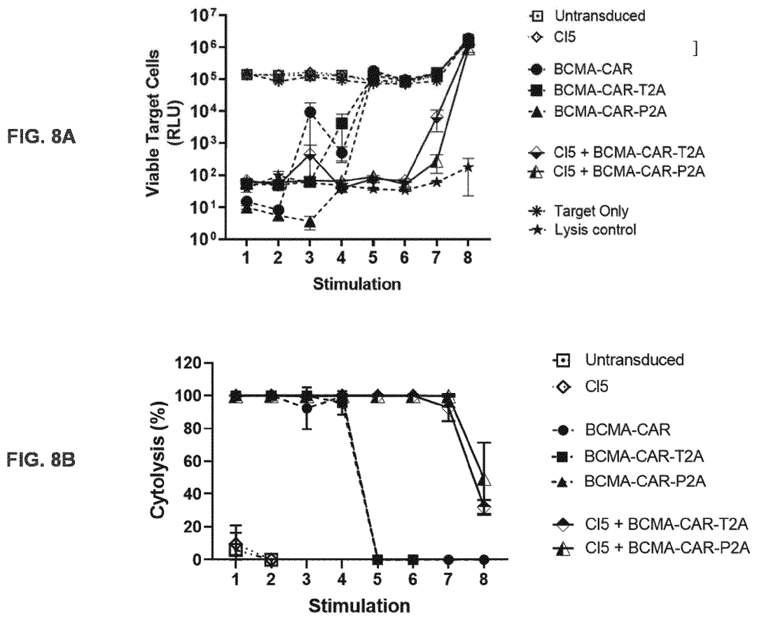

The cytotoxicity of the transduced cells from Example 6 was then tested on RPMI-8226 LucTOM target cells (that express BCMA). Specifically, T cells expressing the constructs listed above (e.g., BCMA-CAR alone or with the γδ-TCR subunits, and with and without P2A and T2A cis-regulatory domains) were co-cultured with RPMI-8226 LucTOM tumor cells which are not recognized by the gamma-delta TCR and effector cells. An effector to target cell ratio of 1:1 was used. Every 3 to 4 days, the transduced T cells were transferred to fresh target cells. Serial stimulation of T cells was continued for 8 stimulations and luciferase levels were observed as a measure of cell lysis. FIG. 7A-7B and FIG. 8A-8B shows mean relative luciferase light unit value for each stimulation across two separate experiments (FIG. 7A/7B and FIG. 8A/8B) as a measure of total cells (FIG. 7A, 8A) and normalized as a measure of percentage of cytolysis (FIG. 7B, 8B). Both multicistronic constructs CI5+BCMA-CAR-T2A and CI5+BCMA-CAR-P2A exhibited 100% cytolysis in these assays for up to 6 or 7 stimulations for which the data was collected which was at least one stimulation round more than cells expressing BCMA-CAR alone (see FIGS. 7B and 8B). This data demonstrates that expressing a CAR together with a γδTCR in a multicistronic setting has superior functionality above CAR alone, even when costimulation is provided.

### Example 8. Results: Cytotoxicity and IFNg Production of Target Cells Using T-Cells Co-Expressing a CEA-CAR and γδTCR

This example further evaluates the cytotoxicity of a different CAR (e.g., CEA-CAR) in T-cells expressing the CEA-CAR with and without co-expression of different γδ-TCR subunits. T cells were transduced with lentivirus expressing CI5, CEA-CAR-P2A or CI5+CEA-CAR-P2A constructs as provided above (see SEQ ID NO: 155 encoding polypeptide of SEQ ID NO: 156 (CEA-CAR-P2A) and SEQ ID NO: 157 encoding polypeptide of SEQ ID NO: 158 (CI5-CEA-CAR-P2A). Each of these multi-cistronic constructs were prepared as described above in the Methods.

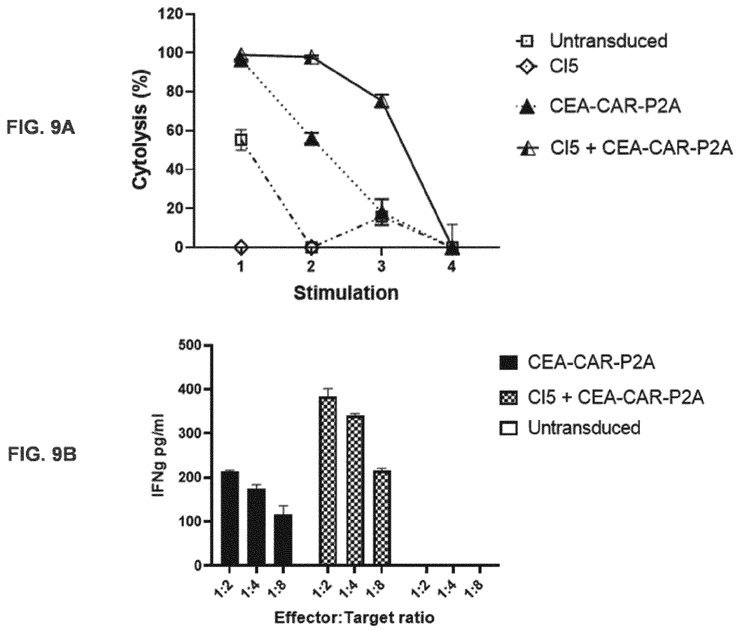

The cytotoxicity of these transduced cells was then tested on HT-29 LucTOM target cells (that express human CEA). Specifically, HT-29 LucTOM tumor cells, which are not recognized by the gamma-delta TCR, were co-cultured with indicated T cells with an effector to target cell ratio of 1:1-1:8. Every 3 to 4 days T cells were transferred to fresh target cells. Serial stimulation of T-cells was continued for 4 stimulations. The mean percentage of lysis of target cells±SD of triplicates, in a single experiment when an E:T of 1:1 was used is shown in FIG. 9A. It was found that co-expression of the CEA-CAR and the γδ-TCR subunits resulted in increased cell lysis over more stimulations than cells expressing CEA-CAR alone.

The amount of IFN release produced by activated T cells after stimulation round 1 when a 1:1-1:8 E/T ratio was used was then determined by ELISA. Specifically, cell culture supernatants from Luciferase-based cytotoxicity assays were harvested at the end of the co-culture to measure IFN-γ secretion using a commercial Human IFN-gamma DuoSet ELISA assay (cat nr. DY285B-05, R&D Systems, Minneapolis, MN, US), according to manufacturer's instructions. This is a standard sandwich ELISA using a plate-bound capture antibody and a detection antibody both specific for IFN-γ. The detection antibody is linked to an enzyme which can convert a substrate into an absorbance signal which is measured with a plate reader. The internal standard curve allows absorbance values to be calculated into the IFN-γ concentration (pg/mL) released into the supernatants. FIG. 9B shows levels of IFN-γ measured for indicated effector/T cell ratios and demonstrates that co-transfection of the CEA-CAR with the γδTCR results in higher levels of IFN-γ across all effector: target cells ratios tested.

1. A polynucleotide comprising nucleic acids encoding each of the monomers of a heterodimeric γδT-cell receptor or functional fragments thereof, and at least one nucleic acid encoding a chimeric antigen receptor (CAR), wherein said nucleic acids are operably linked to the same promoter sequence.

2. A polynucleotide according to claim 1, wherein said at least one nucleic acid encoding said chimeric antigen receptor (CAR) is inserted between the nucleic acids encoding each of said monomers.

3. A polynucleotide according to claim 1, wherein said at least one nucleic acid encoding said chimeric antigen receptor (CAR) is inserted downstream or upstream of the nucleic acids encoding each of said monomers.

4. A polynucleotide according to any one of claims 1-3, wherein said promoter sequence is selected from the group consisting of EF1α, MSCV, EF1 alpha-HTLV-1 hybrid promoter, Moloney murine leukemia virus, Gibbon Ape Leukemia virus, murine mammary tumor virus, Rous sarcoma virus, MHC class II, clotting Factor IX, insulin promoter, PDX1 promoter, CD11, CD4, CD2, gp47 promoter, PGK, Beta-globin, UbC, and MND, preferably from MSCV, MMLV, EF1α, and MND.

5. A polynucleotide according to any one of claims 1-4, wherein said polynucleotide further comprises at least one nucleic acid encoding at least one cis-acting regulatory element which facilitates co-expression of the CAR and the γδT-cell receptor.

6. A polynucleotide according to claim 5, wherein said at least one cis-acting regulatory element is selected from a 2A self-cleaving peptide, a 2A-like cis-acting hydrolase element and an IRES sequence.

7. A polynucleotide according to claim 6, wherein said 2A self-cleaving peptide is selected from a T2A, a P2A, an E2A, and an F2A peptide.

8. A polynucleotide according to claim 7, comprising a first cis-acting regulatory element and a second cis-acting regulatory element.

9. A polynucleotide according to claim 8, wherein said first cis-acting regulatory element is a T2A peptide and said second cis-acting regulatory element is a P2A peptide.

10. A polynucleotide according to claim 8, wherein said first and second cis-acting regulatory elements are each independently selected from the group consisting of Picornavirus IRES, Apthovirus IRES, Hepatitis A IRES, Pestivirus IRES, Hepesvirus IRES, and combinations thereof.

11. A polynucleotide according to any preceding claim, wherein said polynucleotide is multicistronic.

12. A polynucleotide according to any preceding claim, wherein said polynucleotide is tricistronic or tetracistronic.

13. A polynucleotide according to any preceding claim, wherein said CAR comprises: (i) a CAR antigen binding domain; (ii) a transmembrane domain; and (iii) an intracellular cytoplasmic binding domain; and optionally further comprises at least one of (iv) a stalk (spacer) domain; and (v) a co-stimulatory domain.

14. A polynucleotide according to claim 13, wherein said CAR antigen binding domain specifically binds to a tumor antigen.

15. A polynucleotide according to claim 13 or 14, wherein said CAR antigen binding domain comprises any one of an ScFV, Fab, a nanobody or any natural binding partner or fragment thereof of the tumor antigen.

16. A polynucleotide according to claim 14 or 15, wherein the tumor antigen is selected from the group consisting of: BCMA, CD19, PSA, HER-2/neu, MUC1, Carcinoembryonic antigen (CEA), CA-125, Epithelial tumor antigen (ETA), Tyrosinase, Melanoma-associated antigen (MAGE).

17. The polynucleotide according to any one of claims 13 to 16, wherein the tumor antigen comprises an amino acid sequence having at least 60%, 70%, 75%, 80%, 85%, 90%, 95%, 96%, 97%, 98%, 99%, or 100% sequence identity or similarity to any one of SEQ ID NOs: 1-2 and 162.

18. The polynucleotide according to any one of claims 13 to 17, wherein the CAR antigen binding domain comprises an amino acid sequence having at least 60%, 70%, 75%, 80%, 85%, 90%, 95%, 96%, 97%, 98%, 99%, or 100% sequence identity or similarity to any one of SEQ ID NOs: 163-165.

19. The polynucleotide according to any one of claims 13 to 18 wherein said transmembrane domain comprises a CD8 transmembrane domain, a CD137 (4-1BB transmembrane domain) or a CD28 transmembrane domain, and optionally comprises an amino acid sequence having at least 60%, 70%, 75%, 80%, 85%, 90%, 95%, 96%, 97%, 98%, 99%, or 100% sequence identity or similarity to any one of SEQ ID NO: 5-7.

20. A polynucleotide according to any one of claims 13-19, wherein said costimulatory domain comprises the intracellular domain of a costimulatory molecule selected from the group consisting of CD27, CD28, 4-1BB, CD30, and any combination thereof.

21. The polynucleotide of claim 20, wherein the intracellular domain of a costimulatory molecule comprises an amino acid sequence having at least 60%, 70%, 75%, 80%, 85%, 90%, 95%, 96%, 97%, 98%, 99%, or 100% sequence identity or similarity to any one of SEQ ID NOS: 10 or 11.

22. A polynucleotide according to any one of claims 13-21, wherein said intracellular cytoplasmic binding domain comprises a domain selected from a group consisting of CD3e, CD3z, and any domain comprising an ITAM or hemiTAM.

23. The polynucleotide of claim 22, wherein the intracellular cytoplasmic binding domain comprises an amino acid sequence having at least 60%, 70%, 75%, 80%, 85%, 90%, 95%, 96%, 97%, 98%, 99%, or 100% sequence identity or similarity to SEQ ID NO: 8 or 9.

24. A polynucleotide according to any one of claims 13-23, wherein said stalk domain comprises a CD8 or CD28 stalk.

25. The polynucleotide of claim 24, wherein the stalk domain comprises a sequence which has at least 60%, 70%, 75%, 80%, 85%, 90%, 95%, 96%, 97%, 98%, 99%, or 100% sequence identity or similarity to SEQ ID NO: 3 or 4.

26. The polynucleotide of any one of claims 13 to 16, wherein the CAR comprises an amino acid sequence having at least 60%, 70%, 75%, 80%, 85%, 90%, 95%, 96%, 97%, 98%, 99%, or 100% sequence identity or similarity to any one of SEQ ID NOs: 29, 30 and 31.

27. The polynucleotide of any one of claims 1 to 26, wherein the heterodimeric γδT-cell receptor comprises: a) a γ-chain selected from the group consisting of γ2, γ3, γ4, γ5, γ8, γ9, and γ11, and derivatives, variations or fragments thereof; (b) a δ-chain selected from the group consisting of δ1, δ2, δ3, and δ5, and derivatives, variations or fragments thereof, or (c) any combination of (a) and (b).

28. The polynucleotide according to claim 27, wherein the heterodimeric γδT-cell receptor comprises a γ9δ2 or a γ4δ5 receptor.

29. The polynucleotide of claim 27 or 28, wherein the heterodimeric γδT-cell receptor comprises a γ-chain having a complementary determining region 3 (CDR3) having at least 60%, 70%, 75%, 80%, 85%, 90%, 95%, 96%, 97%, 98%, 99%, or 100% sequence identity or similarity with an amino acid sequence of SEQ ID NO: 37 and/or a δ-chain having a complementary determining region 3 (CDR3) having at least 60%, 70%, 75%, 80%, 85%, 90%, 95%, 96%, 97%, 98%, 99%, or 100% sequence identity or similarity with an amino acid sequence of SEQ ID NO: 34.

30. The polynucleotide of claim 29, wherein:
(a) the γ-chain of the heterodimeric γδT-cell receptor further comprises a complementary determining region 1 (CDR1) having at least 60%, 70%, 80%, 90%, 95%, or 100% identity or similarity with an amino acid sequence of SEQ ID NO: 166, a complementary determining region 2 (CDR2) having at least 60%, 70%, 75%, 80%, 85%, 90%, 95%, 96%, 97%, 98%, 99%, or 100% sequence identity or similarity with an amino acid sequence of SEQ ID NO: 167, or a combination thereof; and/or
(b) the δ-chain further comprises a complementary determining region 1 (CDR1) having at least 60%, 70%, 80%, 90%, 95%, or 100% identity or similarity with an amino acid sequence of SEQ ID NO: 168, a complementary determining region 2 (CDR2) having at least 60%, 70%, 75%, 80%, 85%, 90%, 95%, 96%, 97%, 98%, 99%, or 100% sequence identity or similarity with an amino acid sequence of EKD or a combination thereof.

31. The polynucleotide of any one of claims 28-30, wherein the γ-chain of the heterodimeric γδT-cell receptor comprises an amino acid sequence having at least 60%, 70%, 80%, 90%, 95%, or 100% identity or similarity with SEQ ID NO: 41; and/or the δ-chain of the heterodimeric γδT-cell receptor comprise an amino acid sequence having at least 60%, 70%, 75%, 80%, 85%, 90%, 95%, 96%, 97%, 98%, 99%, or 100% sequence identity or similarity with SEQ ID NO: 40.

32. The polynucleotide of claim 27 or 28, wherein the heterodimeric γδT-cell receptor comprises a y-chain having a complementary determining region 3 (CDR3) having at least 60%, 70%, 80%, 90%, 95%, or 100% identity or similarity with an amino acid sequence of SEQ ID NO: 51; and/or a δ-chain having a complementary determining region 3 (CDR3) having at least 60%, 70%, 75%, 80%, 85%, 90%, 95%, 96%, 97%, 98%, 99%, or 100% sequence identity or similarity with an amino acid sequence of SEQ ID NO: 52.

33. The polynucleotide of claim 32, wherein:
(a) the γ-chain of the heterodimeric γδT-cell receptor further comprises a complementary determining region 1 (CDR1) having at least 60%, 70%, 80%, 90%, 95%, or 100% identity or similarity with an amino acid sequence of SEQ ID NO: 169, a complementary determining region 2 (CDR2) having at least 60%, 70%, 75%, 80%, 85%, 90%, 95%, 96%, 97%, 98%, 99%, or 100% sequence identity or similarity with an amino acid sequence of SEQ ID NO: 170, or a combination thereof; and/or
(b) the δ-chain further comprises a complementary determining region 1 (CDR1) having at least 60%, 70%, 80%, 90%, 95%, or 100% identity or similarity with an amino acid sequence of SEQ ID NO: 171, a complementary determining region 2 (CDR2) having at least 60%, 70%, 75%, 80%, 85%, 90%, 95%, 96%, 97%, 98%, 99%, or 100% sequence identity or similarity with an amino acid sequence of SEQ ID NO: 172, or a combination thereof.

34. The polynucleotide of any one of claims 28-30, wherein the γ-chain of the heterodimeric γδT-cell receptor comprises an amino acid sequence having at 60%, 70%, 75%, 80%, 85%, 90%, 95%, 96%, 97%, 98%, 99%, or 100% sequence identity or similarity with SEQ ID NO: 62; and/or the δ-chain of the heterodimeric γδT-cell receptor comprise an amino acid sequence having at least 60%, 70%, 75%, 80%, 85%, 90%, 95%, 96%, 97%, 98%, 99%, or 100% sequence identity or similarity with SEQ ID NO: 61.

35. A polynucleotide according to any preceding claim, wherein said γδT-cell receptor binds to a first antigen and said CAR binds to a second antigen.

36. The polynucleotide according to claim 35, wherein the γδT-cell receptor binds to cluster of differentiation 277 (CD277), an endothelial protein c receptor (EPCR), an annexin A2 protein or any combination thereof; and the CAR binds to B cell maturation antigen (BCMA), cluster of differentiation 19 (CD19), carcinoembryonic antigen (CEA) or any combination thereof.

37. A polynucleotide according to any preceding claim, wherein one or both of said monomers of the heterodimeric γδT-cell receptor have a deletion of all or a portion of the variable domain.

38. A polynucleotide according to any preceding claim, wherein the polynucleotide encodes a polypeptide having at least 60%, 70%, 75%, 80%, 85%, 90%, 95%, 96%, 97%, 98%, 99%, or 100% sequence identity or similarity with any one of SEQ ID NOs: 112, 152, 158, and 174.

39. A vector comprising the polynucleotide according to any preceding claim.

40. A vector according to claim 39, wherein said vector comprises a plasmid, a retroviral vector, lentiviral vector, an adenoviral vector, or an adenovirus associated viral (AAV) vector.

41. A vector according to claim 39 or 40, comprising a nucleic acid sequence having at least 60%, 70%, 75%, 80%, 85%, 90%, 95%, 96%, 97%, 98%, 99%, or 100% sequence identity or similarity with any one of SEQ ID NO:111, 151, 157 and 173.

42. An immune cell comprising a polynucleotide according to any one of claims 1-38, or a vector according to any one of claims 39-41.

43. An immune cell according to claim 42, which is a T cell.

44. An immune cell according to claim 43 wherein the T cell is an αβ T-cell.

45. An immune cell according to any one of claims 42-44, wherein said cell co-expresses said γδT-cell receptor and said CAR.

46. An immune cell according to any one of claims 42-44, wherein the level of CAR expression in said cell is increased relative to the level of CAR expression in a cell lacking co-expression of said γδT-cell receptor and said CAR.

47. A population of cells comprising a plurality of immune cells according to any one of claims 42 to 46.

48. An immune cell according to any one of claims 42-46, or population of cells according to claim 47, wherein with respect to a tumor cell having an antigen that binds to said CAR of said cells, said cells exhibit any one or more of increased cytotoxicity, antitumor activity, and/or tumor cell killing, and/or proliferation, cellular survival, or persistence, as compared to cells that do not co-express said γδT-cell receptor and said CAR.

49. A method of increasing expression and/or function of a CAR in an immune cell, said method comprising introducing to the immune cell the polynucleotide according to any one of claims 1-38, or the vector according to any one of claims 39-41.

50. A method according to claim 49, wherein the immune cell is a T cell.

51. A method according to claim 50, wherein the T cell is an αβ T cell.

52. A method according to any one of claims 49-51, wherein increasing function of the CAR in the immune cell comprises increasing any one or more of cytotoxicity, antitumor activity, and/or tumor cell killing with respect to a cancer cell having an antigen that binds to the CAR of the cells, and/or increasing proliferation, cellular survival, or persistence of the immune cell, as compared to an immune cell that does not co-express said γδT-cell receptor with the CAR.

53. A method according to any one of claims 49-52, further comprising maintaining the immune cell for a time and under conditions sufficient for the immune cell to co-express the γδT-cell receptor and the CAR.

54. The method of any one of claims 49-53, wherein increasing expression of the CAR in the immune cell comprises increasing the level of CAR expression in the immune cell relative to the level of CAR expression in an immune cell lacking co-expression of the γδT-cell receptor.

55. A method for treating a cancer in a subject in need thereof, said method comprising: administering to the subject an effective amount of an immune cell according to any one of claims 42-46 and 48, or a population of immune cells according to any one of claims 47-48, thereby inducing anti-cancer immunity in the subject.

56. A method for stimulating an immune cell-mediated immune response to a cell population in a subject in need thereof, said method comprising: administering to the subject an effective amount of immune cells according to any one of claims 42-46 and 48, or a population of immune cells according to any one of claims 47-48, thereby stimulating in the subject am immune cell-mediated immune response to the cell population.

57. A method according to claim 55 or 56, wherein the subject is a mammal.

58. A method according to claim 55 or 56, wherein the subject is a human.

59. One or more immune cells according to any one of claims 42-46 and 48, or a population of immune cells according to any one of claims 47-48, for use in the treatment of a cancer.

60. One or more immune cells according to any one of claims 42-46 and 48, or a population of immune cells according to any one of claims 47-48, for use in stimulating an immune cell-mediated immune response to a cell population or tissue.

61. A polynucleotide comprising A or B, wherein:
a. (A) is a nucleic acid represented by (i)-(ii)-(iii), wherein:
(i) is a nucleic acid encoding a γ chain of a γδT-cell receptor or a functional fragment thereof,
(ii) is at least one nucleic acid encoding a CAR or a functional fragment thereof, and;
(iii) is a nucleic acid encoding a δ chain of a γδT-cell receptor or a functional fragment thereof,
wherein (ii) is positioned between (i) and (iii), is positioned downstream of both (i) and (iii), or is positioned upstream of both (i) and (iii),
b. (B) is a nucleic acid represented by (iv)-(v)-(vi), wherein:
(iv) is a nucleic acid encoding a δ chain of a γδT-cell receptor or a functional fragment thereof,
(v) is at least one nucleic acid encoding a CAR or a functional fragment thereof, and;
(vi) is a nucleic acid encoding a γ chain of a γδT-cell receptor or a functional fragment thereof,
wherein (v) is positioned between (iv) and (vi), is positioned downstream of both (iv) and (vi), or is positioned upstream of both (iv) and (vi).

62. The polynucleotide of claim 61, wherein:
(i) and (vi) are nucleic acids comprising a nucleotide sequence encoding a polypeptide having at least 60%, 70%, 75%, 80%, 85%, 90%, 95%, 96%, 97%, 98%, 99%, or 100% sequence identity or similarity with an amino acid sequence selected from SEQ ID NOs: 35, 36, 37, 39, 41, 43, 44, 45, 46, 51, 54, 56, 58, 60, 62, 63, 65, 67, 69, 71, 73, 75, 77, 79, 80, 82, 86, 87, 90, 91, 94, 95, 98, 99, 101, 102, 125, 126, 129, 130, 133, 134, 137, 138, 141, 142, 145, 146, 149, and 150, preferably selected from SEQ ID NOs: 35, 36, 37, 44, 45, 46, 51, 63, 65, 67, 69, 77, 80, 86, 87, 90, 91, 94, 95, 98, 99, 101, 102, 125, 126, 129, 130, 133, 134, 137, 138, 141, 142, 145, 146, 149, and 150 and/or;
(iii) and (iv) are nucleic acids comprising a nucleotide sequence encoding a polypeptide having at least 60%, 70%, 75%, 80%, 85%, 90%, 95%, 96%, 97%, 98%, 99%, or 100% sequence identity or similarity with an amino acid sequence selected from SEQ ID NOs: 32, 33, 34, 38, 40, 42, 47, 48, 49, 50, 52, 53, 55, 57, 59, 61, 64, 66, 68, 70, 72, 74, 76, 78, 81, 83, 84, 85, 88, 89, 92, 93, 96, 97, 100, 123, 124, 127, 128, 131, 132, 135, 136, 139, 140, 143, 144, 147, and 148 preferably selected from SEQ ID NOs: 32, 33, 34, 47, 48, 49, 50, 52, 64, 66, 68, 76, 81, 84, 85, 88, 89, 92, 93, 96, 97, 100, 123, 124, 127, 128, 131, 132, 135, 136, 139, 140, 143, 144, 147, and 148.

63. The polynucleotide of claim 61 or 62, wherein:
(i) and (vi) are nucleic acids comprising a nucleotide sequence encoding a polypeptide having at least 60%, 70%, 80%, 90%, 95%, or 100% identity or similarity with an amino acid sequence of SEQ ID NOs: 37 and, optionally, further comprising a nucleotide sequence encoding a polypeptide having at least 60%, 70%, 75%, 80%, 85%, 90%, 95%, 96%, 97%, 98%, 99%, or 100% sequence identity or similarity to SEQ ID NO: 166 and/or a nucleotide sequence encoding a polypeptide having at least 60%, 70%, 75%, 80%, 85%, 90%, 95%, 96%, 97%, 98%, 99%, or 100% sequence identity or similarity to SEQ ID NO: 167, and/or;
(iii) and (iv) are nucleic acids comprising a nucleotide sequence encoding a polypeptide having at least 60%, 70%, 75%, 80%, 85%, 90%, 95%, 96%, 97%, 98%, 99%, or 100% sequence identity or similarity with an amino acid sequence of SEQ ID NO: 34 and, optionally, further comprising a nucleotide sequence encoding a polypeptide having at least 60%, 70%, 75%, 80%, 85%, 90%, 95%, 96%, 97%, 98%, 99%, or 100% sequence identity or similarity to SEQ ID NO: 168 and/or a nucleotide sequence encoding a polypeptide having at least 60%, 70%, 75%, 80%, 85%, 90%, 95%, 96%, 97%, 98%, 99%, or 100% sequence identity or similarity to an amino acid sequence of EKD.

64. The polynucleotide of claim 61 or 62, wherein: (i) and (vi) are nucleic acids comprising a nucleotide sequence encoding a polypeptide having at least 60%, 70%, 75%, 80%, 85%, 90%, 95%, 96%, 97%, 98%, 99%, or 100% sequence identity or similarity with an amino acid sequence of SEQ ID NOs: 41 and/or iii) and (iv) are nucleic acids comprising a nucleotide sequence encoding a polypeptide having at least 60%, 70%, 75%, 80%, 85%, 90%, 95%, 96%, 97%, 98%, 99%, or 100% sequence identity or similarity with an amino acid sequence of SEQ ID NO: 40.

65. The polynucleotide of claim 61 or 62, wherein:
(i) and (vi) are nucleic acids comprising a nucleotide sequence encoding a polypeptide having at least 60%, 70%, 80%, 90%, 95%, or 100% identity or similarity with an amino acid sequence of SEQ ID NOs: 51 and, optionally, further comprising a nucleotide sequence encoding a polypeptide having at least 60%, 70%, 75%, 80%, 85%, 90%, 95%, 96%, 97%, 98%, 99%, or 100% sequence identity or similarity to SEQ ID NO: 169 and/or a nucleotide sequence encoding a polypeptide having at least 60%, 70%, 75%, 80%, 85%, 90%, 95%, 96%, 97%, 98%, 99%, or 100% sequence identity or similarity to SEQ ID NO: 170, and/or;
(iii) and (iv) are nucleic acids comprising a nucleotide sequence encoding a polypeptide having at least 60%, 70%, 80%, 90%, 95%, or 100% identity or similarity with an amino acid sequence of SEQ ID NO: 52 and, optionally, further comprising a nucleotide sequence encoding a polypeptide having at least 60%, 70%, 75%, 80%, 85%, 90%, 95%, 96%, 97%, 98%, 99%, or 100% sequence identity or similarity to SEQ ID NO: 171 and/or a nucleotide sequence encoding a polypeptide having at least 60%, 70%, 75%, 80%, 85%, 90%, 95%, 96%, 97%, 98%, 99%, or 100% sequence identity or similarity to SEQ ID NO: 172.

66. The polynucleotide of claim 61 or 62, wherein: (i) and (vi) are nucleic acids comprising a nucleotide sequence encoding a polypeptide having at least 60%, 70%, 75%, 80%, 85%, 90%, 95%, 96%, 97%, 98%, 99%, or 100% sequence identity or similarity with an amino acid sequence of SEQ ID NOs: 62 and/or iii) and (iv) are nucleic acids comprising a nucleotide sequence encoding a polypeptide having at least 60%, 70%, 75%, 80%, 85%, 90%, 95%, 96%, 97%, 98%, 99%, or 100% sequence identity or similarity with an amino acid sequence of SEQ ID NO: 61.

67. A polynucleotide of any one of claims 61 to 66, wherein (ii) or (v) are nucleic acids comprising a nucleotide sequence encoding a polypeptide having at least 60%, 70%, 75%, 80%, 85%, 90%, 95%, 96%, 97%, 98%, 99%, or 100% sequence identity or similarity to any one of SEQ ID NOs: 3, 4, 5, 6, 7, 8, 9, 10, 11, 29, 30, 31, 154, 156, 160, 163, 164, and 165.

68. A polynucleotide of claim 67, wherein (ii) or (v) are nucleic acids comprising a nucleotide sequence encoding a polypeptide having at least 60%, 70%, 75%, 80%, 85%, 90%, 95%, 96%, 97%, 98%, 99%, or 100% sequence identity or similarity to any one of SEQ ID NOs: 29, 30 or 31.

69. A polynucleotide of any one of claims 61-68, wherein the polynucleotide encodes a polypeptide having a polypeptide having at least 60%, 70%, 75%, 80%, 85%, 90%, 95%, 96%, 97%, 98%, 99%, or 100% sequence identity or similarity with any one of SEQ ID NOs: 112, 152, 158 and 174.

70. A vector comprising a polynucleotide according to any one of claims 61-69.

71. A vector of claim 70, comprising a nucleic acid sequence having at least 60%, 70%, 75%, 80%, 85%, 90%, 95%, 96%, 97%, 98%, 99%, or 100% sequence identity or similarity with any one of SEQ ID NO: 111, 151, 157 and 173.

72. A vector according to claim 70 or 71, wherein said vector comprises a plasmid, a retroviral vector, lentiviral vector, an adenoviral vector, or an adenovirus associated viral (AAV) vector.

73. An immune cell comprising a polynucleotide according to any one of claims 61-69, or a vector according to any one of claims 70-72.

74. An immune cell according to claim 73, which is a T cell.

75. An immune cell according to claim 74, which is an ap T-cell.

76. A method of increasing expression and/or function of a CAR in an immune cell, said method comprising: introducing to the immune cell the polynucleotide according to any one of claims 61-69, or the vector according to any one of claims 70-72.

77. A method according to claim 76, wherein the immune cell is a T cell.

78. A method according to claim 77, wherein the immune cell is an ap T-cell.

79. One or more immune cells according to any one of claims 73 to 75, for use in the treatment of a cancer.

80. One or more immune cells according to any one of claims 73-75, for use in stimulating an immune cell-mediated immune response to a cell population or tissue.


SEQUENCE LISTING (174 sequences)

SEQ ID NO: 1 - protein, 184 aa, Homo sapiens
MLQMAGQCSQNEYFDSLLHACIPCQLRCSSNTPPLTCQRYCNASVTNSVKGTNAILWTCLGLSLIISLAVFVLMFLLRKINSEPLKDEFKNTGSGLLGMANIDLEKSRTGDEIILPRGLEYTVEECTCEDCIKSKPKVDSDHCFPLPAMEEGATILVTTKTNDYCKSLPAALSATEIEKSISAR

SEQ ID NO: 2 - protein, 556 aa, Homo sapiens [sequence omitted]

SEQ ID NO: 3 - protein, 45 aa, synthetic construct
TTTPAPRPPTPAPTIASQPLSLRPEACRPAAGGAVHTRGLDFACD

SEQ ID NO: 4 - protein, 40 aa, synthetic construct
KIEVMYPPPYLDNEKSNGTIIHVKGKHLCPSPLFPGPSKP

SEQ ID NO: 5 - protein, 24 aa, synthetic construct
IYIWAPLAGTCGVLLLSLVITLYC

SEQ ID NO: 6 - protein, 27 aa, synthetic construct
IISFFLALTSTALLFLLFFLTLRFSVV

SEQ ID NO: 7 - protein, 27 aa, synthetic construct
FWVLVVVGGVLACYSLLVTVAFIIFWV

SEQ ID NO: 8 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 40 aa, synthetic construct
SKRSRLLHSDYMNMTPRRPGPTRKHYQPYAPPRDFAAYRS

SEQ ID NO: 11 - protein, 42 aa, synthetic construct
KRGRKKLLYIFKQPFMRPVQTTQEEDGCSCRFPEEEEGGCEL

SEQ ID NO: 12 - protein, 4 aa, synthetic construct
TSGS

SEQ ID NO: 13 - protein, 5 aa, synthetic construct
GGGGS

SEQ ID NO: 14 - protein, 4 aa, synthetic construct
GGGS

SEQ ID NO: 15 - skipped

SEQ ID NO: 16 - protein, 18 aa, synthetic construct
KESGSVSSEQLAQFRSLD

SEQ ID NO: 17 - protein, 14 aa, synthetic construct
EGKSSGSGSESKST

SEQ ID NO: 18 - protein, 12 aa, synthetic construct
GSAGSAAGSGEF

SEQ ID NO: 19 - protein, 5 aa, synthetic construct
EAAAK

SEQ ID NO: 20 - protein, 5 aa, synthetic construct
EAAAR

SEQ ID NO: 21 - protein, 5 aa, synthetic construct
PAPAP

SEQ ID NO: 22 - protein, 12 aa, synthetic construct
AEAAAKEAAAKA

SEQ ID NO: 23 - protein, 51 aa, synthetic construct
ILTHDSSIRYLQEIYNSNNQKIVNLKEKVAQLEAQCQEPCKDTVQIHDITG

SEQ ID NO: 24 - skipped

SEQ ID NO: 25 - protein, 30 aa, synthetic construct
SLNGGGGSGGGGSGGGGSGGGGSGGGGSTS

SEQ ID NO: 26 - protein, 20 aa, synthetic construct
SGGSGGGGSGGGSGGGGSLQ

SEQ ID NO: 27 - protein, 26 aa, synthetic construct
SGGGSGGGGSGGGGSGGGGSGGGSLQ

SEQ ID NO: 28 - protein, 15 aa, synthetic construct
GGGGSGGGGSGGGGS

SEQ ID NO: 29 - protein, 493 aa, synthetic construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 486 aa, synthetic construct
MALPVTALLLPLALLLHAARPDIQMTQTTSSLSASLGDRVTISCRASQDISKYLNWYQQKPDGTVKLLIYHTSRLHSCVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKCLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 531 aa, synthetic construct
MDMRVPAQLLGLLLLWLRGARCDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKRGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSAKPTTTPAPRPPTPAPTIASQPLSLRPEAARPAAGGAVHTRGLDFAPRKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - protein, 19 aa, synthetic construct
CALGDSYGGGPLYTDKLIF

SEQ ID NO: 33 - protein, 14 aa, synthetic construct
CACDLLGYTDKLIF

SEQ ID NO: 34 - protein, 17 aa, synthetic construct
CACDALKRTDTDKLIFG

SEQ ID NO: 35 - protein, 15 aa, synthetic construct
CATWDRPEIYYKKLF

SEQ ID NO: 36 - protein, 15 aa, synthetic construct
CALWEEELGKKIKVF

SEQ ID NO: 37 - protein, 16 aa, synthetic construct
CALWEIQELGKKIKVF

SEQ ID NO: 38 - protein, 135 aa, synthetic construct
MERISSLIHLSLFWAGVMSAIELVPEHQTVPVSIGVPATLRCSMKGEAIGNYYINWYRKTQGNTMTFIYREKDIYGPGFKDNFQGDIDIAKNLAVLKILAPSERDEGSYYCACDLLGYTDKLIFGKGTRVTVEPR

SEQ ID NO: 39 - protein, 140 aa, synthetic construct
MVSLLHASTLAVLGALCVYGAGHLEQPQISSTKTLSKTARLECVVSGITISATSVYWYRERPGEVIQFLVSISYDGTVRKESGIPSGKFEVDRIPETSTSTLTIHNVEKQDIATYYCALWEEELGKKIKVFGPGTKLIIT

SEQ ID NO: 40 - protein, 137 aa, synthetic construct
MERISSLIHLSLFWAGVMSAIELVPEHQTVPVSIGVPATLRCSMKGEAIGNYYINWYRKTQGNTMTFIYREKDIYGPGFKDNFQGDIDIAKNLAVLKILAPSERDEGSYYCACDALKRTDTDKLIFGKGTRVTVEPR

SEQ ID NO: 41 - protein, 141 aa, synthetic construct
MVSLLHASTLAVLGALCVYGAGHLEQPQISSTKTLSKTARLECVVSGITISATSVYWYRERPGEVIQFLVSISYDGTVRKESGIPSGKFEVDRIPETSTSTLTIHNVEKQDIATYYCALWEIQELGKKIKVFGPGTKLIIT

SEQ ID NO: 42 - protein, 140 aa, synthetic construct
MVFSSLLCVFVAFSYSGSSVAQKVTQAQSSVSMPVRKAVTLNCLYETSWWSYYIFWYKQLPSKEMIFLIRQGSDEQNAKSGRYSVNFKKAAKSVALTISALQLEDSAKYFCALGDSYGGGPLYTDKLIFGKGTRVTVEPR

SEQ ID NO: 43 - protein, 136 aa, synthetic construct
MGWALLVLLAFLSPASQKSSNLEGGTKSVTRPTRSSAEITCDLTVINAFYIHWYLHQEGKAPQRLLYYDVSNSKDVLESGLSPGKYYTHTPRRWSWILILRNLIENDSGVYYCATWDRPEIYYKKLFGSGTTLVVT

SEQ ID NO: 44 - protein, 14 aa, synthetic construct
CATWDGPPYYKKLF

SEQ ID NO: 45 - protein, 11 aa, synthetic construct
CATWDGQKKLF

SEQ ID NO: 46 - protein, 11 aa, synthetic construct
CATWDNYKKLF

SEQ ID NO: 47 - protein, 18 aa, synthetic construct
CAASSPIRGYTGSDKLIF

SEQ ID NO: 48 - protein, 18 aa, synthetic construct
CAASSPIRGYTGSDKLIF

SEQ ID NO: 49 - protein, 17 aa, synthetic construct
CALGELRYWGIVDKLIF

SEQ ID NO: 50 - protein, 25 aa, synthetic construct
CALGELRGQISFLYLLGDTTDKLIF

SEQ ID NO: 51 - protein, 13 aa, synthetic construct
CATWDGFYYKKLF

SEQ ID NO: 52 - protein, 18 aa, synthetic construct
CAASSPIRGYTGSDKLIF

SEQ ID NO: 53 - protein, 144 aa, synthetic construct
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRGYTGSDKLIFGKGTRVTVEPR

SEQ ID NO: 54 - protein, 135 aa, synthetic construct
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGPPYYKKLFGSGTTLVVT

SEQ ID NO: 55 - protein, 138 aa, synthetic construct
MVFSSLLCVFVAFSYSGSSVAQKVTQAQSSVSMPVRKAVTLNCLYETSWWSYYIFWYKQLPSKEMIFLIRQGSDEQNAKSGRYSVNFKKAAKSVALTISALQLEDSAKYFCALGELRYWGIVDKLIFGKGTRVTVEPR

SEQ ID NO: 56 - protein, 132 aa, synthetic construct
MEWALAVLLAFLSPASQKSSNLEGRTKSVIRQTGSSAEITCDLAEGSNGYIHWYLHQEGKAPQRLQYYDSYNSKVVLESGVSPGKYYTYASTRNNLRLILRNLIENDSGVYYCATWDGQKKLFGSGTTLVVT

SEQ ID NO: 57 - protein, 146 aa, synthetic construct
MVFSSLLCVFVAFSYSGSSVAQKVTQAQSSVSMPVRKAVTLNCLYETSWWSYYIFWYKQLPSKEMIFLIRQGSDEQNAKSGRYSVNFKKAAKSVALTISALQLEDSAKYFCALGELRGQISFLYLLGDTTDKLIFGKGTRVTVEPR

SEQ ID NO: 58 - protein, 132 aa, synthetic construct
MVLALALLLAFLPPASQKSSNLEGRTKSVTRPTGSSAVITCDLPVENAVYTHWYLHQEGKAPQRLLYYDSYNSRVVLESGISREKYHTYASTGKSLKFILENLIERDSGVYYCATWDNYKKLFGSGTTLVVT

SEQ ID NO: 59 - protein, 144 aa, synthetic construct; identical to SEQ ID NO: 53

SEQ ID NO: 60 - protein, 132 aa, synthetic construct
MVLALALLLAFLPPASQKSSNLEGRTKSVTRPTGSSAVITCDLPVENAVYTHWYLHQEGKAPQRLLYYDSYNSRVVLESGISREKYHTYASTGKSLKFILENLIERDSGVYYCATWDNYMKLFGSGTTLVVT

SEQ ID NO: 61 - protein, 144 aa, synthetic construct; identical to SEQ ID NO: 53

SEQ ID NO: 62 - protein, 134 aa, synthetic construct
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGFYYKKLFGSGTTLVVT

SEQ ID NO: 63 - protein, 11 aa, synthetic construct
CATWDNYMKLF

SEQ ID NO: 64 - protein, 23 aa, synthetic construct
CASSYTLKLGDTPGRVRDWKLIF

SEQ ID NO: 65 - protein, 14 aa, synthetic construct
CATWDGPPYYKKLF

SEQ ID NO: 66 - protein, 26 aa, synthetic construct
CALGDYLGDKYPSYDLLGDTTDKLIF

SEQ ID NO: 67 - protein, 11 aa, synthetic construct
CATWDNYKKLF

SEQ ID NO: 68 - protein, 18 aa, synthetic construct
CAASSPIRGYTGSDKLIF

SEQ ID NO: 69 - protein, 11 aa, synthetic construct
CATWDNYKKLF

SEQ ID NO: 70 - protein, 144 aa, synthetic construct
MILTVGFSFLFFYRGTLCDKVTQSSPDQTVASGSEVVLLCTYDTVYSNPDLFWYRIRPDYSFQFVFYGDNSRSEGADFTQGRFSVKHILTQKAFHLVISPVRTEDSATYYCASSYTLKLGDTPGRVRDWKLIFGKGTRVTVEPR

SEQ ID NO: 71 - protein, 135 aa, synthetic construct; identical to SEQ ID NO: 54

SEQ ID NO: 72 - protein, 147 aa, synthetic construct
MVFSSLLCVFVAFSYSGSSVAQKVTQAQSSVSMPVRKAVTLNCLYETSWWSYYIFWYKQLPSKEMIFLIRQGSDEQNAKSGRYSVNFKKAAKSVALTISALQLEDSAKYFCALGDYLGDKYPSYDLLGDTTDKLIFGKGTRVTVEPR

SEQ ID NO: 73 - protein, 132 aa, synthetic construct; identical to SEQ ID NO: 58

SEQ ID NO: 74 - protein, 144 aa, synthetic construct; identical to SEQ ID NO: 53

SEQ ID NO: 75 - protein, 132 aa, synthetic construct; identical to SEQ ID NO: 58

SEQ ID NO: 76 - protein, 20 aa, synthetic construct
CALGNGNHIGYWRYTDKLIF

SEQ ID NO: 77 - protein, 13 aa, synthetic construct
CATWDRLYYKKLF

SEQ ID NO: 78 - protein, 141 aa, synthetic construct
MVFSSLLCVFVAFSYSGSSVAQKVTQAQSSVSMPVRKAVTLNCLYETSWWSYYIFWYKQLPSKEMIFLIRQGSDEQNAKSGRYSVNFKKAAKSVALTISALQLEDSAKYFCALGNGNHIGYWRYTDKLIFGKGTRVTVEPR

SEQ ID NO: 79 - protein, 134 aa, synthetic construct
MVWALLVLLAFLSPASQKSSNLEGGTKSVTRPTRSSAEITCDLTVINAFYIHWYLHQEGKAPQRLLYYDVSNSKDVLESGLSPGKYYTHTPRRWSWILILRNLIENDSGVYYCATWDRLYYKKLFGSGTTLVVT

SEQ ID NO: 80 - protein, 10 aa, synthetic construct
CATWDSSKLF

SEQ ID NO: 81 - protein, 17 aa, synthetic construct
CAFTGLGDTSHADKLIF

SEQ ID NO: 82 - protein, 131 aa, synthetic construct
MLLALALLLAFLPPASQKSSNLEGRTKSVTRPTGSSAVITCDLPVENAVYTHWYLHQEGKAPQRLLYYDSYNSRVVLESGISREKYHTYASTGKSLKFILENLIERDSGVYYCATWDSSKLFGSGTTLVVT

SEQ ID NO: 83 - protein, 138 aa, synthetic construct
MILTVGFSFLFFYRGTLCDKVTQSSPDQTVASGSEVVLLCTYDTVYSNPDLFWYRIRPDYSFQFVFYGDNSRSEGADFTQGRFSVKHILTQKAFHLVISPVRTEDSATYYCAFTGLGDTSHADKLIFGKGTRVTVEPR

SEQ ID NO: 84 - protein, 138 aa, synthetic construct; identical to SEQ ID NO: 83

SEQ ID NO: 85 - protein, 21 aa, synthetic construct
CAFIVYDFLGELGEDTDKLIF

SEQ ID NO: 86 - protein, 15 aa, synthetic construct
CATWDSKPHYYKKLF

SEQ ID NO: 87 - protein, 251 aa, synthetic construct
MRWALLVLLAFLSPASQKSSNLEGRTKSVTRQTGSSAEITCDLTVTNTFYIHWYLHQEGKAPQRLLYYDVSTARDVLESGLSPGKYYTHTPRRWSWILRLQNLIENDSGVYYCATWDSKPHYYKKLFGSGTTLVVTDKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTV

SEQ ID NO: 88 - protein, 15 aa, synthetic construct
CACGLPRSLADKLIF

SEQ ID NO: 89 - protein, 136 aa, synthetic construct
MILTVGFSFLFFYRGTLCDKVTQSSPDQTVASGSEVVLLCTYDTVYSNPDLFWYRIRPDYSFQFVFYGDNSRSEGADFTQGRFSVKHILTQKAFHLVISPVRTEDSATYYCACGLPRSLADKLIFGKGTRVTVEPR

SEQ ID NO: 90 - protein, 11 aa, synthetic construct
CATWDLHKKLF

SEQ ID NO: 91 - protein, 247 aa, synthetic construct
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDLHKKLFGSGTTLVVTDKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTV

SEQ ID NO: 92 - protein, 17 aa, synthetic construct
CALGEFPYWGIPDKLIF

SEQ ID NO: 93 - protein, 138 aa, synthetic construct
MVFSSLLCVFVAFSYSGSSVAQKVTQAQSSVSMPVRKAVTLNCLYETSWWSYYIFWYKQLPSKEMIFLIRQGSDEQNAKSGRYSVNFKKAAKSVALTISALQLEDSAKYFCALGEFPYWGIPDKLIFGKGTRVTVEPR

SEQ ID NO: 94 - protein, 12 aa, synthetic construct
CATWDGYYKKLF

SEQ ID NO: 95 - protein, 248 aa, synthetic construct
MRWALLVLLAFLSPASQKSSNLEGRTKSVTRQTGSSAEITCDLTVTNTFYIHWYLHQEGKAPQRLLYYDVSTARDVLESGLSPGKYYTHTPRRWSWILRLQNLIENDSGVYYCATWDGYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITM

SEQ ID NO: 96 - protein, 25 aa, synthetic construct
CALGGLGWGYAGDEGGGKLTDKLIF

SEQ ID NO: 97 - protein, 146 aa, synthetic construct
MVFSSLLCVFVAFSYSGSSVAQKVTQAQSSVSMPVRKAVTLNCLYETSWWSYYIFWYKQLPSKEMIFLIRQGSDEQNAKSGRYSVNFKKAAKSVALTISALQLEDSAKYFCALGGLGWGYAGDEGGGKLTDKLIFGKGTRVTVEPR

SEQ ID NO: 98 - protein, 14 aa, synthetic construct
CALWEVHPNYKKLF

SEQ ID NO: 99 - protein, 254 aa, synthetic construct
MVSLLHASTLAVLGALCVYGAGHLEQPQISSTKTLSKTARLECVVSGITISATSVYWYRERPGEVIQFLVSISYDGTVRKESGIPSGKFEVDRIPETSTSTLTIHNVEKQDIATYYCALWEVHPNYKKLFGSGTTLVVTDKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTV

SEQ ID NO: 100 - protein, 153 aa, synthetic construct
SQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 101 - protein, 173 aa, synthetic construct
DKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 102 - protein, 189 aa, synthetic construct
DKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKYNYSKDANDVITMDPKDNWSKDANDTLLLQLTNTSAYYTYLLLLLKSVVYFAIITCCLLRRTAFCCNGEKS

SEQ ID NO: 103 - DNA, 367 bp, synthetic construct
  source 1..367: mol_type = other DNA
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgtttctgctccccgagctcaataaaagagcccacaacccctcact

SEQ ID NO: 104 - DNA, 594 bp, synthetic construct
  source 1..594: mol_type = other DNA
aatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatt

SEQ ID NO: 105 - DNA, 1182 bp, synthetic construct
  source 1..1182: mol_type = other DNA
gctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcttgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 106 - DNA, 399 bp, synthetic construct
  source 1..399: mol_type = other DNA
tttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagccca

SEQ ID NO: 107 - protein, 19 aa, synthetic construct
ATNFSLLKQAGDVEENPGP

SEQ ID NO: 108 - DNA, 57 bp, synthetic construct
  source 1..57: mol_type = other DNA
gccaccaatttcagcctgctgaaacaggctggcgacgtggaagagaaccctgggccc

SEQ ID NO: 109 - protein, 18 aa, synthetic construct
EGRGSLLTCGDVEENPGP

SEQ ID NO: 110 - DNA, 54 bp, synthetic construct
  source 1..54: mol_type = other DNA
gaaggccgcggcagcctgctgacctgcggagacgtggaagaaaaccctggcccg

SEQ ID NO: 111 - DNA, 3414 bp, synthetic construct
  source 1..3414: mol_type = other DNA
atggtgtccctgctgcacgccagcaccctggccgtgctgggcgccctgtgcgtgtatggcgccggacacctggaacagccccagatcagcagcaccaagaccctgagcaagaccgccaggctggaatgcgtggtgtccggcatcaccatcagcgccacctccgtgtactggtacagagagagacccggcgaggtcatccagttcctggtgtccatcagctacgacggcaccgtgcggaaagagagcggcatccccagcggcaagttcgaggtggacagaatccccgagaccagcacctccaccctgaccatccacaacgtggagaagcaggacatcgccacctactactgcgccctgtgggagatccaggaactgggcaagaaaatcaaggtgttcggccctggcaccaagctgatcatcaccgacaagcagctggacgccgacgtgagccccaagcctaccatcttcctgcccagcatcgccgagaccaagctgcagaaggccggcacctacctgtgcctgctggaaaagttcttccccgacgtgatcaagatccactgggaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggaccccaaggacaactgcagcaaggacgccaacgataccctgctgctgcagctgaccaacaccagcgcctactacatgtatctcctgctgctgctgaagagcgtggtgtacttcgccatcatcacctgctgtctcctgaggcggaccgccttctgctgcaacggcgagaagagcggatctggcgaaggccgcggcagcctgctgacctgcggcgacgtggaagagaaccctgggcccatggcactccccgtcaccgcccttctcttgcccctcgccctgctgctgcatgctgccaggcccgacattgtgctcactcagtcacctcccagcctggccatgagcctgggaaaaagggccaccatctcctgtagagccagtgagtccgtcacaatcttggggagccatcttattcactggtatcagcagaagcccgggcagcctccaacccttcttattcagctcgcgtcaaacgtccagacgggtgtacctgccagattttctggtagcgggtcccgcactgattttacactgaccatagatccagtggaagaagacgatgtggccgtgtattattgtctgcagagcagaacgattcctcgcacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcaccgactacagcataaattgggtgaaacgggcccctggaaagggcctcaaatggatgggttggatcaataccgaaactagggagcctgcttatgcatatgacttccgcgggagattcgccttttcactcgagacatctgcctctactgcttacctccaaataaacaacctcaagtatgaagatacagccacttacttttgcgccctcgactatagttacgcgatggactactggggacagggaactagtgttaccgtcagttccacaacaacccctgcccccagacctcctaccccagcccctacaattgccagccagcctctgagcctgagacccgaggcttgtagaccagctgctggcggagccgtgcacaccagaggactggatttcgcctgcgacatctacatctgggcccctctggccggcacatgtggcgtgctgctgctgagcctcgtgatcaccctgtactgcaagcggggcagaaagaaactgctgtacatctttaagcagcccttcatgcggcccgtgcagaccacccaggaagaggacggctgctcctgcagattccccgaggaagaagaaggcggctgcgagctgagagtgaagttcagcagatccgccgacgcccctgcctacaagcagggacagaaccagctgtacaacgagctgaacctgggcagacgggaagagtacgacgtgctggacaagcggagaggccgggaccctgaaatgggcggcaagcccagaagaaagaacccccaggaaggcctgtataacgaactgcagaaagacaagatggccgaggcctacagcgagatcggaatgaagggcgagcggagaagaggcaagggccacgatggcctgtaccagggcctgagcaccgccaccaaggacacctatgacgccctgcacatgcaggccctgcctccaagaggcagcggcgccaccaatttcagcctgctgaaacaggctggagacgtggaagaaaaccctggcccgatggagcggatcagcagcctgatccacctgagcctgttctgggccggagtgatgagcgccatcgagctggtgcccgagcaccagaccgtgcccgtgagcatcggcgtgcccgccaccctgcggtgcagcatgaagggcgaggccatcggcaactactacatcaactggtacagaaagacccagggcaacaccatgaccttcatctaccgggagaaggacatctacggccctggcttcaaggacaacttccagggcgacatcgacatcgccaagaacctggccgtgctgaagatcctggcccccagcgagagggacgagggcagctactactgcgcctgcgacgccctgaagagaaccgacaccgacaagctgatcttcggcaagggcacccgggtgaccgtggagcccagaagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctgtga

SEQ ID NO: 112 - protein, 1137 aa, synthetic construct
MVSLLHASTLAVLGALCVYGAGHLEQPQISSTKTLSKTARLECVVSGITISATSVYWYRERPGEVIQFLVSISYDGTVRKESGIPSGKFEVDRIPETSTSTLTIHNVEKQDIATYYCALWEIQELGKKIKVFGPGTKLIITDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKSGSGEGRGSLLTCGDVEENPGPMALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMERISSLIHLSLFWAGVMSAIELVPEHQTVPVSIGVPATLRCSMKGEAIGNYYINWYRKTQGNTMTFIYREKDIYGPGFKDNFQGDIDIAKNLAVLKILAPSERDEGSYYCACDALKRTDTDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 113 - DNA, 2253 bp, synthetic construct
  source 1..2253: mol_type = other DNA
atggcactccccgtcaccgcccttctcttgcccctcgccctgctgctgcatgctgccaggcccgacattgtgctcactcagtcacctcccagcctggccatgagcctgggaaaaagggccaccatctcctgtagagccagtgagtccgtcacaatcttggggagccatcttattcactggtatcagcagaagcccgggcagcctccaacccttcttattcagctcgcgtcaaacgtccagacgggtgtacctgccagattttctggtagcgggtcccgcactgattttacactgaccatagatccagtggaagaagacgatgtggccgtgtattattgtctgcagagcagaacgattcctcgcacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcaccgactacagcataaattgggtgaaacgggcccctggaaagggcctcaaatggatgggttggatcaataccgaaactagggagcctgcttatgcatatgacttccgcgggagattcgccttttcactcgagacatctgcctctactgcttacctccaaataaacaacctcaagtatgaagatacagccacttacttttgcgccctcgactatagttacgcgatggactactggggacagggaacctccgttaccgtcagttccacaacaacccctgcccccagacctcctaccccagcccctacaattgccagccagcctctgagcctgaggcccgaggcttgtagaccagctgctggcggagccgtgcacaccagaggactggatttcgcctgcgacatctacatctgggcccctctggccggcacatgtggcgtgctgctgctgagcctcgtgatcaccctgtactgcaagcggggcagaaagaaactgctgtacatctttaagcagcccttcatgcggcccgtgcagaccacccaggaagaggacggctgctcctgcagattccccgaggaagaagaaggcggctgcgagctgagagtgaagttcagcagatccgccgacgcccctgcctacaagcagggacagaaccagctgtacaacgagctgaacctgggcagacgggaagagtacgacgtgctggacaagcggagaggccgggaccctgaaatgggcggcaagcccagaagaaagaacccccaggaaggcctgtataacgaactgcagaaagacaagatggccgaggcctacagcgagatcggaatgaagggcgagcggagaagaggcaagggccacgatggcctgtaccagggcctgagcaccgccaccaaggacacctatgacgccctgcacatgcaggccctgcctccaagaggcagcggcgaaggccgcggcagcctgctgacctgcggcgatgtggaagaaaaccctgggcccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 114 - protein, 750 aa, synthetic construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 115 - DNA, 2307 bp, synthetic construct
  source 1..2307: mol_type = other DNA
atggaatatgccagtgacgcatctctcgatcccgaagccccttggccaccggcgccaagagctagagcttgcagagtgttgccttgggctttggtggccggactgctgctcctgctgctgctggctgctgcctgtgccgtgtttctcgcatgtccttgggctgtgtccggtgcaagagcctctcctggatctgccgcgagtccgcgcctgcgagagggaccagaattgtcaccggatgaccctgcaggccttctcgatctcaggcaaggaatgtttgcgcagctggtggcacaaaacgtgttgcttatagacggaccacttagctggtattctgatcccggcctggccggggtttcattgaccggcggcctttcctacaaggaagataccaaggaactggtggtcgccaaggccggagtatactatgttttcttccaattggagttgcggcgcgtcgtggcaggggaagggagcggttctgtctctttggctctccacctgcaacccttgagatccgcggctggagctgcagctttggcgctcactgttgacctgccacctgcaagttccgaggctcggaactcagcgttcgggtttcaaggcagactgctgcacctgagcgccgggcaaagacttggtgtacacttgcacactgaggctagagcccgccacgcttggcaactcacacaaggggctacagtgcttggcctgttccgagtaacgcccgaaattccagcgggcctgccaagtcctagatctgaaggcagcggcgaaggccgcggcagcctgctgacctgcggagacgtggaagaaaaccctggcccgatggcactccccgtcaccgcccttctcttgcccctcgccctgctgctgcatgctgccaggcccgacattgtgctcactcagtcacctcccagcctggccatgagcctgggaaaaagggccaccatctcctgtagagccagtgagtccgtcacaatcttggggagccatcttattcactggtatcagcagaagcccgggcagcctccaacccttcttattcagctcgcgtcaaacgtccagacgggtgtacctgccagattttctggtagcgggtcccgcactgattttacactgaccatagatccagtggaagaagacgatgtggccgtgtattattgtctgcagagcagaacgattcctcgcacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcaccgactacagcataaattgggtgaaacgggcccctggaaagggcctcaaatggatgggttggatcaataccgaaactagggagcctgcttatgcatatgacttccgcgggagattcgccttttcactcgagacatctgcctctactgcttacctccaaataaacaacctcaagtatgaagatacagccacttacttttgcgccctcgactatagttacgcgatggactactggggacagggaacctccgttaccgtcagttccaccactactccagccccgaggcccccaacaccagctcctactatcgcatctcagccacttagtcttagacctgaggcatgcaggcccgccgccgggggtgccgtgcatacccgcggactggactttgcgtgtgacatctatatttgggcccctctggccggtacttgcggtgttcttttgctgtcactggttattactctctattgcaaccatagaaacagatcaaagaggtccagactgctgcactcggactacatgaacatgaccccccgcaggcccggccccaccagaaaacactatcagccttacgccccccccagggattttgctgcctatagatctcgcgtgaagtttagcagaagcgcagacgctcccgcatatcaacagggtcaaaatcagctatataatgagctgaatcttggccggcgagaggaatacgatgtactggacaaaagacgggggcgagatcccgagatgggaggaaaacctcgacgtaaaaatcctcaagagggtctgtataacgagctgcagaaagacaaaatggcggaggcgtacagcgaaatagggatgaagggcgagcgtcgacgaggtaagggacatgatggactgtatcaaggactgtctacggccacaaaggatacatacgacgctctccacatgcaggctctgccacctcggtga

SEQ ID NO: 116 - protein, 768 aa, synthetic construct
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSEGSGEGRGSLLTCGDVEENPGPMALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPAAGGAVHTRGLDFACDIYIWAPLAGTCGVLLLSLVITLYCNHRNRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 117 - DNA, 22 bp, synthetic construct
  source 1..22: mol_type = other DNA
tcctggttgctgtctctttatg

SEQ ID NO: 118 - DNA, 18 bp, synthetic construct
  source 1..18: mol_type = other DNA
tgacaggtggtggcaatg

SEQ ID NO: 119 - DNA, 23 bp, synthetic construct
  source 1..23: mol_type = other DNA
  misc_feature 23: note = 6-FAM (Fluorescein) modification
tgcactgtgtttgctgacgcaac

SEQ ID NO: 120 - DNA, 22 bp, synthetic construct
  source 1..22: mol_type = other DNA
gctgtcatctcttgtgggctgt

SEQ ID NO: 121 - DNA, 21 bp, synthetic construct
  source 1..21: mol_type = other DNA
actcatgggagctgctggttc

SEQ ID NO: 122 - DNA, 26 bp, synthetic construct
  source 1..26: mol_type = other DNA
  misc_feature 26: note = VIC modification
cctgtcatgcccacacaaatctctcc

SEQ ID NO: 123 - protein, 19 aa, synthetic construct
CALGDPYGWGIRNTDKLIF

SEQ ID NO: 124 - protein, 140 aa, synthetic construct
MVFSSLLCVFVAFSYSGSSVAQKVTQAQSSVSMPVRKAVTLNCLYETSWWSYYIFWYKQLPSKEMIFLIRQGSDEQNAKSGRYSVNFKKAAKSVALTISALQLEDSAKYFCALGDPYGWGIRNTDKLIFGKGTRVTVEPR

SEQ ID NO: 125 - protein, 15 aa, synthetic construct
CATWDRPPTGWFKIF

SEQ ID NO: 126 - protein, 138 aa, synthetic construct
MRWALLVLLAFLSPASQKSSNLEGGTKSVTRPTRSSAEITCDLTVINAFYIHWYLHQEGKAPQRLLYYDVSNSKDVLESGLSPGKYYTHTPRRWSWILILRNLIENDSGVYYCATWDRPPTGWFKIFAEGTKLIVTSP

SEQ ID NO: 127 - protein, 20 aa, synthetic construct
CALGELAFLLLTGGSDKLIF

SEQ ID NO: 128 - protein, 141 aa, synthetic construct
MVFSSLLCVFVAFSYSGSSVAQKVTQAQSSVSMPVRKAVTLNCLYETSWWSYYIFWYKQLPSKEMIFLIRQGSDEQNAKSGRYSVNFKKAAKSVALTISALQLEDSAKYFCALGELAFLLLTGGSDKLIFGKGTRVTVEPR

SEQ ID NO: 129 - protein, 16 aa, synthetic construct
CATWVNQRGSDWIKTF

SEQ ID NO: 130 - protein, 139 aa, synthetic construct
MVLALALLLAFLPPASQKSSNLEGRTKSVTRPTGSSAVITCDLPVENAVYTHWYLHQEGKAPQRLLYYDSYNSRVVLESGISREKYHTYASTGKSLKFILENLIERDSGVYYCATWVNQRGSDWIKTFAKGTRLIVTSP

SEQ ID NO: 131 - protein, 26 aa, synthetic construct
CAFPPSDWDWGVPWSRGALYTDKLIF

SEQ ID NO: 132 - protein, 147 aa, synthetic construct
MILTVGFSFLFFYRGTLCDKVTQSSPDQTVASGSEVVLLCTYDTVYSNPDLFWYRIRPDYSFQFVFYGDNSRSEGADFTQGRFSVKHILTQKAFHLVISPVRTEDSATYYCAFPPSDWDWGVPWSRGALYTDKLIFGKGTRVTVEPR

SEQ ID NO: 133 - protein, 15 aa, synthetic construct
CATWDGHPNYYKKLF

SEQ ID NO: 134 - protein, 136 aa, synthetic construct
MQWALAVLLAFLSPASQKSSNLEGRTKSVIRQTGSSAEITCDLAEGSNGYIHWYLHQEGKAPQRLQYYDSYNSKVVLESGVSPGKYYTYASTRNNLRLILRNLIENDSGVYYCATWDGHPNYYKKLFGSGTTLVVT

SEQ ID NO: 135 - protein, 21 aa, synthetic construct
CALGELPLSLTQNWGEYKLIF

SEQ ID NO: 136 - protein, 142 aa, synthetic construct
MVFSSLLCVFVAFSYSGSSVAQKVTQAQSSVSMPVRKAVTLNCLYETSWWSYYIFWYKQLPSKEMIFLIRQGSDEQNAKSGRYSVNFKKAAKSVALTISALQLEDSAKYFCALGELPLSLTQNWGEYKLIFGKGTRVTVEPR

SEQ ID NO: 137 - protein, 13 aa, synthetic construct
CATWDLNYYKKLF

SEQ ID NO: 138 - protein, 134 aa, synthetic construct
MQWALAVLLAFLSPASQKSSNLEGRTKSVIRQTGSSAEITCDLAEGSNGYIHWYLHQEGKAPQRLQYYDSYNSKVVLESGVSPGKYYTYASTRNNLRLILRNLIENDSGVYYCATWDLNYYKKLFGSGTTLVVT

SEQ ID NO: 139 - protein, 16 aa, synthetic construct
CALGEPLPTIWDKLIF

SEQ ID NO: 140 - protein, 137 aa, synthetic construct
MVFSSLLCVFVAFSYSGSSVAQKVTQAQSSVSMPVRKAVTLNCLYETSWWSYYIFWYKQLPSKEMIFLIRQGSDEQNAKSGRYSVNFKKAAKSVALTISALQLEDSAKYFCALGEPLPTIWDKLIFGKGTRVTVEPR

SEQ ID NO: 141 - protein, 15 aa, synthetic construct
CATWDRRQNYYKKLF

SEQ ID NO: 142 - protein, 136 aa, synthetic construct
MRWALLVLLAFLSPASQKSSNLEGRTKSVTRQTGSSAEITCDLTVTNTFYIHWYLHQEGKAPQRLLYYDVSTARDVLESGLSPGKYYTHTPRRWSWILRLQNLIENDSGVYYCATWDRRQNYYKKLFGSGTTLVVT

SEQ ID NO: 143 - protein, 22 aa, synthetic construct
CALGESLPSLAGYPPVPDKLIF

SEQ ID NO: 144 - protein, 143 aa, synthetic construct
MVFSSLLCVFVAFSYSGSSVAQKVTQAQSSVSMPVRKAVTLNCLYETSWWSYYIFWYKQLPSKEMIFLIRQGSDEQNAKSGRYSVNFKKAAKSVALTISALQLEDSAKYFCALGESLPSLAGYPPVPDKLIFGKGTRVTVEPR

SEQ ID NO: 145 - protein, 12 aa, synthetic construct
CATWDRPWIKTF

SEQ ID NO: 146 - protein, 135 aa, synthetic construct
MRWALLVLLAFLSPASQKSSNLEGRTKSVTRQTGSSAEITCDLTVTNTFYIHWYLHQEGKAPQRLLYYDVSTARDVLESGLSPGKYYTHTPRRWSWILRLQNLIENDSGVYYCATWDRPWIKTFAKGTRLIVTSP

SEQ ID NO: 147 - protein, 17 aa, synthetic construct
CALGAPGIRLITDKLIF

SEQ ID NO: 148 - protein, 138 aa, synthetic construct
MVFSSLLCVFVAFSYSGSSVAQKVTQAQSSVSMPVRKAVTLNCLYETSWWSYYIFWYKQLPSKEMIFLIRQGSDEQNAKSGRYSVNFKKAAKSVALTISALQLEDSAKYFCALGAPGIRLITDKLIFGKGTRVTVEPR

SEQ ID NO: 149 - protein, 11 aa, synthetic construct
CATWDFYKKLF

SEQ ID NO: 150 - protein, 132 aa, synthetic construct
MQWALAVLLAFLSPASQKSSNLEGRTKSVIRQTGSSAEITCDLAEGSNGYIHWYLHQEGKAPQRLQYYDSYNSKVVLESGVSPGKYYTYASTRNNLRLILRNLIENDSGVYYCATWDFYKKLFGSGTTLVVT

SEQ ID NO: 151 - DNA, 3411 bp, synthetic construct
  source 1..3411: mol_type = other DNA
atggtgtccctgctgcacgccagcaccctggccgtgctgggcgccctgtgcgtgtatggcgccggacacctggaacagccccagatcagcagcaccaagaccctgagcaagaccgccaggctggaatgcgtggtgtccggcatcaccatcagcgccacctccgtgtactggtacagagagagacccggcgaggtcatccagttcctggtgtccatcagctacgacggcaccgtgcggaaagagagcggcatccccagcggcaagttcgaggtggacagaatccccgagaccagcacctccaccctgaccatccacaacgtggagaagcaggacatcgccacctactactgcgccctgtgggagatccaggaactgggcaagaaaatcaaggtgttcggccctggcaccaagctgatcatcaccgacaagcagctggacgccgacgtgagccccaagcctaccatcttcctgcccagcatcgccgagaccaagctgcagaaggccggcacctacctgtgcctgctggaaaagttcttccccgacgtgatcaagatccactgggaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggaccccaaggacaactgcagcaaggacgccaacgataccctgctgctgcagctgaccaacaccagcgcctactacatgtatctcctgctgctgctgaagagcgtggtgtacttcgccatcatcacctgctgtctcctgaggcggaccgccttctgctgcaacggcgagaagagcggatctggcgccaccaatttcagcctgctgaaacaggctggagacgtggaagagaaccctgggcccatggcactccccgtcaccgcccttctcttgcccctcgccctgctgctgcatgctgccaggcccgacattgtgctcactcagtcacctcccagcctggccatgagcctgggaaaaagggccaccatctcctgtagagccagtgagtccgtcacaatcttggggagccatcttattcactggtatcagcagaagcccgggcagcctccaacccttcttattcagctcgcgtcaaacgtccagacgggtgtacctgccagattttctggtagcgggtcccgcactgattttacactgaccatagatccagtggaagaagacgatgtggccgtgtattattgtctgcagagcagaacgattcctcgcacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcaccgactacagcataaattgggtgaaacgggcccctggaaagggcctcaaatggatgggttggatcaataccgaaactagggagcctgcttatgcatatgacttccgcgggagattcgccttttcactcgagacatctgcctctactgcttacctccaaataaacaacctcaagtatgaagatacagccacttacttttgcgccctcgactatagttacgcgatggactactggggacagggaactagtgttaccgtcagttccacaacaacccctgcccccagacctcctaccccagcccctacaattgccagccagcctctgagcctgagacccgaggcttgtagaccagctgctggcggagccgtgcacaccagaggactggatttcgcctgcgacatctacatctgggcccctctggccggcacatgtggcgtgctgctgctgagcctcgtgatcaccctgtactgcaagcggggcagaaagaaactgctgtacatctttaagcagcccttcatgcggcccgtgcagaccacccaggaagaggacggctgctcctgcagattccccgaggaagaagaaggcggctgcgagctgagagtgaagttcagcagatccgccgacgcccctgcctacaagcagggacagaaccagctgtacaacgagctgaacctgggcagacgggaagagtacgacgtgctggacaagcggagaggccgggaccctgaaatgggcggcaagcccagaagaaagaacccccaggaaggcctgtataacgaactgcagaaagacaagatggccgaggcctacagcgagatcggaatgaagggcgagcggagaagaggcaagggccacgatggcctgtaccagggcctgagcaccgccaccaaggacacctatgacgccctgcacatgcaggccctgcctccaagaggcagcggcgaaggccgcggcagcctgctgacctgcggagacgtggaagaaaaccctggcccgatggagcggatcagcagcctgatccacctgagcctgttctgggccggagtgatgagcgccatcgagctggtgcccgagcaccagaccgtgcccgtgagcatcggcgtgcccgccaccctgcggtgcagcatgaagggcgaggccatcggcaactactacatcaactggtacagaaagacccagggcaacaccatgaccttcatctaccgggagaaggacatctacggccctggcttcaaggacaacttccagggcgacatcgacatcgccaagaacctggccgtgctgaagatcctggcccccagcgagagggacgagggcagctactactgcgcctgcgacgccctgaagagaaccgacaccgacaagctgatcttcggcaagggcacccgggtgaccgtggagcccagaagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctg

SEQ ID NO: 152 - protein, 1137 aa, synthetic construct
MVSLLHASTLAVLGALCVYGAGHLEQPQISSTKTLSKTARLECVVSGITISATSVYWYRERPGEVIQFLVSISYDGTVRKESGIPSGKFEVDRIPETSTSTLTIHNVEKQDIATYYCALWEIQELGKKIKVFGPGTKLIITDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKSGSGATNFSLLKQAGDVEENPGPMALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMERISSLIHLSLFWAGVMSAIELVPEHQTVPVSIGVPATLRCSMKGEAIGNYYINWYRKTQGNTMTFIYREKDIYGPGFKDNFQGDIDIAKNLAVLKILAPSERDEGSYYCACDALKRTDTDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 153 - DNA, 2253 bp, synthetic construct
  source 1..2253: mol_type = other DNA
atggcactccccgtcaccgcccttctcttgcccctcgccctgctgctgcatgctgccaggcccgacattgtgctcactcagtcacctcccagcctggccatgagcctgggaaaaagggccaccatctcctgtagagccagtgagtccgtcacaatcttggggagccatcttattcactggtatcagcagaagcccgggcagcctccaacccttcttattcagctcgcgtcaaacgtccagacgggtgtacctgccagattttctggtagcgggtcccgcactgattttacactgaccatagatccagtggaagaagacgatgtggccgtgtattattgtctgcagagcagaacgattcctcgcacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcaccgactacagcataaattgggtgaaacgggcccctggaaagggcctcaaatggatgggttggatcaataccgaaactagggagcctgcttatgcatatgacttccgcgggagattcgccttttcactcgagacatctgcctctactgcttacctccaaataaacaacctcaagtatgaagatacagccacttacttttgcgccctcgactatagttacgcgatggactactggggacagggaactagtgttaccgtcagttccacaacaacccctgcccccagacctcctaccccagcccctacaattgccagccagcctctgagcctgagacccgaggcttgtagaccagctgctggcggagccgtgcacaccagaggactggatttcgcctgcgacatctacatctgggcccctctggccggcacatgtggcgtgctgctgctgagcctcgtgatcaccctgtactgcaagcggggcagaaagaaactgctgtacatctttaagcagcccttcatgcggcccgtgcagaccacccaggaagaggacggctgctcctgcagattccccgaggaagaagaaggcggctgcgagctgagagtgaagttcagcagatccgccgacgcccctgcctacaagcagggacagaaccagctgtacaacgagctgaacctgggcagacgggaagagtacgacgtgctggacaagcggagaggccgggaccctgaaatgggcggcaagcccagaagaaagaacccccaggaaggcctgtataacgaactgcagaaagacaagatggccgaggcctacagcgagatcggaatgaagggcgagcggagaagaggcaagggccacgatggcctgtaccagggcctgagcaccgccaccaaggacacctatgacgccctgcacatgcaggccctgcctccaagaggcagcggcgccaccaatttcagcctgctgaaacaggctggagacgtggaagaaaaccctgggcccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 154 - protein, 751 aa, synthetic construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 155 - DNA, 2658 bp, synthetic construct
  source 1..2658: mol_type = other DNA
atggatatgcgggtaccagcgcaactgcttgggctgcttcttttgtggctgagaggtgctcggtgtgacattcaattgacccagtctccttcttccttgagcgctagcgtcggcgaccgcgtcaccatcacatgcaaagcctcccaggatgtgggaacgtcagtagcctggtaccagcaaaagcctggaaaagcacctaagcttttgatctattggacctctactaggcatacaggggtcccgagtcgcttttccggctccggtagcggcacagatttcaccttcacaatatcttctttacaaccagaagacatcgccacttactactgtcagcagtattctttgtaccgttcctttgggcaaggaacaaaagtagaaataaagagaggtggctctggaagcggcgggtccgggagtggtgggtctggttctgaggtgcagctcgtggagtcaggcggcggggtcgtccagccagggagatctctgaggctgagctgcagcgcgtcggggtttgactttaccacctattggatgagttgggtgcgccaggcccctgggaagggacttgaatggataggcgagatccatcccgacagctccacaatcaattacgcaccttccttgaaagacaggtttacaatctcgagagataacgcaaagaacacactgtttttgcaaatggactcactccgccctgaggacaccggagtgtacttctgcgcgagcctatattttgggttcccctggttcgcatactggggccagggaacccccgtaactgtctcaagcgcaaagccaaccactaccccagcgccgcgaccaccaacaccggcgcccaccatcgcgtcgcagcccctgtctttgcgacctgaggctgctaggcctgccgctggcggagctgtgcacaccagggggttggatttcgcccctcggaagattgaagttatgtatcccccaccctacctcgacaacgaaaaatccaacgggacaataattcacgtaaagggcaaacacctgtgtcccagtccattattccccgggcccagcaaacccttctgggtgctggtggtggtcggtggggtgctggcctgttattccttgctagttaccgtcgccttcattatattctgggtaagatctaagcgttcacgccttctccactctgattacatgaacatgaccccaagaaggcccgggcctaccaggaagcactatcagccgtatgccccacctcgcgacttcgctgcctacaggtctagggtaaaattctctcgcagtgcagagcctcccgcttatcagcaggggcaaaatcaattgtacaatgagctgaacctcggacggcgcgaagagtacgatgtgctggataagcgtcgcgggagagaccccgagatgggaggcaaaccccgcagaaagaatccccaggagggactctataacgagttgcagaaggacaaaatggccgaggcctactcggaaatcggaatgaaaggggaaagaaggcgcggtaaaggccacgacggtctctatcagggcttatctacagctacaaaggatacttacgatgcgctgcacatgcaggcactgccccctcggggatctggcgccaccaatttcagcctgctgaaacaggctggcgacgtggaagagaaccctgggcccatgccaccgcctaggctgctcttctttctcttgttcctgacacctatggaggtcagacccgaagagcctttggtggttaaagtggaggagggagacaacgccgtgctgcagtgtttgaaaggaacatccgacgggcctacccagcaactgacttggtccagagagtctcccctgaaaccattcctgaaactaagcctcgggcttcctggtctgggcatccatatgcgacctttagccatctggctctttattttcaatgtctcacagcagatgggaggcttttacctgtgccagccaggcccaccaagtgaaaaagcctggcagccagggtggaccgttaatgttgaggggtctggcgagcttttccggtggaacgtctctgacctcgggggcttggggtgcggtctgaagaaccgatcgtctgagggaccttcttcacccagcggcaagttgatgagccccaagttgtatgtctgggctaaggaccgaccggaaatctgggagggagagcctccctgccttccccctagggatagccttaaccagagcctctcccaagacttgacaatggcaccaggcagcaccttatggctctcatgcggggtaccaccagactcagtgtcacgaggaccactgagctggactcatgtgcacccaaagggccccaaatctctgctgagtctggaactgaaagacgaccgacctgctagggatatgtgggtgatggaaaccggcctgcttctcccacgcgccacagctcaagatgcaggtaaatattattgtcacagaggaaatctcacaatgtcctttcatctggagattaccgcccgccccgtgctgtggcactggctgctgcgtaccggagggtggaaagtgagcgcagtgaccttggcctatctgatcttttgtctgtgtagcttggtggggattctgcatctccagcgcgcattggtgctacgtaggaaacggaaaaggatgacagatcccacccgccggttc

SEQ ID NO: 156 - protein, 886 aa, unidentified
MDMRVPAQLLGLLLLWLRGARCDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKRGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSAKPTTTPAPRPPTPAPTIASQPLSLRPEAARPAAGGAVHTRGLDFAPRKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMPPPRLLFFLLFLTPMEVRPEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKVSAVTLAYLIFCLCSLVGILHLQRALVLRRKRKRMTDPTRRF

SEQ ID NO: 157 - DNA, 3534 bp, synthetic construct
  source 1..3534: mol_type = other DNA
atggtgtccctgctgcacgccagcaccctggccgtgctgggcgccctgtgcgtgtatggcgccggacacctggaacagccccagatcagcagcaccaagaccctgagcaagaccgccaggctggaatgcgtggtgtccggcatcaccatcagcgccacctccgtgtactggtacagagagagacccggcgaggtcatccagttcctggtgtccatcagctacgacggcaccgtgcggaaagagagcggcatccccagcggcaagttcgaggtggacagaatccccgagaccagcacctccaccctgaccatccacaacgtggagaagcaggacatcgccacctactactgcgccctgtgggagatccaggaactgggcaagaaaatcaaggtgttcggccctggcaccaagctgatcatcaccgacaagcagctggacgccgacgtgagccccaagcctaccatcttcctgcccagcatcgccgagaccaagctgcagaaggccggcacctacctgtgcctgctggaaaagttcttccccgacgtgatcaagatccactgggaggaaaagaagagcaacaccatcctgggcagccaggaaggcaataccatgaaaaccaacgacacctacatgaagttcagctggctgaccgtgcccgagaagagcctggacaaagagcacagatgcatcgtccggcacgagaacaacaagaacggcgtggaccaggaaatcatcttcccccccatcaagaccgatgtgatcacaatggaccccaaggacaactgcagcaaggacgccaacgataccctgctgctgcagctgaccaacaccagcgcctactacatgtatctcctgctgctgctgaagagcgtggtgtacttcgccatcatcacctgctgtctgctgcggcggaccgccttctgctgcaacggcgagaagagcggcagcggcgaaggccgcggcagcctgctgacctgcggcgatgtggaagaaaaccctggcccgatggatatgcgggtaccagcgcaactgcttgggctgcttcttttgtggctgagaggtgctcggtgtgacattcaattgacccagtctccttcttccttgagcgctagcgtcggcgaccgcgtcaccatcacatgcaaagcctcccaggatgtgggaacgtcagtagcctggtaccagcaaaagcctggaaaagcacctaagcttttgatctattggacctctactaggcatacaggggtcccgagtcgcttttccggctccggtagcggcacagatttcaccttcacaatatcttctttacaaccagaagacatcgccacttactactgtcagcagtattctttgtaccgttcctttgggcaaggaacaaaagtagaaataaagagaggtggctctggaagcggcgggtccgggagtggtgggtctggttctgaggtgcagctcgtggagtcaggcggcggggtcgtccagccagggagatctctgaggctgagctgcagcgcgtcggggtttgactttaccacctattggatgagttgggtgcgccaggcccctgggaagggacttgaatggataggcgagatccatcccgacagctccacaatcaattacgcaccttccttgaaagacaggtttacaatctcgagagataacgcaaagaacacactgtttttgcaaatggactcactccgccctgaggacaccggagtgtacttctgcgcgagcctatattttgggttcccctggttcgcatactggggccagggaacccccgtaactgtctcaagcgcaaagccaaccactaccccagcgccgcgaccaccaacaccggcgcccaccatcgcgtcgcagcccctgtctttgcgacctgaggctgctaggcctgccgctggcggagctgtgcacaccagggggttggatttcgcccctcggaagattgaagttatgtatcccccaccctacctcgacaacgaaaaatccaacgggacaataattcacgtaaagggcaaacacctgtgtcccagtccattattccccgggcccagcaaacccttctgggtgctggtggtggtcggtggggtgctggcctgttattccttgctagttaccgtcgccttcattatattctgggtaagatctaagcgttcacgccttctccactctgattacatgaacatgaccccaagaaggcccgggcctaccaggaagcactatcagccgtatgccccacctcgcgacttcgctgcctacaggtctagggtaaaattctctcgcagtgcagagcctcccgcttatcagcaggggcaaaatcaattgtacaatgagctgaacctcggacggcgcgaagagtacgatgtgctggataagcgtcgcgggagagaccccgagatgggaggcaaaccccgcagaaagaatccccaggagggactctataacgagttgcagaaggacaaaatggccgaggcctactcggaaatcggaatgaaaggggaaagaaggcgcggtaaaggccacgacggtctctatcagggcttatctacagctacaaaggatacttacgatgcgctgcacatgcaggcactgccccctcggggatctggcgccaccaatttcagcctgctgaaacaggctggcgacgtggaagagaaccctggacctatggagcggatcagcagcctgatccacctgagcctgttctgggccggagtgatgagcgccatcgagctggtgcccgagcaccagaccgtgcccgtgagcatcggcgtgcccgccaccctgcggtgcagcatgaagggcgaggccatcggcaactactacatcaactggtacagaaagacccagggcaacaccatgaccttcatctaccgggagaaggacatctacggccctggcttcaaggacaacttccagggcgacatcgacatcgccaagaacctggccgtgctgaagatcctggcccccagcgagagggacgagggcagctactactgcgcctgcgacgccctgaagagaaccgacaccgacaagctgatcttcggcaagggcacccgggtgaccgtggagcccagaagccagccccacaccaagcccagcgtgttcgtgatgaagaacggcaccaacgtggcctgcctggtgaaagagttctaccccaaggacatccggatcaacctggtgtccagcaagaagatcaccgagttcgaccccgccatcgtgatcagccccagcggcaagtacaacgccgtgaagctgggcaagtacgaggacagcaacagcgtgacctgcagcgtgcagcacgacaacaagaccgtgcacagcaccgacttcgaggtgaaaaccgactccaccgaccacgtgaagcccaaagagaccgagaacaccaagcagcccagcaagagctgccacaagcccaaggccatcgtgcacaccgagaaggtgaacatgatgagcctgaccgtgctgggcctgcggatgctgttcgccaagacagtggccgtgaacttcctgctgaccgccaagctgttcttcctg

SEQ ID NO: 158 - protein, 1178 aa, synthetic construct
MVSLLHASTLAVLGALCVYGAGHLEQPQISSTKTLSKTARLECVVSGITISATSVYWYRERPGEVIQFLVSISYDGTVRKESGIPSGKFEVDRIPETSTSTLTIHNVEKQDIATYYCALWEIQELGKKIKVFGPGTKLIITDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKSGSGEGRGSLLTCGDVEENPGPMDMRVPAQLLGLLLLWLRGARCDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKRGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSAKPTTTPAPRPPTPAPTIASQPLSLRPEAARPAAGGAVHTRGLDFAPRKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSAEPPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMERISSLIHLSLFWAGVMSAIELVPEHQTVPVSIGVPATLRCSMKGEAIGNYYINWYRKTQGNTMTFIYREKDIYGPGFKDNFQGDIDIAKNLAVLKILAPSERDEGSYYCACDALKRTDTDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 159 - DNA, 1470 bp, synthetic construct
  source 1..1470: mol_type = other DNA
atggcactccccgtcaccgcccttctcttgcccctcgccctgctgctgcatgctgccaggcccgacattgtgctcactcagtcacctcccagcctggccatgagcctgggaaaaagggccaccatctcctgtagagccagtgagtccgtcacaatcttggggagccatcttattcactggtatcagcagaagcccgggcagcctccaacccttcttattcagctcgcgtcaaacgtccagacgggtgtacctgccagattttctggtagcgggtcccgcactgattttacactgaccatagatccagtggaagaagacgatgtggccgtgtattattgtctgcagagcagaacgattcctcgcacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcaccgactacagcataaattgggtgaaacgggcccctggaaagggcctcaaatggatgggttggatcaataccgaaactagggagcctgcttatgcatatgacttccgcgggagattcgccttttcactcgagacatctgcctctactgcttacctccaaataaacaacctcaagtatgaagatacagccacttacttttgcgccctcgactatagttacgcgatggactactggggacagggaacctccgttaccgtcagttccacaacaacccctgcccccagacctcctaccccagcccctacaattgccagccagcctctgagcctgaggcccgaggcttgtagaccagctgctggcggagccgtgcacaccagaggactggatttcgcctgcgacatctacatctgggcccctctggccggcacatgtggcgtgctgctgctgagcctcgtgatcaccctgtactgcaagcggggcagaaagaaactgctgtacatctttaagcagcccttcatgcggcccgtgcagaccacccaggaagaggacggctgctcctgcagattccccgaggaagaagaaggcggctgcgagctgagagtgaagttcagcagatccgccgacgcccctgcctacaagcagggacagaaccagctgtacaacgagctgaacctgggcagacgggaagagtacgacgtgctggacaagcggagaggccgggaccctgaaatgggcggcaagcccagaagaaagaacccccaggaaggcctgtataacgaactgcagaaagacaagatggccgaggcctacagcgagatcggaatgaagggcgagcggagaagaggcaagggccacgatggcctgtaccagggcctgagcaccgccaccaaggacacctatgacgccctgcacatgcaggccctgcctccaaga

SEQ ID NO: 160 - protein, 490 aa, synthetic construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 161 - skipped

SEQ ID NO: 162 - protein, 702 aa, synthetic construct
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 163 - protein, 267 aa, synthetic construct
MALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 164 - protein, 263 aa, synthetic construct
MALPVTALLLPLALLLHAARPDIQMTQTTSSLSASLGDRVTISCRASQDISKYLNWYQQKPDGTVKLLIYHTSRLHSCVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKCLEWLGVIWGSETTYYNSALKSRLTIIKDNSKSQVFLKMNSLQTDDTAIYYCAKHYYYGGSYAMDYWGQGTSVTVSS

SEQ ID NO: 165 - protein, 311 aa, synthetic construct
MDMRVPAQLLGLLLLWLRGARCDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKRGGSGSGGSGSGGSGSEVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSAKPTTTPAPRPPTPAPTIASQPLSLRPEAARPAAGGAVHTRGLDFAPR

SEQ ID NO: 166 - protein, 8 aa, synthetic construct
GITISATS

SEQ ID NO: 167 - protein, 7 aa, synthetic construct
ISYDGTV

SEQ ID NO: 168 - protein, 8 aa, synthetic construct
GEAIGNYY

SEQ ID NO: 169 - protein, 6 aa, synthetic construct
EGSTGY

SEQ ID NO: 170 - protein, 8 aa, synthetic construct
YDSYTSSV

SEQ ID NO: 171 - protein, 6 aa, synthetic construct
NSMFDY

SEQ ID NO: 172 - protein, 7 aa, synthetic construct
ISSIKDK

SEQ ID NO: 173 - DNA, 3411 bp, synthetic construct
  source 1..3411: mol_type = other DNA
atggagtgggccctggccgtgctgctggcctttctgagccctgccagccagaagtccagcaacctggaaggccggaccaagagcgtgatccggcagacaggcagcagcgccgagatcacctgtgacctggccgagggcagcaccggctacatccactggtatctgcaccaagaaggcaaggccccccagcggctgctgtactacgacagctacaccagctccgtggtgctggaaagcggcatcagccccgggaagtacgacacctacggcagcacccggaagaacctgcggatgatcctgcggaacctgatcgagaacgacagcggcgtgtactactgcgccacctgggacggcttctactacaagaagctgttcggcagcggcaccaccctggtggtgacagacaagcagctggacgccgacgtgtcccccaagcctaccatcttcctgccctcaattgctgagacaaagctgcagaaggccggcacctacctgtgcctgctggaaaagttcttcccagacgtgatcaagatccactgggaggaaaagaagtccaacaccatcctgggcagccaagaaggcaacaccatgaagaccaacgacacctacatgaagttcagctggctgaccgtgcccgagaagtccctggacaaagaacaccggtgcatcgtgcggcacgagaacaacaagaacggcgtggaccaagaaatcatcttcccacccatcaagaccgacgtgatcacaatggaccccaaggacaactgcagcaaggacgccaacgataccctgctgctgcagctgacaaacaccagcgcctactacatgtacttgttgctgctgctgaagtccgtggtgtacttcgccatcatcacctgctgtctcctgaggcggaccgccttctgctgcaacggcgagaagagcggatctggcgaaggccgcggcagcctgctgacctgcggcgacgtggaagagaaccctgggcccatggcactccccgtcaccgcccttctcttgcccctcgccctgctgctgcatgctgccaggcccgacattgtgctcactcagtcacctcccagcctggccatgagcctgggaaaaagggccaccatctcctgtagagccagtgagtccgtcacaatcttggggagccatcttattcactggtatcagcagaagcccgggcagcctccaacccttcttattcagctcgcgtcaaacgtccagacgggtgtacctgccagattttctggtagcgggtcccgcactgattttacactgaccatagatccagtggaagaagacgatgtggccgtgtattattgtctgcagagcagaacgattcctcgcacatttggtgggggtactaagctggagattaagggaagcacgtccggctcagggaagccgggctccggcgagggaagcacgaaggggcaaattcagctggtccagagcggacctgagctgaaaaaacccggcgagactgttaagatcagttgtaaagcatctggctataccttcaccgactacagcataaattgggtgaaacgggcccctggaaagggcctcaaatggatgggttggatcaataccgaaactagggagcctgcttatgcatatgacttccgcgggagattcgccttttcactcgagacatctgcctctactgcttacctccaaataaacaacctcaagtatgaagatacagccacttacttttgcgccctcgactatagttacgcgatggactactggggacagggaactagtgttaccgtcagttccacaacaacccctgcccccagacctcctaccccagcccctacaattgccagccagcctctgagcctgagacccgaggcttgtagaccagctgctggcggagccgtgcacaccagaggactggatttcgcctgcgacatctacatctgggcccctctggccggcacatgtggcgtgctgctgctgagcctcgtgatcaccctgtactgcaagcggggcagaaagaaactgctgtacatctttaagcagcccttcatgcggcccgtgcagaccacccaggaagaggacggctgctcctgcagattccccgaggaagaagaaggcggctgcgagctgagagtgaagttcagcagatccgccgacgcccctgcctacaagcagggacagaaccagctgtacaacgagctgaacctgggcagacgggaagagtacgacgtgctggacaagcggagaggccgggaccctgaaatgggcggcaagcccagaagaaagaacccccaggaaggcctgtataacgaactgcagaaagacaagatggccgaggcctacagcgagatcggaatgaagggcgagcggagaagaggcaagggccacgatggcctgtaccagggcctgagcaccgccaccaaggacacctatgacgccctgcacatgcaggccctgcctccaagaggcagcggcgccaccaatttcagcctgctgaaacaggctggagacgtggaagaaaaccctggcccgatggccatgctgctgggagccagcgtgctgatcctgtggctgcagcccgattgggtcaacagccagcagaagaacgacgaccagcaagtgaagcagaacagccccagcctgagcgtgcaggaaggccggatcagcatcctgaactgcgactacaccaactctatgttcgactacttcctgtggtacaagaagtaccccgccgagggccccaccttcctgatctccatcagcagcatcaaggacaagaacgaggacggccggttcaccgtgtttctgaacaagagcgccaagcacctgagcctgcacatcgtgcctagccagcctggcgatagcgccgtgtacttttgtgccgccagcagccccatcagaggctacaccggcagcgacaagctgatcttcggcaagggcaccagagtgaccgtggaacccagaagccagccccacaccaagcccagcgtgttcgtgatgaagaacggcaccaacgtggcctgcctcgtgaaagagttctaccccaaggacatccggatcaacctggtgtccagcaagaagatcaccgagttcgaccccgccatcgtgatcagcccctccggcaagtacaacgccgtgaagctggggaagtacgaggacagcaacagcgtgacctgctccgtgcagcacgacaacaagaccgtgcacagcaccgatttcgaagtgaaaaccgactccaccgaccacgtgaagcccaaagagacagagaacaccaagcagcccagcaagagctgccacaagcccaaggccatcgtgcacaccgagaaagtgaacatgatgagcctgaccgtgctgggcctgcggatgctgttcgccaaaaccgtggccgtgaacttcctgctgaccgccaagctgttctttctg

SEQ ID NO: 174 - protein, 1137 aa, synthetic construct
MEWALAVLLAFLSPASQKSSNLEGRTKSVIRQTGSSAEITCDLAEGSTGYIHWYLHQEGKAPQRLLYYDSYTSSVVLESGISPGKYDTYGSTRKNLRMILRNLIENDSGVYYCATWDGFYYKKLFGSGTTLVVTDKQLDADVSPKPTIFLPSIAETKLQKAGTYLCLLEKFFPDVIKIHWEEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKSGSGEGRGSLLTCGDVEENPGPMALPVTALLLPLALLLHAARPDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASSPIRGYTGSDKLIFGKGTRVTVEPRSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL